SEQUENCE LISTING (4 sequences)

SEQ ID NO: 1 - DNA, 21 bp, Artificial Sequence
  Synthetic- FoxM1 primer
cacttggattgaggaccactt

SEQ ID NO: 2 - DNA, 20 bp, Artificial Sequence
  Synthetic- FoxM1 primer
gtcgtttctgctgtgattcc

SEQ ID NO: 3 - DNA, 20 bp, Artificial Sequence
  Synthetic- mouse cyclophilin primer
cttgtccatggcaaatgctg

SEQ ID NO: 4 - DNA, 21 bp, Artificial Sequence
  Synthetic- mouse cyclophilin primer
tgatcttcttgctggtcttgc

(19) United States
(12) Patent Application Publication
(10) Pub. No.: US 2023/0248706 A1
(43) Pub. Date: Aug. 10, 2023

(54) METHODS AND COMPOSITIONS FOR THE TREATMENT OF COVID-19 AND ASSOCIATED RESPIRATORY DISTRESS AND MULTI-ORGAN FAILURE, SEPSIS, ACUTE RESPIRATORY DISTRESS SYNDROME, AND CARDIOVASCULAR DISEASES

(71) Applicant: Ann And Robert H. Lurie Children's Hospital of Chicago, Chicago, IL (US)

(72) Inventor: Youyang Zhao, Deerfield, IL (US)

(21) Appl. No.: 18/002,940

(22) PCT Filed: Jun. 24, 2021
(86) PCT No.: PCT/US2021/070767

§ 371 Date: Dec. 22, 2022

Related U.S. Application Data
(60) Provisional application No. 63/044,356, filed on Jun. 26, 2020.

(51) Int. Cl. A61K 31/4439; A61K 31/655; A61K 31/472; A61K 31/4418; A61K 9/51; A61P 11/00

(52) U.S. Cl. CPC A61K 31/4439; A61K 31/655; A61K 31/472; A61K 31/4418; A61K 9/5153; A61P 11/00

(57) ABSTRACT

Disclosed herein are methods and compositions for treatment of COVID-19 and associated sepsis, respiratory distress and organ failure, acute respiratory distress syndrome, sepsis, infection-induced organ failure, restenosis, critical limb ischemia, and vascular diseases associated with impaired endothelial regeneration, vascular repair, and vascular regeneration. In some embodiments, the methods include administering an effective amount of one or more of a) Dexamethasone, Resveratrol, N-acetylcysteine, Apocynin, Ebselen, APX-115, NOX2 inhibiting peptide, NOX2 inhibiting nucleic acid, Thienopyridine, orb) Selisistat, AG-1031, rabeprazole, phenazopyridine, roxadustat, molidustat, vadadustat, desidustat, decitabine, azacytidine, and analogues thereof, FOXM1 expressing nucleic acid, HIF1A expressing nucleic acid, SIRT1 inhibiting nucleic acid, EGLN1 inhibiting nucleic acid, or c) combination of one of a with one of b to a subject in need thereof. In some embodiments, the monotherapy or combination therapy is particularly useful for treatment of an elderly subject, and is useful to treat a subject at any age.

### CROSS-REFERENCE TO RELATED APPLICATIONS

This application claims the benefit of U.S. Provisional Application No. 63/044,356, filed Jun. 26, 2020, the entire content of which is incorporated herein by reference in its entirety.

### STATEMENT REGARDING FEDERALLY SPONSORED RESEARCH OR DEVELOPMENT

This invention was made with government support under HL123957, HL125350, HL133951, HL140409, and HL077806, awarded by the National Institutes of Health. The government has certain rights in the invention.

### BACKGROUND

Acute respiratory distress syndrome (ARDS) is a form of acute-onset hypoxemic respiratory failure with bilateral pulmonary infiltrates, which is caused by acute inflammatory edema of the lungs not attributable to left ventricular heart failure. The most common underlying causes of ARDS include sepsis, severe pneumonia, inhalation of harmful substance, burn, as well as major trauma with shock. Endothelial injury characterized by persistently increased lung microvascular permeability resulting in protein-rich lung edema is a hallmark of ARDS. Despite recent advances in the understanding of the pathogenesis, there are currently no effective pharmacological, cell, or gene-based treatment of the disease, and the mortality rate is as high as 40%. Compared to young adult patients, the incidence of acute lung injury (ALI)/ARDS resulting from sepsis, pneumonia, and flu in elderly patients (≥60 yr) is as much as 20-fold greater and the mortality rate is 10-20-fold greater. However, the underlying causes are poorly understood. In addition, crucially little is known about how aging influences mechanisms of endothelial injury, regeneration and vascular repair as well as resolution of inflammatory injury.

COVID-19 caused by SARS-CoV2 infection is considered as a systemic disease that primarily injures the vascular endothelium although the portal for the virus is inhalational. Clinically, soon after onset of respiratory distress from COVID-19, patients develop severe hypoxiemia, and interstitial rather than alveolar edema. Pathological examinations reveal that the lungs have extensive hemorrhages and are expanded with exudatives with high incidence of thrombi in small vessels, pointing to excessive vascular endothelium injury. In addition to respiratory distress, cardiovascular complication with widespread macro- and micro-thromboses is another feature of severe COVID-19. The morbidity and mortality of COVID-19 patients in elderly patients are much higher than that in young adult patients. In New York city, the death rates of COVID-19 patients are 168, 1,540, 5,020, and 12,630 per million people in age group of 18-44, 45-64, 65-74, and ≥75 years old, respectively. In Italy, the mortality rate is less than 0.3%, for 20-39 years old COVID-19 patients, 10.1% for 60-69 years old while more than 25% for ≥70 year old COVID-19 patients. It is unknown why the severity and mortality are so much higher in elderly patients, and there is no effective treatment. The current therapy is largely supportive. Besides virus eradication, novel therapeutics to inhibit injury and promote repair and recovery is also very important.

### SUMMARY

Disclosed herein are methods and compositions for treatment of COVID-19, and COVID-19-related conditions such as COVID-19-related sepsis, COVID-19-related respiratory distress, and multi-organ failure. In addition, methods and compositions are disclosed herein to treat sepsis, acute respiratory distress syndrome (ARDS), acute inflammatory injury, infection-induced organ failure characterized by vascular injury and also to treat critical limb ischemia, and restenosis, and vascular diseases associated with impaired endothelial regeneration, vascular repair, and vascular regeneration in a subject in need thereof.

In some embodiments, the methods include administering to the subject an effective amount of one or more compounds that inhibit endothelial injury and inflammation. Exemplary compounds include, but are not limited to N-acetyl cysteine (NAC), NOX2 inhibitors (Thienopyridine, NOX2ds-tat), pan-NOX inhibitors (Apocynin, Ebselen, APX-115), Reseveratrol (trans-E-resveratrol, “RV”) nanoparticles and analogues thereof (e.g., RV-loaded nanoparticles comprising poly(D,L-lactic-co-glycolic acid) (PLGA)-b-long linker poly(ethylene glycol) (PEG, e.g. 5,000 Da) copolymer, and RV-loaded nanoparticles comprising poly(D,L-lactic acid) (PLA)-b-PEG copolymer), and NOX2 inhibiting nucleic acid.

In some embodiments, the methods include administering to the subject an effective amount of one or more compounds that promote endothelial regeneration and vascular repair. Exemplary compounds include, but are not limited to Decitabine (e.g. Dacogen, INQOVI) and its analogues (e.g., Vidaza, ONUREG), prolyl hydroxylase (PHD) inhibitors (e.g., roxadustat (FG-4592), molidustat, vadadustat, and desidustat, and dimethyoxalylglycine (DMOG) analogs), Sirtuinl (SIRT1) inhibitors (e.g., Selisistat, AG1031) and its analogues, rabeprazol (e.g., Aciphex) and its analogues, phenazopyridine (e.g., Pyridium) and its analogues; SIRT1 inhibiting nucleic acid, EGLN1 inhibiting nucleic acid, HIF1A expressing nucleic acid, FOXM1 expressing nucleic acid.

In some embodiments, the methods include administering to the subject a combination therapy comprising (a) an effective amount of one or more compounds that inhibits endothelial injury and inflammation, and (b) an effective amount of one or more compounds that promote endothelial regeneration and vascular repair.

By way of example, but not by way of limitation, in some embodiments the combination therapy includes but is not limited to (a) one or more of the inhibitors of inflammatory injury including Dexamethasone, NAC, Apocynin, Ebselen, APX-115, Thienopyridine, or NOX2ds-tat, RV nanoparticles, NOX2 inhibiting nucleic acid, and (b) one or more of the vascular reparative drugs including Decitabine (e.g., Dacogen, INQOVI, Vidaza, ONUREG), Selisistat, AG-1031, rabeprazol, phenazopyridine or DMOG analogues roxadustat, molidustat, vadadustat, and desidustat, SIRT1 inhibiting nucleic acid, EGLN1 inhibiting nucleic acid, HIF1A expressing nucleic acid, FOXM1 expressing nucleic acid.

The disclosed methods may include administering to a subject in need thereof an effective amount of one or more nucleic acid-based therapeutic agents for treating one or more of ARDS, sepsis, COVID-19, and COVID-19 respiratory distress and multi-organ failure. The inhibiting nucleic acid-based therapeutic may be, but is not limited to an antisense oligonucleotide, a small interfering RNA (siRNA), shRNA, and a guide RNA-based genome editing system.

The disclosed methods may be performed on any suitable subject. In some embodiments, the subject is a human and the subject is elderly, e.g., 60 years old or older.

Also disclosed herein are methods and compositions for treating one or more of: cardiovascular diseases including restenosis (to prevent or treat restenosis after percutaneous coronary intervention), and peripheral vascular disease, e.g., critical limb ischemia (to promote angiogenesis) in a subject in need thereof, the method comprising administering to the subject an effective amount of one or more of (a) a HIF1A expressing nucleic acid, (b) a FOXM1 expressing nucleic acid, (c) a SIRT1 inhibiting nucleic acid, (d) an EGLN1 inhibiting nucleic acid, (e) rabeprazol or analogues thereof, (f) Phenazopyridine or analogues thereof, (g) dimethyoxalylglycine (DMOG) analogues thereof (e.g., roxadustat, molidustat, vadadustat, and desidustat), (h) Selisistat or AG-1031 or analogues thereof.

### BRIEF DESCRIPTION OF THE FIGURES

FIG. 1. Genetic lineage tracing demonstrating lung resident ECs are the cells of origin for endothelial regeneration following polymicrobial sepsis-induced injury. (A) Schematic illustration of the lineage-tracing strategy. Tam=Tamoxifen. (B) Flow cytometry analysis of GFP⁺ cells and ECs (CD45⁻CD31⁺) in mouse lungs. (C) Quantification of GFP⁺ ECs in EndoSCL-Cre^{ERT2}/mTmG mouse lungs demonstrating 95% of labeling efficiency. At 1 mo. post-tamoxifen or vehicle treatment, lung tissues were collected for cell isolation and were then immunostained with anti-CD45 and anti-CD31 antibodies. CD45⁻ cells were gated for CD31⁺ and GFP⁺ analysis. (D) Representative confocal images of lungs of young adult mice (3-5 mos. old) showing changes in GFP-labeled ECs. At 48h post-CLP, loss of GFP⁺ ECs was evident in pulmonary vessel (arrows) and alveolar capillaries (arrowheads). Red, tdTomato+ (non-ECs); Green, GFP⁺ cells (ECs). Blue, DAPI. At 144h post-CLP, vascular integrity was fully recovered as evident by intact green lining as seen in Sham control lungs. Br, bronchiole; V, vessel. Scale bar, 20 μm. (E, F) FACS analysis demonstrating loss of GFP⁺ ECs at 48h and steady recovery of GFP⁺ ECs during the repair phase in young adult mice which was returned to the level seen in sham-operated mice at 144h post-CLP. Lung cells were CD45-gated, and GFP⁺ population was quantified. (G) FACS analysis showing impaired recovery of lung GFP⁺ cells following CLP challenge in aged mice (19-21 mos. old). Bars represent means. ** P<0.01 versus Sham; **** P<0.0001 versus Sham. One-way ANOVA with Dunnett's post-hoc multiple comparison test. (H) FACS analysis of changes of CD45⁺/GFP⁺ cells demonstrating CD45⁺/GFP⁺ cells were not involved in endothelial regeneration following CLP. At various times following CLP challenge, lungs from tamoxifen-treated EndoSCL-CreERT2/mTmG mice were collected for cell isolation followed by immunolabeling with anti-CD45 antibody. Sham, 144h post-Sham. (I, J) Bone marrow transplantation study demonstrating little contribution of bone marrow-derived cells in lung endothelial regeneration. Bone marrow cells isolated from mTmG/EndoSCL-Cre^{ERT2} mice were transplanted to lethally irradiated C57BL/6 WT mice to generate chimeric mice. Upon tamoxifen treatment, bone marrow-derived ECs were labeled with GFP in these chimeric mice. FACS analysis shows that the percentage of CD45⁻GFP⁺ cells (e.g. ECs) in lungs of the chimeric mice at 4 weeks post-tamoxifen treatment was similar to mice at 144h post-CLP challenge. Similarly, CD45⁺GFP⁺ population was not changed (J).

FIG. 2. Defective endothelial proliferation and vascular repair in aged lungs following polymicrobial sepsis. (A) Representative micrographs of BrdU immunostaining showing defective EC proliferation in aged lungs. Cryosections of lungs (5 μm) collected at 96h post-CLP were immunostained with anti-BrdU antibody to identify proliferating cells (green) and with anti-CD31 and vWF antibodies to identify ECs (red). Nuclei were counterstained with DAPI (blue). Arrows point to proliferating ECs. Aged, 20 mos. old; young, 3 mos. old; Scale bar, 50 μm. (B) Quantification of cell proliferation in mouse lungs. Three consecutive cryosections from each mouse lung were examined, the average number of BrdU⁺ nuclei was used. (C) Lung vascular permeability assessed by EBA extravasation assay. Following perfusion to be free of blood, lung tissues were collected at indicated times post-CLP for EBA assay. (D) Lung wet/dry weight ratio. At 96h post-CLP, lung tissues were collected and dried at 60° C. for 3 days for calculation of wet/dry ratio. *** P<0.001; **** P<0.0001. Student's t test.

FIG. 3. Impaired resolution of lung inflammation in aged mice following CLP challenge. (A) Representative micrographs of H & E staining of lung sections. At 96h post-CLP, lungs were fixed for sectioning and H & E staining. Arrows indicate perivascular leukocyte sequestration. Scale bar: 120 μm. (B) MPO activities in lung tissues. Lung tissues at indicated times post-CLP challenge were collected for MPO activity determination. MPO activity was calculated as OD460/min/g lung tissue. (C, D) Quantitative RT-PCR analysis showing marked increase of expression of pro-inflammatory genes in lungs of aged mice at 96h post-CLP compared to young mice. ** P<0.01; *** P<0.001. Student's t test.

FIG. 4. Aging impairs resolution of inflammatory lung injury following LPS challenge. (A) Persistent increase of lung vascular permeability in aged mice following LPS challenge. Lungs of aged (19-21 mos. old) and young adult (3-5 mos. old) mice were collected at various times for EBA flux assay. (B) Lung edema in aged mice at 72h post-LPS. (C) Sustained increase of MPO activity in aged lungs at indicated times following LPS challenge. (D) Representative micrographs of H & E staining showing perivascular neutrophil accumulation in aged lungs at 72h post-LPS. Arrows point to neutrophil accumulation. Br, bronchiole, V, vessel. Scale bar, 60 μm. (E) Quantitative RT-PCR analysis demonstrating markedly elevated expression of proinflammatory genes in aged lungs at 72h post-LPS. (F) EBA flux assay demonstrating aging impaired vascular repair. WT mice at indicated ages were challenged with LPS (mice at age of 3-9 mo. were challenged with 2.5 mg/kg, and at age of 12-21 mo. with 1.25 mg/kg LPS). At 72h post-LPS, lungs were collected for EBA extravasation assay. (G) Lung MPO activity in mice at various ages at 72h post-LPS challenge. * P<0.05; *** P<0.001; **** P<0.0001. Student's t test.

FIG. 5. Defective endothelial proliferation and failure of FoxM1 induction in aged lungs following LPS challenge. (A) Representative micrographs of immunostaining showing inhibited endothelial proliferation in aged lungs at 72h post-LPS challenge. Lung cryosections were immunostained with anti-BrdU (green), anti-CD31/and anti-vWF (red, ECs). Nuclei were counterstained with DAPI (blue). Arrows point to proliferating ECs. Scale bar, 50 (B) Quantification of cell proliferation in mouse lungs at basal and 72h post-LPS challenge. (C) Quantitative RT-PCR analysis of FoxM1 expression in mouse lungs at indicated times post-LPS. (D) Quantitative RT-PCR analysis showing marked increase of expression of FoxM1-target genes at 72h post-LPS in lungs of young mice but not aged mice. ** P<0.01; *** P<0.001; **** P<0.0001. Student's t test.

FIG. 6. Transgenic expression of FoxM1 normalized resolution of inflammatory lung injury and promoted survival of aged mice. (A) EBA flux assay showing normalized vascular repair in aged FOXM1^{Tg} (Tg) mice following LPS challenge. WT and FOXM1^{Tg} (Tg) mice at age of 19-21 mo. were challenged with LPS (1 mg/kg, i.p.). Lung tissues were collected at the indicated times for EBA extravasation assay. (B) Lung MPO activity assessment demonstrating normal resolution of inflammation in aged FOXM1^{Tg} mice. (C) Quantitative RT-PCR analysis showing marked increase of expression of pro-inflammatory genes in lungs of aged WT but FOXM1^{Tg} mice at 72h post-LPS. (D) Aging impaired survival of WT mice whereas transgenic expression of FoxM1 promoted survival of aged mice. WT mice at age of 3-5 mo. old (Young), and age of 19-21 mo. old (Aged WT) and FOXM1^{Tg} mice at age of 19-21 mo. old (Aged Tg) were challenged with a relatively high dose of LPS (1.5 mg/kg, i.p.). Survival rate was then recorded in 7 days. * P<0.05; ** P<0.01; *** P<0.001; **** P<0.0001. Student's t test (A-C). * P<0.05 versus Aged Tg, **** P<0.0001 versus Aged WT. Log-rank (Mantel-Cox) test (D).

FIG. 7. Therapeutic expression of FoxM1 in lung ECs of aged WT mice reactivated lung endothelial proliferation and vascular repair and normalizes inflammation resolution following LPS challenge. (A) Representative Western blotting demonstrating marked increase of FoxM1 expression in lungs of aged mice transduced with FOXM1 (FOX) plasmid DNA. Mixture of liposome: FOXM1 plasmid DNA expressing human FOXM1 under the control of human CDH5 promoter were administered retro-orbitally to aged WT mice (19-21 mo. old) at 12h post-LPS (1 mg/kg, i.p.). Each mouse received 50 μg plasmid DNA in a bolus injection. Lungs were collected at 72h post-LPS for Western blotting. (B) Marked decrease of lung vascular permeability in FOXM1-transduced mice at 72h post-LPS challenge. (C, D) normal resolution of lung inflammation in FOXM1-transduced mice at 72h post-LPS in contrast to vector DNA-transduced mice evident by diminished MPO activity (C) and expression of proinflammatory genes (D). (E, F) Forced expression of FoxM1 in lung ECs of aged mice reactivated lung EC proliferation. At 72h post-LPS challenge, lung tissues were collected for cryosectioning and immunostaining with anti-BrdU (green), and anti-CD31 and anti-vWF (markers for ECs, red). Nuclei were counterstained with DAPI (E). Arrows point to proliferating ECs. Scale bar, 60 μm. The BrdU⁺ ECs and non-ECs were quantified (F). (G) Quantitative RT-PCR analysis showing marked induction of cell cycle genes (FOXM1 target genes) in lungs of FOXM1-transduced mice at 72h post-LPS but not in lungs of vector DNA-transduced mice. (H, I) Nanoparticle delivery of FOXM1 plasmid DNA in mice at age of 25 mo. old activated vascular repair and inflammation resolution. Mixture of nanoparticle: FOXM1 plasmid DNA or vector DNA was administered retro-orbitally to elderly mice at age of 25 mo. old at 24h post-LPS (0.25 mg/kg, i.p.). Each mouse received 15 μg DNA in a bolus injection. At 96h post-LPS, lung tissues were collected for EBA extravasation assay (H) and MPO activity determination (I). ** P<0.01; *** P<0.001; **** P<0.0001. Student's t test.

FIG. 8. Failure of FOXM1 induction in pulmonary vascular ECs of elderly COVID-19 patients in contrast to middle-aged patients. (A) Representative micrographs of RNAscope in situ hybridization staining of human lung sections showing marked induction of FOXM1 expression in pulmonary vascular ECs of middle-aged COVID-19 patients but not in elderly patients. Lung autopsy tissues were collected from COVID-19 patients and healthy donors (normal) for paraffin-sectioning and immunostaining. Anti-CD31 antibody was used to immunostain ECs (green). FOXM1 mRNA expression (purple) was detected by RNAscope in situ hybridization. Nuclei were counterstained with DAPI. Arrow point to FOXM1 expressing ECs. V, vessel. Scale bar, 50 μm. (B) Quantification of endothelial expression of FOXM1. FOXM1 was markedly induced in ECs of middle-aged COVID-19 patients (50, 56, 56) but not in elderly COVID-19 patients (82, 84, 84). FOXM1 expression was quantified in 14-33 vessels of each subject. Bars (red) represent means. ** P<0.01, Kruskal-Wallis test (non-parametric).

FIG. 9. Identification of Rabeprazole and Phenazopyridine as HIF activators which activate FoxM1-dependent endothelial regeneration and vascular repair program in aged lungs. (A-C) Rabeprazole activation of FoxM1-dependent endothelial regeneration and vascular repair program in aged lungs leading to resolution of inflammatory injury. Twenty-two mo. old mice were challenged with LPS (1.5 mg/kg, i.p., LPS from E. coli 055:B55 was purchased from Santa Cruz). At 6h post-LPS, the mice were treated with Rabeprazole and again at 24h post-LPS. At 72h post-LPS, lung tissues were collected for EBA flux assay to measurement vascular permeability (A), MPO activity to determine neutrophil sequestration (B) and quantitative RT-PCR analysis to quantify FoxM1 expression (C). (D) Phenazopyridine treatment promoted pulmonary vascular recovery in aged mice after sepsis challenge. Twenty-two mo. old mice were challenged with LPS (1.5 mg/kg, i.p.). At 6h post-LPS, the mice were treated with Phenazopyridine and again at 24h post-LPS. At 72h post-LPS, lung tissues were collected for EBA flux assay to measurement vascular permeability.

FIG. 10. Rabeprazol activation of FoxM1-dependent endothelial regeneration and vascular repair program in lungs of young adult mice. (A, B) Rabe treatment promoted vascular repair and induced FoxM1 expression. 3-5 mo. old mice were challenged with LPS (2.5 mg/kg, i.p.). At 6h post-LPS, the mice were treated with Rabeprazol (Rabe, 20 mg/kg, oral) and again at 24h post-LPS or PBS vehicle (Veh). At various times post-LPS, lung tissues were collected for EBA flux assay to measurement vascular permeability (A), and quantitative RT-PCR analysis to quantify FoxM1 expression at 56h post-LPS (B). (C) WT or Hif1a EC-specific knockout mice (Hif1a KO) were challenged with LPS and then treated with Rabe or vehicle. Lung tissues were collected at 52h post-LPS for EBA assay. (D) WT or Foxm1 EC-specific knockout mice (Foxm1 KO) were challenged with LPS and then treated with Rabe or vehicle. Lung tissues were collected at 52h post-LPS for EBA assay.

FIG. 11. Genetic deletion of Egln1 in ECs promotes normal vascular repair and resolution of inflammatory lung injury in aged mice following sepsis challenge. 21 months old WT or Egln1^{ΔEC} mice were challenged with LPS and lung tissues were collected at various times for vascular permeability (EBA Flux) (A) and lung inflammation (MPO activity) (B) assessments as well as QRT-PCR analysis of FoxM1 expression (C). **, P<0.01.

FIG. 12. DMOG activation of FoxM1-dependent endothelial regeneration and vascular repair program in aged lungs leading to resolution of inflammatory injury. 21 mo. old mice were challenged with LPS. At 12h or 24h post-LPS, the mice were treated with DMOG (8 mg/mouse, i.p.). At 72h post-LPS, lung tissues were collected for EBA flux assay to measurement vascular permeability (A), MPO activity to determine neutrophil sequestration (B) and quantitative RT-PCR analysis to quantify expression of FoxM1 (C) and pro-inflammatory cytokines TNF-α (D) and IL-6 (E). *, P<0.05.

FIG. 13. FG-4592 (i.e. roxadustat) treatment activated vascular repair and induced FoxM1 expression but also marked increase of pro-inflammatory cytokine expression. 21 mo. old mice were challenged with LPS (0.5 mg/kg, i.p.). At 24h post-LPS, the mice were treated with FG-4592 (25 mg/kg, oral). At 72h post-LPS, lung tissues were collected for EBA flux assay (A), MPO activity measurement (B) and quantitative RT-PCR analysis to quantify expression of FoxM1 (C).

FIG. 14. Endothelial SIRT1 deficiency promotes normal vascular repair and resolution of inflammation in aged mice. (A) QRT-PCR analysis demonstrating a marked increase of SIRT1 expression in aged WT mice after LPS challenge. Lung tissues were collected at basal and 96h post-LPS challenge from aged WT and EC-specific Sirt1 knockout mice (Sirt1^{ΔEC}). (B, C) At indicated times post-LPS, lung tissues were collected for vascular permeability (EBA flux) (B) and inflammation (MPO activity) assessment (C). Age, 20-24 months, LPS 1-1.75 mg/kg. *P<0.05; ** P<0.01; ***P<0.001.

FIG. 15. SIRT1 inhibition by EX-527 (i.e. Selisistat) treatment reactivates FoxM1 expression and normalizes vascular repair in lungs of aged mice. Nineteen months old WT mice were challenged with LPS. A group of mice were treated with the SIRT1 inhibitor EX-527 (also known as Selisistat) (7 mg/kg, i.p.) at 1 h prior to LPS challenge. Lung tissues were collected at 72h for assessment of vascular permeability by EBA flux assay (A) and lung inflammation indicative of MPO activity (B), and QRT-PCR analysis of FoxM1 expression (C). **P<0.01; ***P<0.001.

FIG. 16. Aged mice exhibited much more severe lung injury than young adult mice following endotoxemia. WT Mice at age of 4 mo. (Young) or 20 mo. (Aged) were challenged with the same dose of LPS. At 24h post-LPS, lung tissues were collected for determination of vascular permeability (EBA flux) (A) and lung inflammation (MPO activity) (B). n=3 mice/Basal group at basal and 4 mice/LPS group. **P<0.01.

FIG. 17. Upregulated expression of NOX2 but not NOX4 in lungs of aged mice. Lung tissues were collected from young adult (4 months old) and aged (20 months old) mice at basal and different times following LPS challenge for assessment of NOX2 and NOX4 expression by QRT-PCR analysis. * P<0.05; ** P<0.01.

FIG. 18. Marked inhibition of inflammatory lung injury of aged mice with EC-specific disruption of NOX2 whereas high mortality of NOX4-deficient mice. (A, B) NOX2 was markedly induced in NOX4-deficient ECs of aged mice. Twenty months old mice were administered i.v. with mixture of PLGA-PEG/PEI nanoparticle: Plasmid DNA expressing Cas9 under the control of CDH5 promoter and NOX2 or NOX4-specific guide RNA, scrambled (Scr) NOX2 RNA, or both NOX2/4 guide RNA to knockdown NOX2 or NOX4 in lung ECs, respectively. NOX2/4=knockdown of both NOX2 and NOX4. Seven days later, lung tissues were collected for Western blotting (A), and EC and non-EC isolation which were used for QRT-PCR analysis of NOX2 expression (B). ***P<0.001. (C-G) EC-specific knockdown of NOX2 resulted in inhibited lung vascular injury in aged mice. At 7 days post-nanoparticle administration, aged mice were challenged with LPS. Lung tissues were collected at 24h post-LPS for assessment of vascular permeability (EBA Flux) (C), and lung inflammation by MPO activity (D) and QRT-PCR analysis of proinflammatory cytokine expression (E-G). All 5 aged mice with NOX4 knockdown died within 24h. **P<0.01 versus scramble (Scr) controls (i.e. WT).

FIG. 19. Marked reduction of cell apoptosis in NOX2- or NOX2/4-deficient lungs of aged mice following LPS challenge. (A) Representative micrographs of immunofluorescent staining demonstrating reduced cell apoptosis in aged mice with NOX2 deficiency in ECs at 24h post-LPS challenge. (B) Quantification of cell apoptosis. **P<0.001 versus either NOX2 or NOX2/4-deficient mice.

FIG. 20. Marked induction of NOX2 but not NOX4 in senescent human lung ECs and N-acetyl cysteine (NAC) inhibition of apoptosis in aged ECs. (A) Representative micrographs of β-galactosidase staining (blue) demonstrating senescence of human lung microvascular ECs (HLMVECs) at passage 16 in culture in contrast to passage 6. (B) Western blotting demonstrating marked induction of NOX2 but not NOX4 in senescent ECs. p16^{INK4a} is a marker for cell senescence. (C) Representative micrographs of DCF staining demonstrating excessive production of ROS in passage 16 (P16) HLMVECs by TNFα/CHX (Cycloheximide) treatment, which was markedly reduced by NAC treatment. (D) Quantification of ROS production. (E) Representative micrographs of TUNEL staining (green) of human lung ECs. (F) Quantification of TUNEL-positive nuclei demonstrating that treatment with TNF-α+CHX induced a marked increase of apoptosis in senescent ECs (passage 16) but NAC treatment markedly inhibited TNF-α+CHX-induced apoptosis. ***P<0.001.

FIG. 21. NAC treatment of aged mice reduces inflammatory lung injury. Young (4 months old) and aged (21.5 months old) mice were challenged with the same dose of LPS (2 mg/kg, i.p.). 2h later, aged mice were treated with NAC (120 mg/kg, oral) and lung tissues were collected at 24h post-LPS for EBA (A) and MPO (B) assays and quantitative RT-PCR analysis of proinflammatory gene 116 expression (C).

FIG. 22. Unique formulation of the Resveratrol-loaded PLGA-PEG nanoparticles highly efficiently inhibited lung injury induced by polymicrobial sepsis. (A) A diagram showing the generation of Resveratrol (RV)-loaded PLGA-PEG nanoparticles with different PEG size. PLGA-b-PEG=poly(D,L-lactic-co-glycolic acid)-b-PEG=poly(ethylene glycol) copolymer, PEGs=MW600 Da, PEGl=2000 Da. (B, C) RV-PLGA-PEG2000 nanoparticle was markedly efficient in inhibiting inflammatory lung injury induced by polymicrobial sepsis. Mice were subjected to cecal ligation and puncture (CLP) surgery to induce polymicrobial sepsis. 3h post-CLP, the mice were randomized to receive RV-PLGA, RV-PLGA-PEGs or RV-PLGA-PEGl nanoparticles, respectively. At 36h post-CLP, lung tissues were collected for assessment of vascular permeability (EBA Flux) (B) and inflammation (MPO activity) (C). (D) RV-PLGA-PEG2000 nanoparticle treatment promoted mice survival from polymicrobial sepsis. ***P<0.001.

FIG. 23. Decitabine treatment had no effect on inflammatory lung injury in young adult mice following LPS challenge. Adult mice (3-5 mo. old) were challenged with LPS (2.5 mg/kg, i.p.) and then treated with PBS or Decitabine (Decit) at various doses (mg/kg, i.p.) at 4h post-LPS challenge. Lung tissues were collected at 24h post-LPS for measurement of MPO activity (A) and EBA flux (B).

FIG. 24. Decitabine treatment promoted vascular repair and resolution of inflammation in aged mice but not in young adult mice. Aged WT mice (21 mos. old) or young adult (3 mos. old) mice were challenged with LPS (i.p.) and then treated with either Decitabine (LPS+D) or vehicle (PBS) (LPS) at 24 and 48h post-LPS. Decitabine (0.2 mg/kg, i.p.) was administered once a day. Lung tissues were collected at 96h post-LPS for EBA (A, C) and MPO (B, D) assays. **P<0.01; ****P<0.0001. Student's t test. ns, not significant.

FIG. 25. Decitabine treatment induces FoxM1 expression and promotes FoxM1-dependent survival of aged mice following LPS challenge. (A) Decitabine treatment induced endothelial cell proliferation, i.e. endothelial regeneration in aged lungs. ECs of aged lungs were immunostained with anti-vWF (red) and nuclei were counted with DAPI (blue). Arrows point to BrdU+ ECs. Scale bar, 20 μm. (C, D) Decitabine treatment induced FoxM1 expression in aged lungs. Aged WT mice (21 mos. old) were challenged with LPS (i.p.) and then treated with either Decitabine (LPS+D) or vehicle (PBS) (LPS) at 24 and 48h post-LPS. Lung tissues were collected at 96h post-LPS for RNA isolation and quantitative RT-PCR analysis of FoxM1 (B) and FoxM1 target genes essential for cell cycle progression (C). (D) Decitabine treatment promoted survival of aged mice in a FoxM1-dependent manner. Aged (20-22 mos. old) WT or Foxm1 EC-specific knockout mice (CKO) were challenged with LPS (1.5 mg/kg, i.p.) and then treated with Decitabine (LPS+D) at 24 and 48h post-LPS. Mortality rate were monitored for 96h. *P<0.05; **P<0.01; ****P<0.0001. Student's t test (A-C). Log-rank (Mantel-Cox) test (D).

### DETAILED DESCRIPTION

Described herein are methods and compositions useful for inhibition of vascular injury and inflammation, and promotion of endothelial cell regeneration, vascular repair, and resolution of inflammatory injury as well as inhibiting anemia and promoting angiogenesis. In some embodiments, the methods and compositions disclosed herein are particularly useful in the aged subjects.

It has been shown that the incidence of acute respiratory distress syndrome (ARDS) resulting from sepsis is as much as 20-fold greater in elderly patients (e.g., someone who is 60 years of age or older) than in young adult patients, and the mortality rate of elderly COVID-19 patients is also 10-80-fold greater. Persistent endothelial injury leading to tissue edema and severe hypoxiemia is a hallmark of these conditions. The underlying causes are poorly understood and current therapy is merely supportive. In contrast, the methods and compositions disclosed herein show that treatment with N-acetyl cysteine (NAC), or NOX2 inhibiting nucleic acid markedly inhibit sepsis-induced lung inflammation and vascular injury and promote survival in aged mice. Additionally or alternatively, in some embodiments, nanoparticle-based gene therapy with FoxM1, or treatment with rabeprazole, phenazopyridine, Decitabine, DMOG or its analogue FG-4592 (roxadustat), or EX-527 (Selisistat) alone, or genetic deletion of SIRT1 or EGLN1 alone could promote vascular repair and recovery and rejuvenate the aged vasculature for regeneration and repair and, thus, promote recovery and survival of aged mice. In some embodiment, FOXM1 expression was not induced in lungs of elderly COVID-19 patients which was in contrast to FOXM1 induction in lungs of mid-age adult COVID-19 patients. These data for the first time provide unequivocal evidence that these drugs or their combination can be used for effective treatment of elderly patients with COVID-19 and COVID-19 associated respiratory distress and multi-organ failure, sepsis, ARDS, and multi-organ failure as well as critical limb ischemia and restenosis.

The presently disclosed subject matter is described herein using several definitions, as set forth below and throughout the application.

### Definitions

Unless defined otherwise, all technical and scientific terms used herein have the same meaning as commonly understood by one of skill in the art to which the invention pertains. Although any methods and materials similar to or equivalent to those described herein can be used in the practice or testing of the present invention, the preferred methods and materials are described herein.

Unless otherwise specified or indicated by context, the terms “a”, “an”, and “the” mean “one or more.” For example, “a component” should be interpreted to mean “one or more components.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of” while encompassed by the term “comprising,” should be interpreted as a “partially closed” transitional term which permits additional elements succeeding this transitional term, but only if those additional elements do not materially affect the basic and novel characteristics of the claim.

Ranges recited herein include the defined boundary numerical values as well as sub-ranges encompassing any non-recited numerical values within the recited range. For example, a range from about 0.01 mM to about 10.0 mM includes both 0.01 mM and 10.0 mM. Non-recited numerical values within this exemplary recited range also contemplated include, for example, 0.05 mM, 0.10 mM, 0.20 mM, 0.51 mM, 1.0 mM, 1.75 mM, 2.5 mM 5.0 mM, 6.0 mM, 7.5 mM, 8.0 mM, 9.0 mM, and 9.9 mM, among others. Exemplary sub-ranges within this exemplary range include from about 0.01 mM to about 5.0 mM; from about 0.1 mM to about 2.5 mM; and from about 2.0 mM to about 6.0 mM, among others.

The terms “subject” and “patient” are used interchangeably herein. The subject treated by the presently disclosed methods, uses, and compositions is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, infant, juvenile, adult, and elderly (about 50, about 55, about 60, about 65, about 70 or about 75 years old or older). In some embodiments, an elderly human subject is about 60 years old or older. Further, a “subject” can include a patient diagnosed with or suspected of having a condition or disease.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy.

In general, the “effective amount” or “therapeutically effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the composition of any encapsulating matrix, the target tissue, the subject's overall condition, and the like.

As used herein the term “analogue” or “functional analogue” refer to compounds having similar physical, chemical, biochemical, or pharmacological properties. Functional analogues are not necessarily structural analogues with a similar chemical structure. An example of pharmacological functional analogues are morphine, heroine, and fentanyl, which have the same mechanism of action, but fentanyl is structurally quite different from the other two. Exemplary analogues of DMOG include, but are not limited to roxadustat, molidustat, vadadustat, and desidustat. Exemplary analogues of Thienopyridine include, but limited to Apocynin, Ebselen, and NOX2ds-tat. These are functional analogues as they all have NOX2 inhibiting activity.

The term “combination therapy” is used in its broadest sense and means that a subject is administered at least two agents. More particularly, the term “in combination” with respect to therapy administration refers to the concomitant administration of two (or more) active agents for the treatment of a disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms.

Further, the presently disclosed compositions can be administered alone or in combination with adjuvants that enhance stability of the agents, facilitate administration of pharmaceutical compositions containing them in certain embodiments, provide increased dissolution or dispersion, increase activity, provide adjuvant therapy, and the like, including other active ingredients. In some embodiments, such combination therapies utilize lower dosages of the conventional therapeutics, thus avoiding possible toxicity and adverse side effects incurred when those agents are used as monotherapies.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of an agent and at least one additional therapeutic agent is greater than the sum of the biological activities of the respective agents when administered individually.

As used herein, “genetic therapy” or “gene therapy” involves the transfer of heterologous DNA to the certain cells, target cells, of a mammal, particularly a human, with a disorder or conditions for which therapy or diagnosis is sought. The DNA is introduced into the selected target cells in a manner such that the heterologous DNA is expressed and a therapeutic product encoded thereby is produced. In some embodiments, the heterologous DNA, directly or indirectly, mediates expression of DNA that encodes the therapeutic product. In some embodiments, the heterologous DNA encodes a product, such as a peptide or RNA that mediates, directly or indirectly, expression of a therapeutic product. In some embodiments, genetic therapy is used to deliver a nucleic acid encoding a gene product to replace a defective gene or supplement a gene product produced by the mammal or the cell in which it is introduced. In some embodiments, the introduced nucleic acid encodes a therapeutic compound, such as a growth factor or inhibitor thereof, or a signaling molecule, a transcription factor, etc. that is not generally produced in the mammalian host, or the host cell, or that is not produced in therapeutically effective amounts or at a therapeutically useful time. In some embodiments, the introduced nucleic acid encodes a therapeutic compound, such as an antisense oligo, a small interfering RNA, a guide RNA oligo. In some embodiments, the heterologous DNA encoding the therapeutic product is modified prior to introduction into the cells of the afflicted host in order to enhance or otherwise alter the product or expression thereof.

As used herein, “heterologous nucleic acid sequence” is generally DNA that encodes RNA and proteins that are not normally produced in vivo by the cell in which it is expressed For example, the heterologous nucleic acid may include encode a gene product that is not typically expressed in the host organism, or in the host cell, or may encode a gene product that is not expressed by the host organism or the host cell at particular time, at a particular stage of development, or under particular conditions the host or the host cell is currently experiencing. In some embodiments, a heterologous nucleic acid sequence mediates or encodes mediators that alter the expression of endogenous genes by affecting transcription, translation, or other regulatable biochemical processes. Any DNA that one of skill in the art would recognize or consider as heterologous or foreign to the cell in which it is expressed is herein encompassed by heterologous DNA. Examples of heterologous DNA include, but are not limited to, native or non-native DNA that encodes traceable marker proteins, such as a protein that confers drug resistance, DNA that encodes therapeutically effective substances, such as anti-cancer agents, enzymes, transcription factors, signaling molecules, receptors, and hormones, and DNA that encodes other types of proteins, such as antibodies.

### Therapeutic Compositions, Formulations, and Modes of Administration

Disclosed herein are compositions and methods useful for the treatment of COVID-19 and COVID-19-associated respiratory distress and multi-organ failure, acute respiratory distress syndrome (ARDS), sepsis, critical limb ischemia, and restenosis in a subject in need thereof. The methods disclosed herein include administering one or more therapeutic compositions to a subject in need thereof.

In some embodiments, the compositions of the present disclosure include one or more compounds that inhibits endothelial injury and inflammation. In some embodiments, the compositions include one or more compounds that promote endothelial regeneration and vascular repair. In some embodiments, the compounds include a combination of (a) one or more compounds that inhibit endothelial injury and inflammation, and (b) one or more compounds that promote endothelial regeneration and vascular repair.

By way of example, but not by way of limitation, compounds that inhibits endothelial injury and inflammation include, but are not limited to N-acetyl cysteine (NAC), NOX2 inhibitors (Thienopyridine, NOX2ds-tat), pan-NOX inhibitors (Apocynin, Ebselen, APX-115), Reseveratrol (trans-E-resveratrol, “RV”) nanoparticles and analogues thereof (e.g., RV-loaded poly(D,L-lactic-co-glycolic acid) (PLGA) nanoparticles coated with long linker poly(ethylene glycol) (PEG), and RV-loaded poly(D,L-lactic acid) (PLA) nanoparticles coated with long linker PEG), and NOX2 inhibiting nucleic acid.

By way of example, but not by way of limitation, compounds that promote endothelial regeneration and vascular repair include, but are not limited to Decitabine (e.g. Dacogen, INQOVI) and its analogues (e.g., Vidaza, ONUREG), dimethyoxalylglycine (DMOG, a prolyl hydroxylase (PHD) inhibitor) and analogs thereof (e.g., roxadustat (FG-4592), molidustat, vadadustat, and desidustat), Sirtuinl inhibitors (e.g., Selisistat, AG1031) and SIRT1 inhibiting nucleic acid, rabeprazol (e.g., Aciphex) and its analogues, phenazopyridine and its analogues; EGLN1 inhibiting nucleic acid, SIRT1 inhibiting nucleic acid, HIF1A expressing nucleic acid, FOXM1 expressing nucleic acid.

Thus the compounds (drugs) exemplified above and their analogs can be repurposed or used for treatment of COVID-19 and COVID-19 associated respiratory distress and multi-organ failure, sepsis, ARDS, and multiple organ failure in aging patients or adult patients by either monotherapy or combination therapy.

As used herein the term Resveratrol refers to a compound having the formula C₁₄H₁₂O₃, and is represented by the chemical structure:

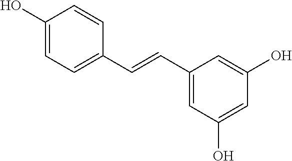 [SMILES: Oc1ccc(/C=C/c2cc(O)cc(O)c2)cc1]

Resveratrol has been reported to have anti-inflammatory, anti-oxidant and anti-cancer properties. However, its use is widely hindered by its poor solubility. The present invention identifies specific formulation with nanoparticles for treatment of COVID-19 respiratory distress and multi-organ failure, sepsis, and ARDS in patients. The nanoparticles include, but not limited to poly(D,L-lactic-co-glycolic acid) (PLGA)-b-poly(ethylene glycol) (PEG) copolymer, and poly(D,L-lactic acid) (PLA)-b-PEG copolymer. The molecular weight of PLGA is 5,000 to 100,000 Da, e.g., 55,000 Da; the molecular weight of PLA is 5,000-50,000 Da, e.g. 10,000 Da. The molecular weight of PEG is 1,000-10,000. The present invention found PEG2,000 Da (PEGl) is particularly useful. The nanoparticles include but limited to PLGA-b-PEG co-polymer, e.g., PLGA25,000-b-PEG2,000 and PLA-PEG copolymer, e.g., PLA10,000-b-PEG2,000. The estimated dose range is 0.05-50 mg/kg, e.g., 0.4 mg/kg in patients.

As used herein the term N-Acetylcysteine (NAC) also known as Acetylcysteine refers to a compound having the formula C₅H₉NO₃S, and is represented by the chemical structure:

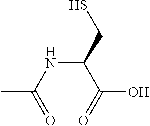

NAC is a drug for treatment of paracetamol overdose and thick mucus in patients with cystic fibrosis or chronic obstructive pulmonary disease. The present invention identifies new indication in treating COVID-19 respiratory distress and multi-organ failure, ARDS, and sepsis as a monotherapy or combination therapy.

As used herein the term Apocynin refers to a compound having the formula C₉H₁₀O₃, and is represented by the chemical structure:

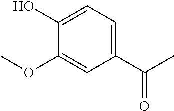

As used herein the term Ebselen refers to a compound having the formula C₁₃H₉NOSe, and is represented by the chemical structure:

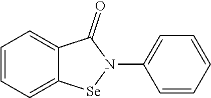 [SMILES: O=c1c2ccccc2[se]n1-c1ccccc1]

As used herein the term Thienopyridine refers to a compound having the formula C₇H₅NS, and is represented by the chemical structure:

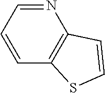

Apocynin also known as acetovanillone is a natural organic compound structurally related to vanillin. It functions as a NOX inhibitor and anti-oxidant. Ebselen is an organoselenium compound. It is a NOX inhibitor and anti-oxidant. Thienopyridine is an NOX2 inhibitor and also inhibits ADP receptor/P2Y12 and thus is used for its anti-platelet activity. The present invention employs their NOX 2 inhibiting activity and/or anti-oxidant activity for treatment of COVID-19 respiratory distress and multi-organ failure, ARDS and sepsis in elderly patients.

As used herein the term decitabine refers to a compound having the formula C₈H₁₂N₄O₄, and is represented by the chemical structure:

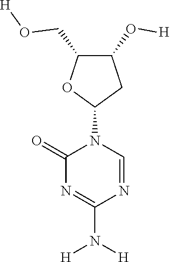 [SMILES: Nc1ncn([C@H]2C[C@@H](O)[C@@H](CO)O2)c(=O)n1]

Decitabine is a cytidine antimetabolite analogue with potential antineoplastic activity. Decitabine has been shown to incorporate into DNA and inhibit DNA methyltransferase, resulting in hypomethylation of DNA and intra-S-phase arrest of DNA replication. Decitabine is also known as 5-Aza-2′-deoxycytidine, Dacogen, and 5-Azadeoxycytidine. The present invention identifies new indication in treating COVID-19 respiratory distress and multi-organ failure, ARDS, and sepsis in aged subjects, such as age ≥60 years old. The estimated dosage range is 0.01-1 mg/kg, e.g., 0.02 mg/kg in patients. In some embodiments, Decitabine may be more effective on elderly subjects as compared to younger subjects.

As used herein, rabeprazol refers to a compound having the formula C₁₈H₂₁N₃O₃S and represented by the structure:

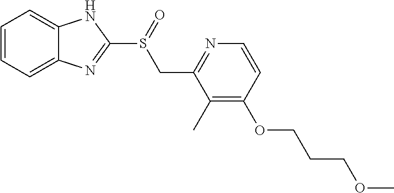 [SMILES: COCCCOc1ccnc(CS(=O)c2nc3ccccc3[nH]2)c1C]

Rabeprazole is a proton pump inhibitor that decreases the amount of acid produced in the stomach. Rabeprazole is used short-term to treat symptoms of gastroesophageal reflux disease (GERD) in adults and children who are at least 1 year old. Rabeprazole is used only in adults to treat conditions involving excessive stomach acid, such as Zollinger-Ellison syndrome. Rabeprazole is also used in adults to promote healing of duodenal ulcers or erosive esophagitis (damage to esophagus caused by stomach acid). Raberazole is also known as Aciphex, Habeprazole and Pariets. The present invention identifies new indication in treating COVID-19, ARDS, and sepsis as well as restenosis following PCI, and critical limb ischemia in subjects. The estimated dosage range is 0.5-10 mg/kg, e.g., 1.6 mg/kg in patients.

As used herein, the term phenazopyridine refers to a compound having the formula C₁₂H₁₂ClN₅ and represented by the structure:

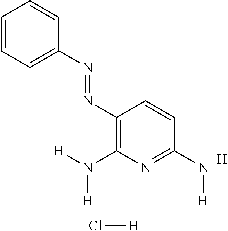

Phenazopyridine is often used to relieve the symptoms of urinary tract infections. Phenazopyridine is also known as phenazopyridine hydrochloride, phenazopyridine HCl, pyridium, and urodine. The present invention identifies new indication for Phenazopyridine and its analogues in treating COVID-19 respiratory distress and multi-organ failure, ARDS, and sepsis as well as anemia, restenosis following PCI, and critical limb ischemia in aged subjects, such as age ≥60 years old. The estimated dosage range is 1-20 mg/kg, twice a day, e.g., 4 mg/kg twice a day in patients. It is particularly useful in patients at age ≥60 years old.

As used herein dimethyoxalylglycine (DMOG) refers to a compound having the formula C₆H₉NO₅ and represented by the structure:

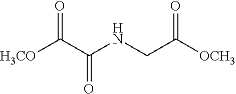 [SMILES: COC(=O)CNC(=O)C(=O)OC]

Exemplary analogues of DMOG include, but are not limited to roxadustat (FG-4592), molidustat, vadadustat, and desidustat. These drugs are current under clinical trials for treatment of anemia associated with kidney failure patients. The present invention identifies new indication in treating COVID-19 respiratory distress and multi-organ failure, ARDS, and sepsis as well as restenosis following PCI, and critical limb ischemia in subjects, it is particularly useful in patients at age ≥60 years old. The estimated roxadustat dosage range is 0.2-20 mg/kg, e.g., 2 mg/kg in patients.

As used herein Selisistat (EX-527) refers to a compound having the formula C13H13ClN2O and represented by the structure:

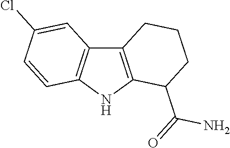 [SMILES: NC(=O)C1CCCc2c1[nH]c1ccc(Cl)cc21]

Selisistat is a Sirtuin 1 (SIRT1)-selective inhibitor. does not inhibit histone deacetylase (HDAC) or other sirtuin deacetylase family members (IC50 values are 98, 19600, 48700, >100000 and >100000 nM for SIRT1, SIRT2, SIRT3, HDAC and NADase respectively). Enhances p53 acetylation in response to DNA damaging agents. The present invention identifies new indication of Selisistat and its analogues in treating COVID-19 respiratory distress and multi-organ failure, ARDS, and sepsis as well as restenosis following PCI, and critical limb ischemia in subjects, it is particularly useful in patients at age ≥60 years old. The estimated dosage range is 0.1-6 mg/kg, e.g., 0.6 mg/kg in patients.

The compounds (drugs) disclosed herein can be used as monotherapy or combination therapy. For example, two or three drugs can be combined in the same dosage or different dosages, respectively. The compounds can be administered to a subject with the same schedule or different schedules via the same route of administration or different route of administration. Exemplary combination therapy includes for example, at least one compound that promotes endothelial regeneration and vascular repair, and at least one compound that inhibits endothelial injury and inflammation.

Exemplary combinations include, but are not limited to e.g., 1) Dexamethasone, with one or more of Decitabine (e.g., Dacogen, INQOVI, Vidaza, ONUREG), NAC, Apocynin, Selisistat, AG-1031, rabeprazol, phenazopyridine, roxadustat, molidustat, vadadustat, desidustat, SIRT1 inhibiting nucleic acid, EGLN1 inhibiting nucleic acid, HIF1A expressing nucleic acid, FOXM1 expressing nucleic acid; 2) NAC, with one or more of Decitabine (e.g., Dacogen, INQOVI, Vidaza, ONUREG), Selisistat, AG-1031, rabeprazol, phenazopyridine, roxadustat, molidustat, vadadustat, desidustat, SIRT1 inhibiting nucleic acid, EGLN1 inhibiting nucleic acid, HIF1A expressing nucleic acid, FOXM1 expressing nucleic acid; 3) Apocynin with one or more of Decitabine (e.g., Dacogen, INQOVI, Vidaza, ONUREG), Selisistat, AG-1031, rabeprazol, phenazopyridine, roxadustat, molidustat, vadadustat, desidustat, SIRT1 inhibiting nucleic acid, EGLN1 inhibiting nucleic acid, HIF1A expressing nucleic acid, FOXM1 expressing nucleic acid; 4) Thienopyridine with one or more of Decitabine (e.g., Dacogen, INQOVI, Vidaza, ONUREG), Selisistat, AG-1031, rabeprazol, phenazopyridine, roxadustat, molidustat, vadadustat, desidustat, SIRT1 inhibiting nucleic acid, EGLN1 inhibiting nucleic acid, HIF1A expressing nucleic acid, FOXM1 expressing nucleic acid; 5) Ebselen with one or more of Decitabine (e.g., Dacogen, INQOVI, Vidaza, ONUREG), Selisistat, AG-1031, rabeprazol, phenazopyridine, roxadustat, molidustat, vadadustat, desidustat, SIRT1 inhibiting nucleic acid, EGLN1 inhibiting nucleic acid, HIF1A expressing nucleic acid, FOXM1 expressing nucleic acid; 6) APX-115 with one or more of Decitabine (e.g., Dacogen, INQOVI, Vidaza, ONUREG), Selisistat, AG-1031, rabeprazol, roxadustat, molidustat, vadadustat, desidustat; 7) NOX2 inhibiting nucleic acid with one or more of Decitabine (e.g., Dacogen, INQOVI, Vidaza, ONUREG), Selisistat, AG-1031, rabeprazol, phenazopyridine, roxadustat, molidustat, vadadustat, desidustat, SIRT1 inhibiting nucleic acid, EGLN1 inhibiting nucleic acid, HIF1A expressing nucleic acid, FOXM1 expressing nucleic acid; 8) NOX2ds-tat with one or more of Decitabine (e.g., Dacogen, INQOVI, Vidaza, ONUREG), Selisistat, AG-1031, rabeprazol, phenazopyridine, roxadustat, molidustat, vadadustat, desidustat, SIRT1 inhibiting nucleic acid, EGLN1 inhibiting nucleic acid, HIF1A expressing nucleic acid, FOXM1 expressing nucleic acid; 9) RV nanoparticles with one or more of Decitabine, AG-1031, rabeprazole, phenazopyridine, roxadustat, molidustat, vadadustat, desidustat, SIRT1 inhibiting nucleic acid, EGLN1 inhibiting nucleic acid, HIF1A expressing nucleic acid, FOXM1 expressing nucleic acid.

Additionally or alternatively, in some embodiments, viral or non-viral (e.g., nanoparticle, liposome) delivery of FOXM1, or HIF1A alone or combination with one or more of Dexamethasone, NAC or NOX inhibitor including Thienopyridine/Apocynin/Ebselen/APX-115/NOX2ds-tat, or NOX2 inhibiting nuclei acid is also useful for treatment. Viral or non-viral (e.g. Nanoparticle) delivery of SIRT1 inhibiting nucleic acid, or EGLN1 inhibiting nucleic acid alone or combination with either Dexamethasone or NAC or NOX inhibitor including Thienopyridine/Apocynin/Ebselen/APX-115/NOX2ds-tat, or RV is useful for treatment. Viral or non-viral (e.g. Nanoparticle) delivery of NOX2 inhibiting nucleic acid alone or combination with either Selisistat, and/or AG-1031, and/or rabeprazol and/or Decitabine (e.g., Dacogen, INQOVI, Vidaza, ONUREG), phenazopyridine and/or roxadustat/molidustat/vadadustat/desidustat is also useful for treatment.

Also disclosed herein are therapeutic compositions comprising a combination of one or more of decitabine and/or analogues thereof, rabeprazol and/or analogues thereof, phenazopyridine and/or analogues thereof, roxadustat and/or analogues thereof (e.g., molidustat/vadadustat/desidustat), Selisistat and/or Sirtuinl inhibitors (e.g. AG1031), NAC, Dexamethasone, Thienopyridine, NOX2ds-tat, Apocynin, Ebselen, APX-115, NOX2 inhibitors, and RV nanoparticles, for the treatment of COVID-19, COVID-19 sepsis, COVID-19 respiratory distress and multi-organ failure, sepsis, and ARDS by inhibiting vascular injury and/or promoting vascular repair and rejuvenation in aging patients.

In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams and Wilkins (2000).

Use of pharmaceutically acceptable inert carriers to formulate the compounds herein disclosed for the practice of the disclosure into dosages suitable for administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, dragees, liquids, gels, syrups, slurries, suspensions, and the like, for oral ingestion. For nasal or inhalation delivery, the compositions of the disclosure also may be formulated by methods known to those of skill in the art, and may include, for example, but not limited to, sprays, inhalers, vapors; solubilizing, diluting, or dispersing substances, such as saline, preservatives, such as benzyl alcohol; absorption promoters; and fluorocarbons may be included.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.1 to 1000 mg, from 0.5 to 200 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, and the preference and experience of the attending physician.

In some embodiments, the pharmaceutical composition comprises a therapeutic nucleic acid. By way of example, but not by way of limitation, in some embodiments, nucleic acid compositions are administered to a subject via delivery methods including viral vectors, liposomes, nanoparticles, or naked nucleic acids, such as naked DNA.

In some embodiments, therapeutic nucleic acids are provided as inhibitory RNA oligonucleotides and include, but are not limited to modified or unmodified antisense oligonucleotides, small interfering RNAs (siRNA), guide RNA oligonucleotides, or a combination thereof, antisense, siRNA or guide RNA expressing plasmid DNA. By way of example, but not by way of limitation, exemplary therapeutic, inhibitory or inhibiting nucleic acids include NOX2 siRNA, Sirtuin 1 (SIRT1) siRNA, and EGLN1 siRNA.

In some embodiments, therapeutic nucleic acids are engineered and formulated to express a therapeutic protein after administration. By way of example, but not by way of limitation, exemplary therapeutic nucleic acids engineered and formulated to express a therapeutic protein include a FOXM1 expressing nucleic acid, and a HIF1A expressing nucleic acid

Thus, in some embodiments, the presently disclosed subject matter provides a pharmaceutical composition comprising one or more of rabeprazol, phenazopyridine, DMOG analogs (e.g., roxadustat, molidustat, vadadustat, desidustat), Selisistat, AG1031, decitabine (e.g., Dacogen, INQOVI, Vidaza, ONUREG), Dexamethasone, NAC, Apocynin, APX-115, Thienopyridine, NOX2ds-tat, Ebselen, and analogues thereof, and optionally additional agents, and a pharmaceutically acceptable carrier. Additionally or alternatively, in some embodiments, the presently disclosed subject matter provides a pharmaceutical composition comprising one or more of a NOX2 inhibiting nucleic acid, a FOXM1 expressing nucleic acid, a HIF-1α expressing nucleic acid, a Sirtuinl inhibiting nucleic acid, or an EGLN1 inhibiting nucleic acid.

Methods:

Embodiments of the technology include treatment methods whereby pharmaceutical compositions disclosed herein (e.g., a composition including one or more of (a) a compound that inhibits endothelial injury and inflammation, and (b) a compound that promotes endothelial regeneration and vascular repair) are administered to a subject in need thereof.

In some embodiments, a subject in need thereof is a subject who has been diagnosed with or is at risk of having COVID-19. In some embodiments, a subject in need thereof includes a subject suffering from one or more COVID-19 related symptoms, including but not limited to: COVID-19-related sepsis, and COVID-19-related respiratory distress and organ failure.

In some embodiments, a subject in need thereof includes a subject suffering from sepsis, acute respiratory distress syndrome (ARDS), acute inflammatory injury, and infection-induced organ failure characterized by increased lung microvascular permeability and inflammation.

In some embodiments, a subject in need thereof includes a subject suffering from cardiovascular diseases including restenosis, and peripheral vascular disease, e.g., critical limb ischemia.

As noted above, the compositions of the present disclosure may be formulated for a desired mode of administration, including but not limited to parenterally, orally, and via inhalation.

In some embodiments of the methods, a composition may be administered a single time, or may be administered multiple times, over the course of one or more days or weeks.

In some embodiments, a subject in need thereof is elderly, e.g., 60 years old or older, or 70 years old or older, or, 80 years old or older, 90 years old or older In some embodiments, the subject is a human.

In some embodiments, the subject is a non-human mammal.

In some embodiments, the methods include administering one or more of the pharmaceutical compositions described herein to a subject of any age. In some embodiments, the methods include administering one or more of the pharmaceutical compositions described herein to an elderly subject. Useful, maybe more effective, or may have a greater therapeutic effect when administered to by way of example only, but not by way of limitation, in some embodiments, compositions comprising Dexamethasone, Resveratrol, NAC, rabeprazole, phenazopyridine, roxadustat, molidustat, vadadustat, and desidustat, EGLN1 inhibiting nucleic acid, HIF1A expressing nucleic acid, FOXM1 expressing nucleic acid may be administered to a subject of any age, with an expectation of a positive therapeutic effect. In some embodiments, by way of example only and not by way of limitation, compositions comprising Decitabine, Apocynin, Ebselen, APX-115, NOX2 inhibiting peptide (NOX2ds-tat), Thienopyridine, Selisistat, and AG-1031, NOX2 inhibiting nucleic acid, SIRT1 inhibiting nucleic acid may be particularly useful in elderly subjects, e.g., subjects at least about 60 years old or older. That is, compositions comprising these exemplary compounds have a greater therapeutic effect on an elderly subject in need thereof as compared to a non-elderly subject (e.g., a teen or someone under about 60 years old, for example).

Applications

Exemplary application of the methods and compositions disclosed herein include but are not limited to the following: (1) treatment of COVID-19 and COVID-related conditions including, but not limited to (2) COVID-related respiratory distress and multi-organ failure in aging patients and also adult patients, treatment of COVID-related sepsis, and septic shock in aging patients and also adult patients; (3) treatment of acute respiratory distress syndrome in aging patients and also adult patients; (4) treatment of sepsis and multiple organ failure associated with sepsis in aging patients and also adult patients; (5) treatment of acute inflammation in aging patients and also adult patients; (6) treatment of restenosis in aging patients and also adult patients; (7) treatment of peripheral ischemic vascular disease (e.g., critical limb ischemia) in aging patients and also adult patients. In some embodiments, one or more of decitabine (e.g. Dacogen, INQOVI) and its analogues (e.g., Vidaza, ONUREG), N-acetyl cysteine (NAC), NOX2 inhibitors (Thienopyridine, NOX2ds-tat,), pan-NOX inhibitors (Apocynin, Ebselen, APX-115), Reseveratrol (trans-E-resveratrol, “RV”) nanoparticles and analogues thereof (e.g., RV-loaded nanoparticles comprising of poly(D,L-lactic-co-glycolic acid) (PLGA)-b-long linker poly(ethylene glycol) (PEG) copolymer, and RV-loaded nanoparticles comprising of poly(D,L-lactic acid) (PLA)-b-long linker PEG copolymer), and NOX2 inhibiting nucleic acid, and one or more of a prolyl hydroxylase (PHD) inhibitor) and DMOG analogs (e.g., roxadustat, molidustat, vadadustat, and desidustat), Sirtuinl inhibitors (e.g., Selisistat and its analogues, AG1031), rabeprazol (e.g., Aciphex) and its analogues, phenazopyridine (e.g., Pyridium) and its analogues; and SIRT1 inhibiting nucleic acid, EGLN1 inhibiting nucleic acid, HIF-1α expressing nucleic acid, FOXM1 expressing nucleic acid is administered to a subject suffering from one or more of the aforementioned diseases. In some embodiments, the subject is an adult human, and in some embodiments the subject is an elderly human, e.g., age 60 years old or older.

Acute respiratory distress syndrome (ARDS) is a form of acute-onset hypoxemic respiratory failure with bilateral pulmonary infiltrates, which is caused by acute inflammatory edema of the lungs not attributable to left heart failure. The most common underlying causes of ARDS include sepsis, severe pneumonia, inhalation of harmful substance, burn, major trauma with shock, as well as viral infection. Endothelial injury characterized by persistently increased lung microvascular permeability resulting in protein-rich lung edema is a hallmark of ARDS. Despite recent advances on the understanding of the pathogenesis, there are currently no effective pharmacological or cell-based treatment of the disease with a mortality rate as high as 40%. Compared to young adult patients, the incidence of ARDS resulting from sepsis, pneumonia, flu in elderly patients (≥60 yr) is as much as 19-fold greater and the mortality rate is 10-20-fold greater (1, 8-12). However, the underlying causes are poorly understood. Also crucially little is known how aging influences mechanisms of endothelial regeneration and resolution of inflammatory lung injury.

COVID-19 caused by SARS-CoV2 infection is considered as a systemic disease that primarily injures the vascular endothelium although the portal for the virus is inhalational. Clinically, soon after onset of respiratory distress from COVID-19, patients develop severe hypoxiemia, and interstitial rather than alveolar edema. Pathological examinations reveal that the lungs have extensive hemorrhages and are expanded with exudatives with high incidence of thrombi in small vessels, pointing to excessive vascular endothelium injury. In addition to respiratory distress, cardiovascular complication with widespread macro and micro-thromboses is another feature of severe COVID-19. The morbidity and mortality of COVID-19 patients in elderly patients are much greater than that in adult patients. In New York city, the death rates of COVID-19 patients are 168, 1540, 5020, and 12630 per million people in age group of 18-44, 45-64, 65-74, and ≥75 years old, respectively. In Italy, the mortality rate of COVID-19 patients at age of 20-39 years is less than 0.3%, 10.1% for 60-69 years old COVID-19 patients while more than 25% for ≥70 years old COVID-19 patients.

In some embodiments, one or more of the aforementioned conditions or diseases is caused by infection, or is exacerbated by infection, which may be bacterial or viral in origin.

Exemplary, non-limiting examples of viral infections and viral agents include influenza, pneumonia, the common cold (e.g., mainly caused by rhinovirus, coronavirus, and adenovirus) encephalitis and meningitis, (e.g., caused by enterovirus and herpes virus), Zika virus, HIV, hepatitis C, polio, Dengue fever, H1N1 swine flu, Ebola, MERS-CoV, SARS virus, SARS-CoV2 (causing COVID-19), and other coronavirus, mumps, human papillomavirus, herpes virus, rotavirus and chicken pox.

Exemplary, non-limiting examples of bacterial infections and bacterial agents include pneumonia, tuberculosis, typhoid, typhus, meningitis, upper respiratory tract infections, eye infections, sinusitis, urinary tract infections, skin infections, and nosocomial infections. These are caused by either gram negative or positive bacterial infections.

In some embodiments, the subject is treated according to the methods of the present disclosure when an infection has been identified or is suspected, but prior to the onset of sepsis, septic shock, ARDS, COVID-19 respiratory distress, respiratory failure or multiple organ failure due to sepsis or infection, etc. Accordingly, the compositions and methods of the present disclosure may be employed prophylactically as well as therapeutically.

Advantages

Current therapies for COVID-19 respiratory distress and multi-organ failure, sepsis and ARDS are merely supportive; there are no effective therapies for these conditions in adult patients, and particularly in aging patients who have much greater morbidity and mortality. Persistent endothelial injury is a prominent feature of these conditions, in particular, COVID-19 is now considered as a systemic disease that primarily injures the vascular endothelium. In contrast the methods and compositions of the present disclosure provide therapeutic relief by, for example, inhibiting injury, especially vascular injury, and cytokine storm, promoting vascular survival, repair, and recovery, and also inhibiting injury and promoting repair and recovery by combination therapy, and therefore fill a much-needed gap in the treatment of these diseases and conditions. Moreover, while the therapies disclosed herein are effective and safe for patients of all age groups, they are surprisingly and unexpectedly potentially more effective in elderly patients than younger patient.

### Examples

The following Examples are illustrative and are not intended to limit the scope of the claimed subject matter.

Example 1. Therapeutic activation of endothelial regeneration, vascular repair and resolution of inflammation in elderly patients with COVID-19 and COVID-19 respiratory distress and multi-organ failure, sepsis, ARDS, and multi-organ failure, as well as cardiovascular diseases including but not limited to restenosis and critical limb ischemia.

Rationale: Aging is a risk factor of high incidence and great morbidity and mortality of COVID-19 respiratory distress and multi-organ failure, sepsis and ARDS. However, it is unknown how aging influences mechanisms of endothelial regeneration and resolution of inflammatory lung injury.

Objectives: We aimed to investigate the underlying mechanisms and explore therapeutic approach to reactivate vascular repair and resolve inflammatory injury in aged lungs.

Methods. Genetic lineage tracing was used to study endothelial regeneration. Sepsis was induced by either cecal ligation and puncture (CLP) or lipopolysaccharide (LPS). Vascular permeability and inflammation was measured. In vivo BrdU labeling was used to quantify endothelial proliferation. Foxm1 transgenic mice and gene transduction of FoxM1 in lungs of aged mice were used. FDA-approved drug library was screened to identify drugs which could rejuvenate the aged endothelium for regeneration and repair. Autopsy lung samples from COVID-19 patients were employed to validate clinical relevance of our findings in animals.

Measurements and Main Results. Endothelial regeneration was mediated by lung resident endothelial proliferation, which was impaired in aged mice. Aged mice exhibited persistent inflammatory lung injury and great mortality following sepsis challenge. Expression of FoxM1, an important mediator of lung endothelial regeneration in young adult mice, was not induced in aged lungs. Transgenic expression of FoxM1 normalized vascular repair in aged mice and promoted survival following sepsis challenge. In vivo gene transduction of FOXM1 targeting vascular endothelium or repurposing treatment with FDA-approved drug Decitabine was sufficient to reactivate FoxM1-dependent endothelial regeneration in aged mice, reverse aging-impaired resolution of inflammatory injury, and promote survival. In COVID-19 lung autopsy samples, FOXM1 expression was not induced in vascular endothelial cells of elderly patients in contrast to mid-age patients, validating the clinical relevance of the findings in aged mice.

Conclusion. These results show that aging impairs intrinsic endothelial regeneration and vascular repair leading to persistent inflammatory lung injury following sepsis challenge, and therapeutic restoration of FoxM1 expression can reactivate vascular repair and resolution of inflammatory injury in aged mice. Thus, activation of FoxM1-mediated endothelial regeneration and vascular repair represents a potential effective approach for treatment of COVID-19, COVID-19 sepsis, COVID-19 respiratory distress and organ failure, sepsis, ARDS, and multi-organ failure in elderly patients with pneumonia, flu, SARS-CoV2, and other pathological conditions.

Introduction

Acute respiratory distress syndrome (ARDS) is a form of acute-onset hypoxemic respiratory failure with bilateral pulmonary infiltrates, which is caused by acute inflammatory edema of the lungs not attributable to left heart failure (1-3). The most common underlying causes of ARDS include sepsis, severe pneumonia, inhalation of harmful substance, burn, and major trauma with shock. Severe COVID-19 results in severe sepsis, respiratory distress and multi-organ failure. Endothelial injury characterized by persistently increased lung microvascular permeability resulting in protein-rich lung edema is a hallmark of severe COVID-19 including COVID-19 sepsis, COVID-19 respiratory distress and multi-organ failure, severe sepsis and ARDS (4-7). Despite recent advances on the understanding of the pathogenesis, there are currently no effective pharmacological or cell or gene-based treatment of COVID-19, sepsis and ARDS with a mortality rate as high as 40% (1-3). Compared to young adult patients, the incidence of COVID-19 respiratory distress and multi-organ failure and ARDS resulting from sepsis, pneumonia, flu, and COVID-19 in elderly patients (≥60 yr) is as much as 19-fold greater and the mortality rate is 10-100 fold greater (1, 8-12). However, the underlying causes are poorly understood. Also crucially little is known how aging influences mechanisms of endothelial regeneration and resolution of inflammatory lung injury.

The forkhead box (Fox) transcriptional factors share homology in the winged helix or forkhead DNA-binding domains (13, 14). Among the Fox family, FoxM1 is the first one identified as a proliferation-specific transcriptional factor. FoxM1 is expressed during cellular proliferation and mediates cell cycle progression by transcriptional control of many of the cell cycle genes (15-19). During embryogenesis, FoxM1 is expressed in many types of cells, such as cardiomyocytes, endothelial cells (ECs), hepatocytes, lung epithelium cells, and smooth muscle cells (20-23). In adult mice, FoxM1 is restrictively expressed in intestinal crypts, thymus and testes (15, 16). Although FoxM1 is silenced in terminally differentiated cells (15-17), it can be induced after organ injury. We have reported that FoxM1 is induced in lung ECs in the repair phase but not in the injury phase following sepsis challenge (24). In EC-restricted Foxm1 null mice, pulmonary vascular EC proliferation and endothelial barrier recovery are defective following inflammatory lung injury (24). FoxM1 also mediates re-annealing of the endothelial adherens junctional complex to restore the endothelial barrier function following vascular injury (25). Additionally, we also showed that EC-expressed FoxM1 is the endogenous mediator of exogenous stem/progenitor cells-elicited paracrine effects on vascular repair and resolution of inflammatory lung injury (26). These results demonstrate the critical role of FoxM1 in vascular repair. Other studies also demonstrate the important role of FoxM1 in mediating lung epithelial repair (27) and hepatocyte regeneration (28) after injury in adult mice. Thus, FoxM1 is an important reparative transcription factor. However, it is unknown if FoxM1 can be induced in aged lungs and whether forced expression of FoxM1 in pulmonary vascular ECs is sufficient to reactivate vascular repair to resolve inflammatory lung injury in aged mice following sepsis challenge.

Here we sought to define the cell source of origin mediating endothelial regeneration and determine how aging affects this process as well as vascular repair and resolution of inflammatory lung injury. We further delineated the underlying molecular mechanisms. Our studies demonstrate that aging impairs the intrinsic endothelial regeneration program and thus vascular repair and inflammation resolution. Restored FoxM1 expression in lung ECs in aged mice is necessary and sufficient to re-activate lung endothelial regeneration and vascular repair and thereby resolve inflammatory lung injury and promote survival following sepsis challenge. Thus, therapeutic activation of FoxM1 expression in aged lungs by either repurposed FDA-approved drugs or nanoparticle delivery of FoxM1 gene represent a novel and effective treatment of COVID-19, and COVID-19 sepsis, COVID-19 respiratory distress and multi-organ failure, sepsis, septic shock, ARDS, and multi-organ failure in elderly patients to reduce morbidity and mortality.

Methods

Mice. EndoSCL-CreERT2/mTmG lineage tracing mice were generated by breeding the mice carrying a double-fluorescent reporter expressing membrane-targeted tandem dimer Tomato (mT) prior to Cre-mediated excision and membrane-targeted green fluorescent protein (mG) after excision (mTmG mice, #007676, the Jackson Laboratory) with EndoSCL-Cre^{ERT2} transgenic mice (29-31) (C57BL/6 background) containing tamoxifen-inducible Cre-ERT2 driven by the 5′ endothelial enhancer of the stem cell leukemia locus. Foxm1 transgenic

Foxm1^{Tg} mice were described previously (32, 33). Both male and female mice were used in the experiments. Mice at various ages (3-5 mo. old referred as young or adult; 19-21 mo. old referred as aged, 25 mo. old referred as elderly) were used. The experiments were conducted according to NIH guidelines on the use of laboratory animals. The animal care and study protocols were approved by the Institutional Animal Care and Use Committees of Northwestern University and The University of Illinois at Chicago.

Induction of lung injury. Polymicrobial sepsis was induced by CLP using a 23-gauge needle (34). Briefly, mice were anesthetized with inhaled isofluorane (2.5% mixed with oxygen). When the mice failed to respond to paw pinch, buprenex (0.1 mg/kg) was administered subcutaneously prior to sterilization of the skin with povidone iodine, then a midline abdominal incision was made. The cecum was exposed and ligated with a 4-0 silk tie placed 0.6 cm from the cecum tip, and the cecal wall was perforated with a 23-gauge needle. Control mice underwent anesthesia, laparotomy, and wound closure, but no cecal ligation or puncture. Following the procedure, 500 μl of prewarmed normal saline was administered subcutaneously. Within 5 min following surgery, the mice woke from anesthesia. The recovered mice subcutaneously received a second dose of buprenex at 8h post-surgery.

To induce endotoxemia, mice received a single dose of LPS (0.25-2.5 mg/kg BW, Escherichia coli 055:B5, Santa Cruz, St. Dallas, Tex.) by i.p. injection. The LPS dose was dependent on the aging of the mice (3-9 mo old, 2.5 mg/kg; 19-21 mo. old, 1.0 mg/kg; 25 mo. old, 0.25 mg/kg). All mice were anesthetized with ketamine/xylazine (100/5 mg/kg BW, i.p.) prior to tissue collection. For the survival study, mice were treated with a single dose of LPS (1.5 mg/kg, i.p.) and monitored for 7 days.

Vascular permeability assessment. The Evans blue dye-conjugated albumin (EBA) extravasation assay was performed as previously described (26, 34). Briefly, mice were retro-orbitally injected with EBA at a dose of 20 mg/kg BW at 30 minutes prior to tissue collection. Lungs were perfused free of blood with PBS, blotted dry and weighed. Next, lung tissues were homogenized in 1 ml PBS and incubated with 2 volumes of formamide at 60° C. for 18 hours. The homogenates were then centrifuged at 10,000×g for 30 minutes. The optical density of the supernatant was determined at 620 nm and 740 nm. The extravasated EBA in lung homogenate was presented as μg of Evans blue dye per g lung tissue.

Myeloperoxidase assay. Following perfusion free of blood, lung tissues were collected and homogenized in 50 mmol/L phosphate buffer. Homogenates were centrifuged at 15,000×g for 20 minutes at 4° C. The pellets were resuspended in phosphate buffer containing 0.5% hexadecyl trimethylammonium bromide and subjected to a cycle of freezing and thawing. Subsequently, the pellets were homogenized and the homogenates were centrifuged again. Absorbance was measured at 460 nm every 15 secs for 3 minutes and data expressed as ΔOD460/min/g lung tissue (26, 34).

Cell proliferation. At 8 h prior to tissue collection, BrdU (Sigma-Aldrich, St Louis, Mo.) was injected i.p. into mice at 50 mg/kg BW. Mouse lung cryosections were stained overnight with anti-BrdU (1:3, BD Biosciences, San Jose, Calif.) and incubated with Alexa Fluo 488-conjugated secondary antibody (1:200, Life Technologies, Grand Island, N.Y.). Lung vascular ECs were immunostained with anti-vWF (1:300, Sigma-Aldrich, St Louis, Mo.) and anti-CD31 (1:100, BD Biosciences, San Jose, Calif.) antibodies at 4° C. then the sections were incubated with Alexa Fluor 594-conjugated secondary antibodies (1:200, Life Technologies, Grand Island, N.Y.). The nuclei were counterstained with DAPI (Life Technologies, Grand Island, N.Y.). Three consecutive cryosections from each mouse lung were examined, the average number of BrdU+ nuclei was used (24, 34).

FACS analysis. After perfusion free of blood with PBS, lung tissues were cut into small pieces, and then incubated with 1 mg/ml collagenase A (Roche Applied Science) for 1 h at 37° C. in a shaking water bath (200 rpm). After digestion, the tissue was dispersed to a single cell preparation using the gentle MACS™ Dissociator (Miltenyi Biotec) with lung program 2. The cells were then filtered using a 40 μm Nylon cell strainer and blocked with 20% FBS for 30 min. After incubation with Fc blocker (1 μg/106 cells, BD Biosciences), the cells were immunostained with anti-CD45-PB (1:800, BioLegend) and/or anti-CD31-APC (1:600, BD Biosciences) for 45 min at room temperature. Cells were then analyzed by flow cytometry (Fortessa, BD Biosciences) and sorted by flow-assisted cell sorting (Moflo Asrtios machine, Beckman Coulter). mGFP- or tdTomato-labelled cells were directly analyzed with 488 nm or 561 nm laser wavelengths, respectively.

Molecular analysis. Total RNA was isolated using an RNeasy Mini kit including DNase I digestion (Qiagen, Valencia, Calif.). Following reverse transcription, quantitative RT-PCR analysis was performed using a sequence detection system (ABI ViiA 7 system; Life Technologies, Grand Island, N.Y.). The following primers sets were used for analysis: mouse FoxM1 primers, 5′-CACTTGGATTGAGGACCACTT-3′ (SEQ ID NO: 1) and 5′-GTCGTTTCTGCTGTGATTCC-3′ (SEQ ID NO: 2); mouse cyclophilin primers, 5′-CTTGTCCATGGCAAATGCTG-3′ (SEQ ID NO: 3) and 5′-TGATCTTCTTGCTGGTCTTGC-3′ (SEQ ID NO: 4). Primers for mouse Cdc25c, Ccna2, Ccnb1, Tnf, Il6, Nos2, and Icam1 were purchased from Qiagen. The mouse gene expression was normalized to cyclophilin.

Western blot analysis was performed using an anti-FoxM1 antibody (1:800, sc-376471, Santa Cruz Biotechnology, Santa Cruz) and the same blot was incubated with anti-β-actin antibody (1:3000, BD Biosciences, San Jose, Calif.) as a loading control.

Imaging. Following immunostaining, lung sections were imaged with a confocal microscope system (LSM510; Carl Zeiss, Inc) equipped with a 63×1.2 NA objective lens (Carl Zeiss, Inc.). For lineage tracing studies, the cryosections were directly mounted with Prolong Gold mounting media containing DAPI.

Histology. Lung tissues were fixed by 5 min instillation of 10% PBS-buffered formalin through tracheal catheterization at a trans-pulmonary pressure of 15 cm H2O, and then agitated overnight at room temperature. After paraffin processing, the tissues were sectioned (5 μm) and stained with hematoxylin and eosin.

Transduction of plasmid DNA into lung vascular endothelial cells in mice. To make liposomes, a mixture comprised of dimethyldioctadecylammonium bromide and cholesterol (1:1 molar ratio) was dried using a Rotavaporator (Brinkmann), and dissolved in 5% glucose followed by 20 min sonication as described previously (25, 34). The complex consisting of plasmid DNA expressing human FOXM1 under the control of human CDH5 promoter or empty vector and liposomes was combined at a ratio of 1 μg of DNA to 8 nmol of liposomes. The DNA/liposome complex (50 μg of DNA/mouse) was injected into the retro-orbital venous plexus at 12h post-LPS challenge.

In a separate study, mixture of nanoparticle:plasmid DNA at a ratio of 1 μg DNA to 0.25 mg nanoparticles (15 μg DNA/mouse) was administered to mice of 25 mo. old at 24h post-LPS.

RNAscope in situ hybridization assay and immunostaining: To determine FOXM1 mRNA expression in ECs of COVID-19 patient lungs and control normal donor lungs, a single-plex RNAscope in situ hybridization assay (ACD, Bio-techne, Newark, Calif.) combined with immunofluorescent staining for CD31 as a EC marker was carried out. Briefly, the tissue sections were baked for 1 h at 60° C., deparaffinized, and treated with H₂O₂ for 10 min at room temperature. Target retrieval was performed for 15 min at 100° C., followed by protease treatment for 15 min at 40° C. The sections were then hybridized with human FOXM1 probe (Cat #446941, target region 308-1244 in NM_001243088.1, ACD, Bio-techne) for 2 h at 40° C. followed by signal amplification for 30 min using RNAscope® Multiplex Fluorescent v2 Assay (Cat #333110, ACD, Bio-techne) as per manufacturer's instructions. The signal was developed by incubating the slides with TSA plus Cyanine 5 system (PerkinElmer, Waltham, Mass.) for 30 min. After RNAscope assay, the slides were incubated in blocking buffer (3% BSA, 1% FBS and 0.1% normal donkey serum) for 1 h followed by incubation with a primary antibody against CD31 (Cat #Ab28364, Abcam, Cambridge, Mass.) at 4° C. overnight. The sections were washed and incubated with appropriate anti-rabbit secondary antibody labeled with Alexa Fluor 488 for 1 h. The slides were then counterstained with DAPI and mounted in Prolong Gold Antifade mounting medium (ThermoFisher Scientific).

To quantify FOXM1 expression, a score system of 0-5 was used. 5 represented highest while 1 represented lowest expression in vascular ECs of each vessel. Fifteen 63×fields each section were randomly selected and examined.

Statistical analysis. Statistical significance was determined by one-way ANOVA with a Dunnett post hoc analysis that calculates P values corrected for multiple comparisons using Prism 7 (Graphpad Software, Inc.). Two-group comparisons were analyzed by the unpaired 2-tailed Student's t test for equal variance. Statistical analysis of the survival study was performed with the log-rank (Mantel-Cox) test. P<0.05 denoted the presence of a statistically significant difference. All bars in dot plot figures represent means.

Results

Cells for lung endothelial regeneration originate from resident ECs following polymicrobial sepsis-induced injury.

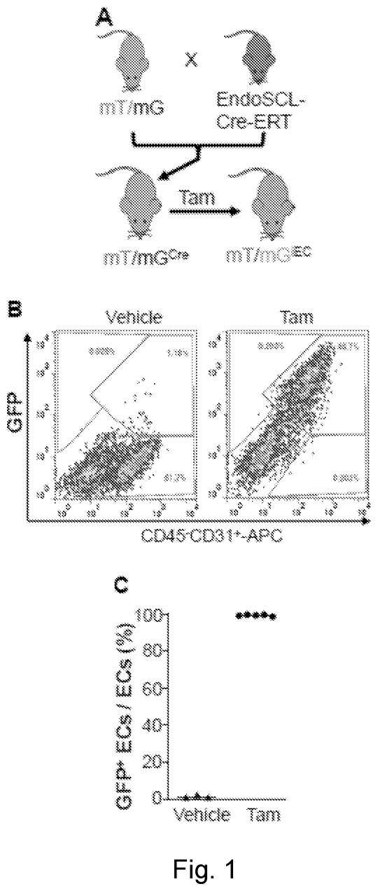

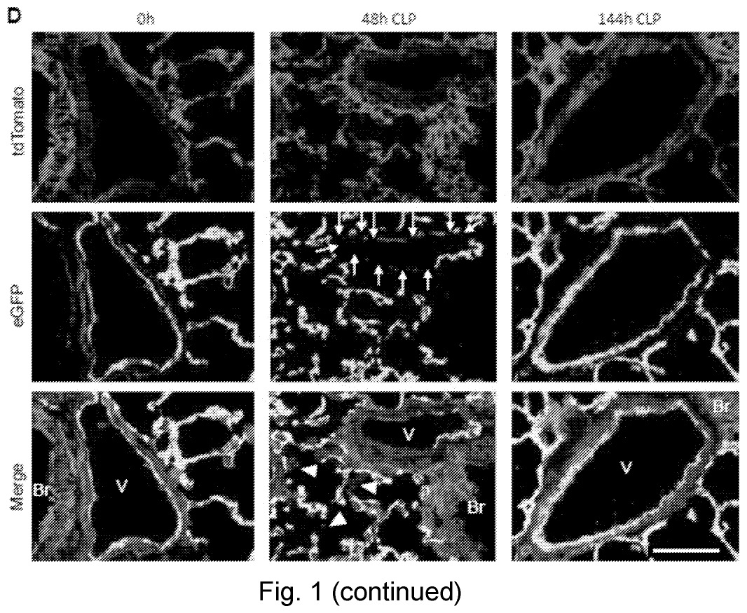

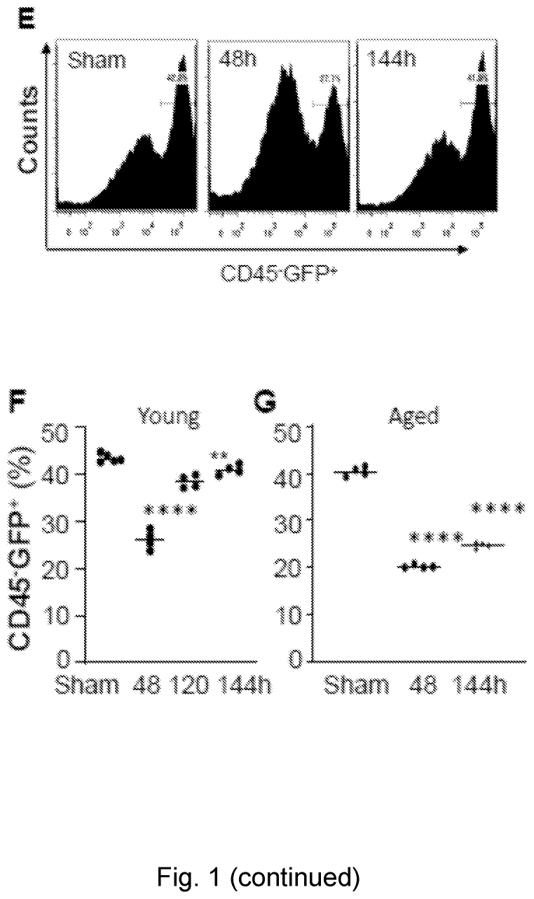

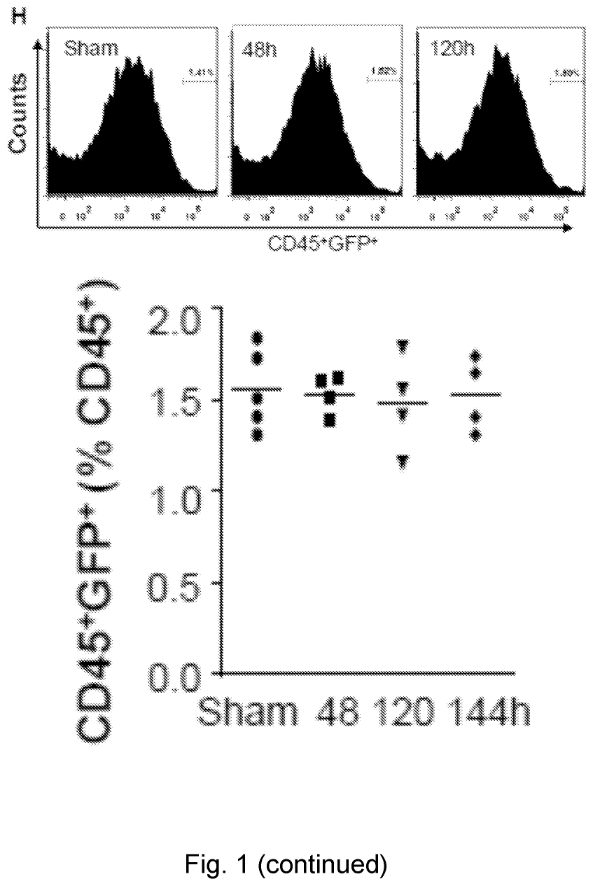

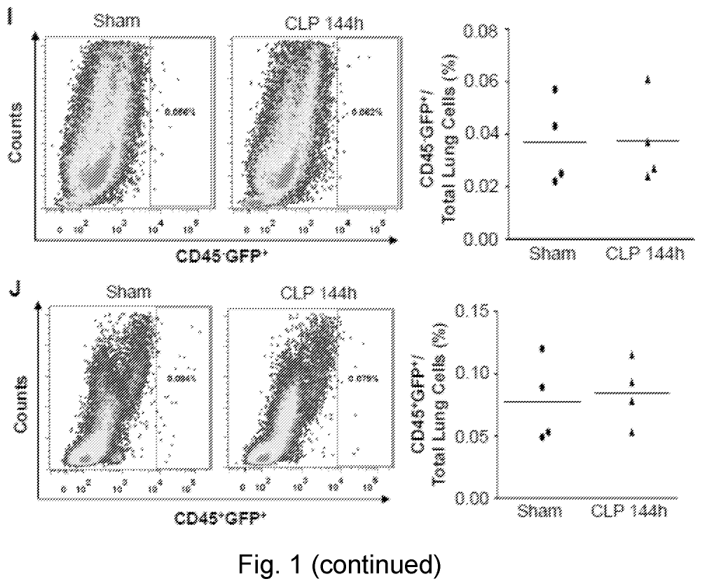

The major pathogenic feature of ALI/ARDS leading to deterioration of vascular barrier function is the precipitous loss of ECs (24, 35-37). To trace the changes of pulmonary ECs following sepsis challenge, we employed a murine tamoxifen-inducible lineage tracing model, mTmG/EndoSCL-CreERT2 mice (FIG. 1A). 95% of lung ECs (CD45⁻CD31⁺) were labeled with green fluorescent protein (GFP) whereas <2% of GFP⁺ cells were either CD45⁺ cells (leukocytes) or CD31− cells (non-ECs) (FIG. 1, B and C). Fluorescence imaging revealed GFP⁺ ECs in capillaries and along the inner surfaces of blood vessels but not bronchioles (FIG. 1D). At 48h post-CLP, which causes lethal peritonitis and polymicrobial sepsis, a well-recognized clinically relevant murine model of sepsis (38, 39), the presence of GFP⁺ ECs was noticeably disrupted along the blood vessel inner surfaces, consistent with loss of ECs seen in patients and animal models; by 144h post-CLP, the blood vessel inner wall was nicely lined with GFP⁺ ECs again. To quantify the changes of pulmonary EC numbers over the course of sepsis-induced injury and recovery, we measured the percentage of CD45-GFP⁺ cells in the whole lung by flow cytometry analysis (FACS) in adult (3-5 mo of age) mice. In sham animals, ˜40% of pulmonary CD45⁻ cells were GFP⁺. At 48h post-CLP, this number had dropped to 25%, but was followed by a steady return to baseline levels by 144h (FIG. 1, E and F). However, the CD45⁺GFP⁺ cell population was remained at steady minimal levels at various times (FIG. 1H), indicating CD45⁺GFP⁺ cells were not involved in endothelial regeneration.

To further determine whether bone marrow-derived cells contribute to post-sepsis endothelial regeneration, we transplanted bone marrow cells from mTmG/EndoSCL-Cre^{ERT2} mice to lethally irradiated WT mice to generate chimeric mice. We observed a small population (<0.1%) of CD45⁻GFP⁺ cells in the chimeric mouse lungs (Sham group). At 144h post-CLP, the percentage of this cell population was unaltered. The CD45⁺GFP⁺ population was also remained steady (FIG. 1, I and J). Thus, bone marrow-derived cells are not attributable to endothelial regeneration. Together, these data demonstrate that lung resident EC is the major cell source for endothelial regeneration in adult mice following inflammatory vascular injury.

Impaired Endothelial Regeneration Leading to Persistent Inflammatory Lung Injury in Aged Mice Following Polymicrobial Sepsis

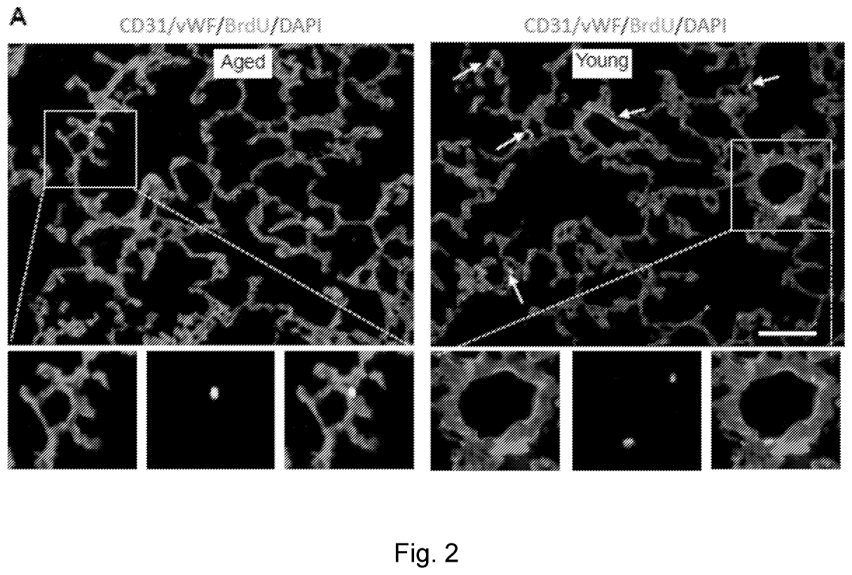

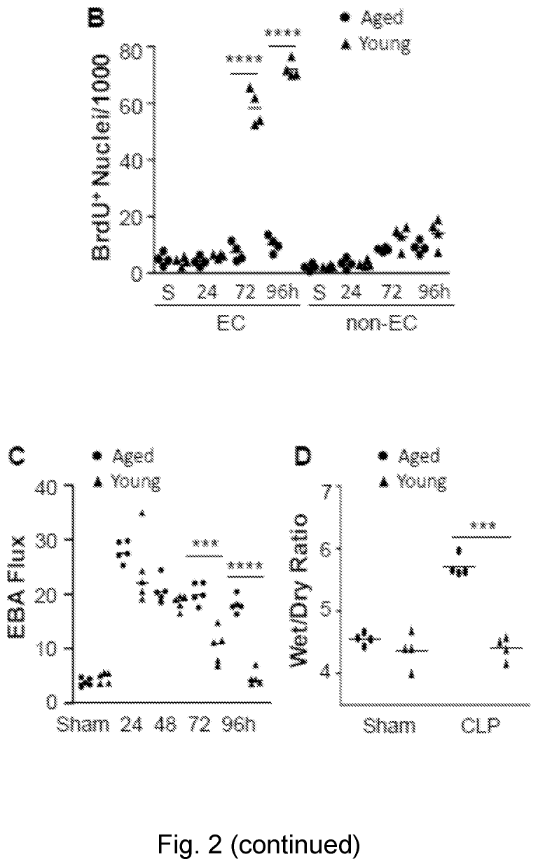

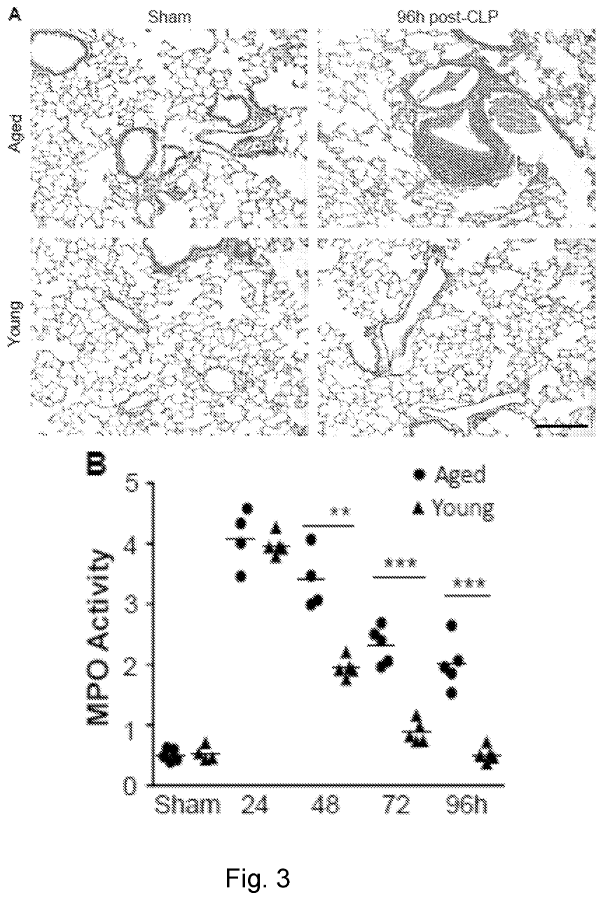

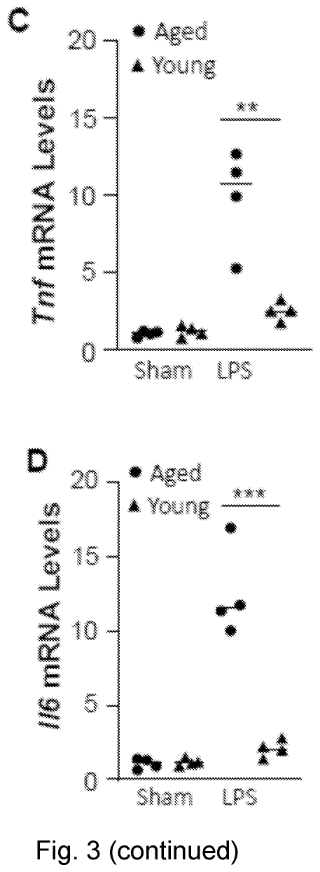

FACS analysis revealed that the lung GFP+ EC population was markedly decreased at 48h post-CLP in aged (19-21 mo) mice as observed in adult mice. However, the GFP+ EC population in aged mice failed to recover and remained low at 144h post-CLP (FIG. 1G). Thus, aging severely impaired the intrinsic endothelial regeneration program following sepsis-induced injury. Anti-BrdU immunostaining, indicative of cell proliferation revealed defective lung endothelial proliferation in aged mice in contrast to young adult mice during the recovery phase (e.g., 72 and 96h post-CLP) (FIG. 2, A and B). Accordingly, EBA assay showed persistent vascular leak indicating impaired vascular repair in the lungs of aged mice in contrast to young adult mice (FIG. 2C). The aged lungs also exhibited marked edema measured by greater lung wet/dry weight ratio at 72h post-CLP (FIG. 2D) and impaired resolution of inflammation during the recovery phase evident by perivascular neutrophil accumulation (FIG. 3A), persistently elevated myeloperoxidase (MPO) activity (FIG. 3B), indicative of neutrophil sequestration, and increased expression of proinflammatory mediators (FIG. 3, C-E).

Aging Impairs Lung Vascular Repair and Resolution of Inflammation Following Endotoxemia

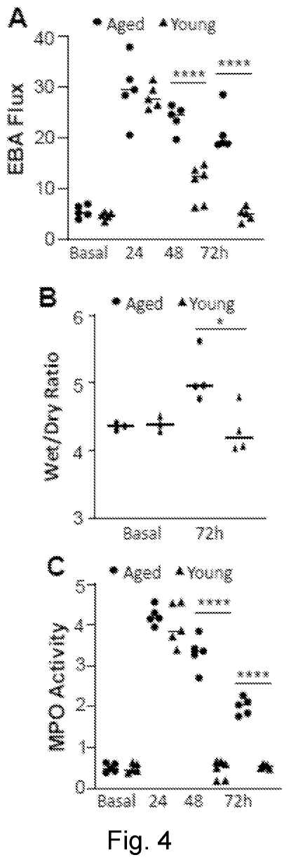

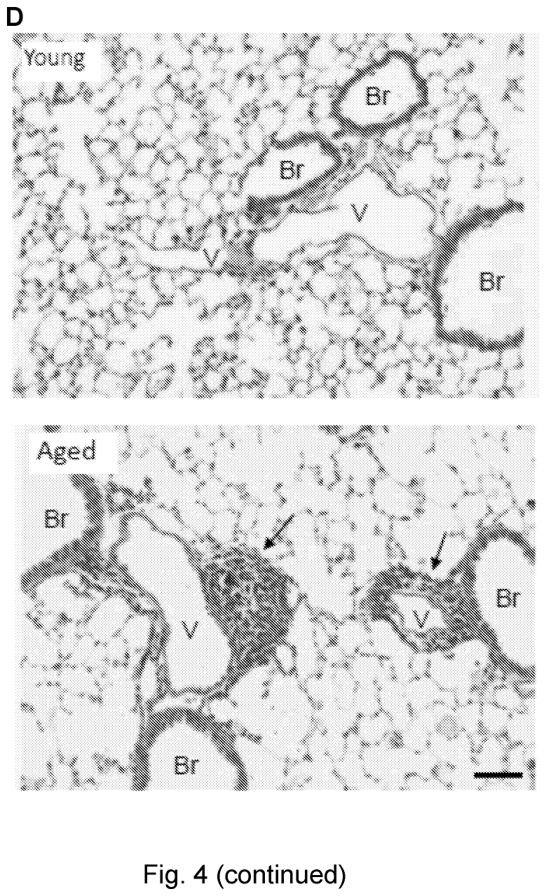

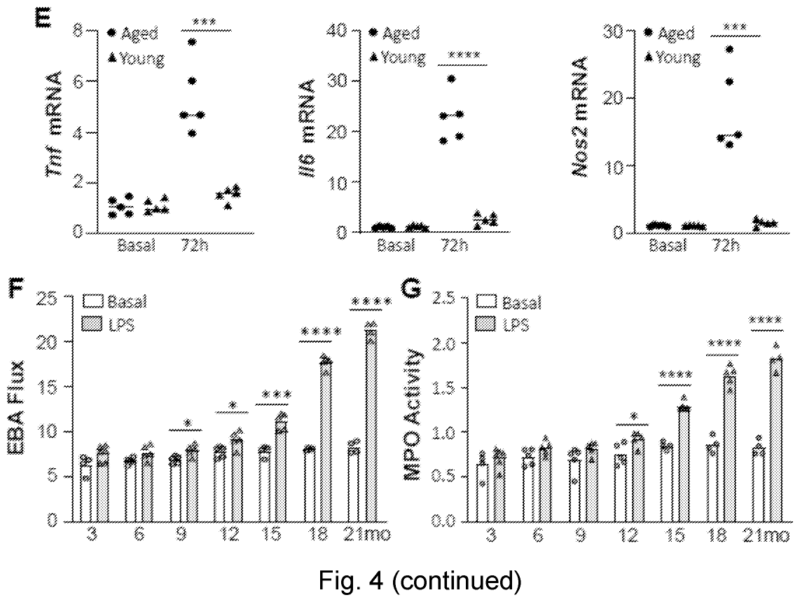

To determine if aged mice also exhibit impaired vascular repair following endotoxemia challenge, aged (19-21 mo) and young (3-5 mo) mice were challenged with LPS. Given that aged mice exhibited greater lung injury indicated by greater EBA flux and MPO activity at 24h post-LPS compared to young adult mice (data not shown), we challenged the aged mice with a lower dose of LPS (e.g., 1.0 mg/kg) to induce similar degree of injury during the injury phase (e.g., 24h) as seen in young adult mice with 2.5 mg/kg of LPS (FIG. 4A). EBA flux in young adult mice was reduced at 48h and returned to basal levels at 72h post-LPS whereas it remained elevated in aged lungs demonstrating defective vascular repair in aged lungs (FIG. 4A). Consistently, aged lungs exhibited edema at 72h post-LPS, which was not observed in young adult mice (FIG. 4B).

MPO activity was also similarly increased at 24h post-LPS in these young adult and aged mice (FIG. 4C). Although MPO activity was returned to basal levels in young adult mice at 72h post-LPS, it remained elevated in aged lungs, indicating neutrophil sequestration, which was consistent with the histological findings by H & E staining showing marked perivascular neutrophil accumulation (FIG. 4D). Furthermore, quantitative RT-PCR analysis demonstrated marked expression of pro-inflammatory genes including TNF, Il6, and Nos2 in aged lungs at 72h post-LPS but not in the lungs of young adult mice (FIG. 4E). Together, these data demonstrated impaired resolution of inflammation in aged lungs following LPS challenge.

To further determine how aging affect vascular repair and inflammation resolution, we challenged the mice at various ages (from 3 to 21 mo old) with LPS and EBA flux and MPO activity were assessed at 72h post-LPS. As shown in FIG. 4F, EBA flux in mice at age of 6 mo or younger were returned to basal levels whereas EBA flux in 9 and 12 mo old mice was not fully recovered but at marginally increased levels however it was markedly elevated in 15 mo old mice and greatly exaggerated in elderly mice, e.g. age of 19 and 21 mo old. We also observed similar changes in MPO activity. Lung MPO activity was not returned to basal levels at 72h post-LPS in mice starting at age of 12 mo and remained marked increased in lungs of mice at age of 15 mo or older, indicating impaired resolution of inflammation (FIG. 4G). Thus, mice at age of 18 mo or older exhibited severely impaired resolution of inflammatory lung injury.

Defective Endothelial Proliferation and Inhibited FoxM1 Induction in Aged Lungs Following LPS Challenge

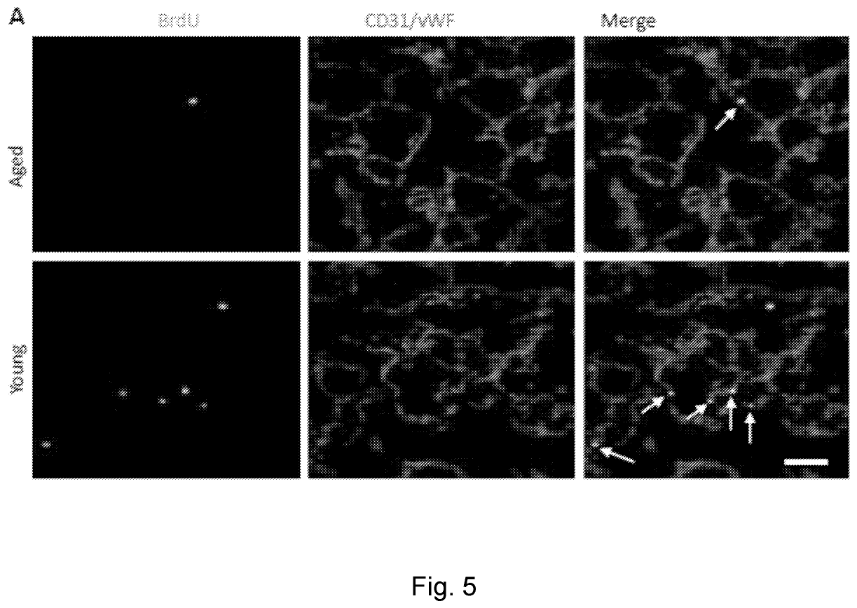

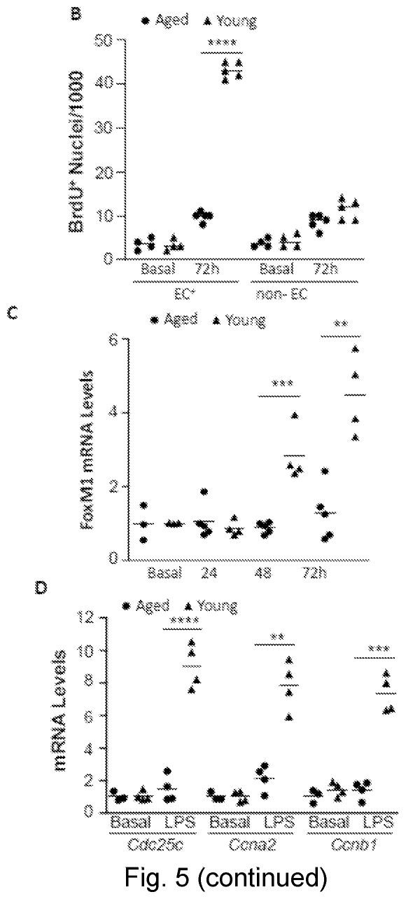

To gain insights into the molecular and cellular mechanisms of impaired vascular repair and inflammation resolution in aged lungs, we first determined lung endothelial proliferation by in vivo BrdU labeling. There was a marked increase of endothelial proliferation in the lungs of young adult mice at 72h post-LPS whereas endothelial proliferation in lungs of aged mice (19-21 mo old) was largely inhibited (FIG. 5, A and B), indicating impaired endothelial regeneration in aged lungs following LPS challenge as seen in aged lungs following polymicrobial sepsis (FIG. 2, A and B). As FoxM1 is a critical reparative transcriptional factor (24, 27, 28), we assessed FoxM1 expression in mouse lungs. FoxM1 was markedly induced in the lungs of young adult mice during the recovery phase but not in aged lungs following LPS challenge (FIG. 5C). Accordingly, FoxM1 target genes essential for cell cycle progression such as Cdc25c, Ccna2 and Ccnb1 were not induced in aged lungs (FIG. 5D).

Normalized Vascular Repair and Inflammation Resolution in Aged FOXM1^{Tg} Mice.

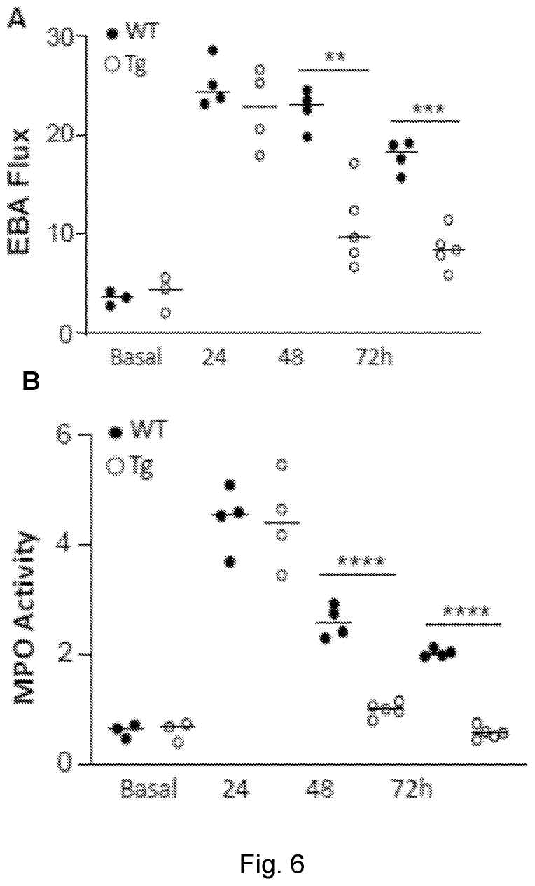

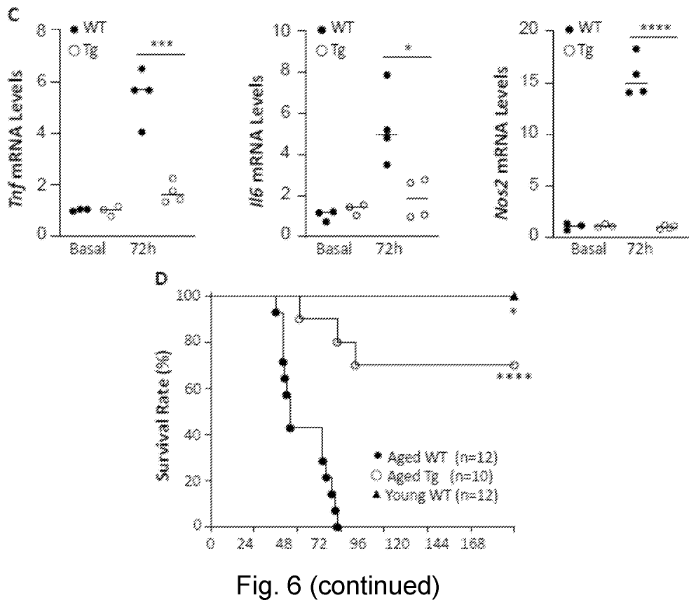

To determine if failure of FoxM1 induction is responsible for the impaired vascular repair and inflammation resolution seen in aged mice, we employed the FOXM1^{Tg} mice expressing human FOXM1 under the control of the −800-base pair Rosa26 promoter (32, 33). EBA flux was similar under basal condition, similarly increased at 24h post-LPS challenge in aged FOXM1^{Tg} mice (19-21 mo old) compared to aged WT mice, demonstrating similar degree of lung vascular injury (FIG. 6A). EBA flux was then reduced at 48h and returned to a level close to basal level at 72h post-LPS in aged FOXM1^{Tg} mice whereas it was persistently elevated in aged WT mice (FIG. 6A). MPO activity was also similarly increased during the injury phase in aged WT and FOXM1^{Tg} mice and reduced during the recovery phase and returned to basal levels at 72h post-LPS in aged FOXM1^{Tg} mice in contrast to aged WT mice (FIG. 6B). Expression of pro-inflammatory genes Tnf, 116, and Nos2 was markedly elevated in aged WT mice but not in aged FOXM1^{Tg} mice (FIG. 6C). These data demonstrate normalized resolution of inflammation in FOXM1^{Tg} mice following LPS challenge.

To determine the survival effect, the mice were challenged with a higher dose of LPS (e.g., 1.5 mg/kg). Aged WT mice exhibited 100% mortality within 3-4 days whereas all young adult mice survived (FIG. 6D). Transgenic expression of FoxM1 also promoted survival of aged mice. 70% of aged FOXM1^{Tg} mice survived in 7 days following LPS challenge.

Therapeutic Expression of FoxM1 Restores Endothelial Regeneration and Resolution of Inflammatory Lung Injury in Aged WT Mice Following LPS Challenge

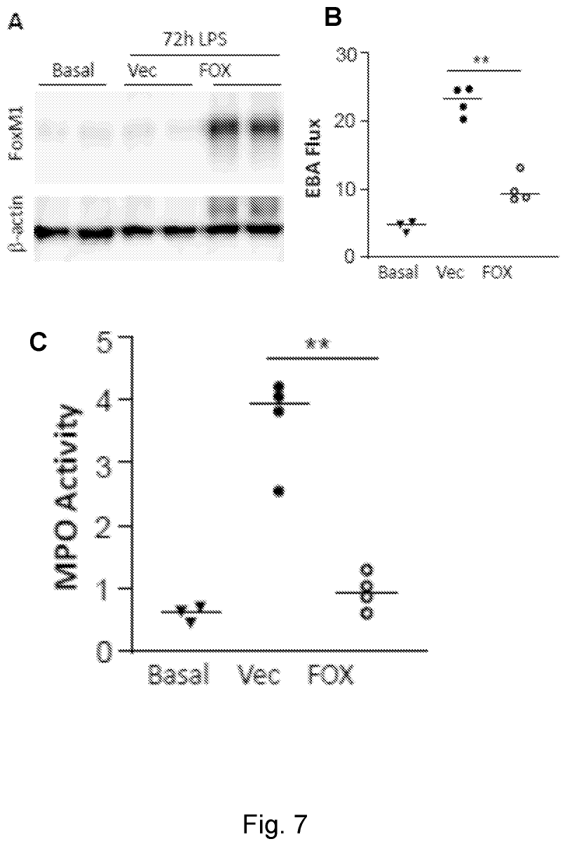

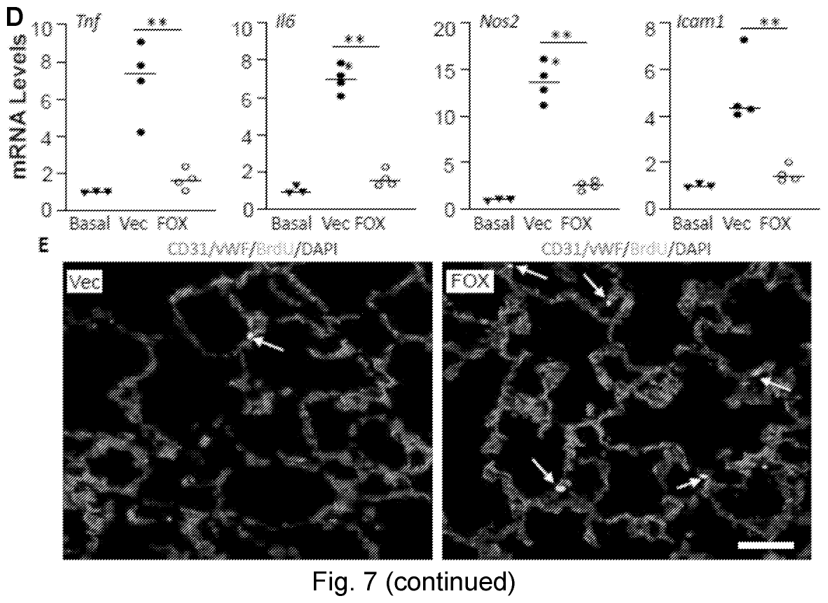

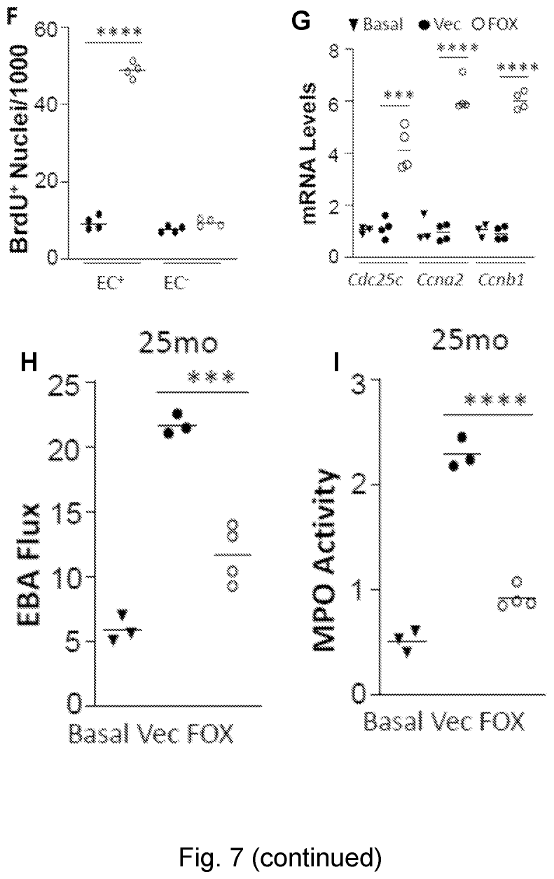

Next, we employed a gene therapy approach to determine if forced FoxM1 expression in lung vascular ECs of aged WT mice can reactivate endothelial proliferation and thus reverse the defective resolution of inflammatory lung injury. A mixture of liposome:plasmid DNA (25, 34) expressing human FOXM1 under the control of human CDH5 promoter (EC-specific) was administered retro-orbitally to 19-20 mo old WT mice at 12h post-LPS challenge (established lung injury). Empty vector DNA was administered to a separate cohort of aged and gender-matched WT mice. As shown in FIG. 7A, liposome transduction of FOXM1 plasmid DNA resulted in a marked increase of FoxM1 expression in aged WT mice at 72h post-LPS compared to vector DNA-transduced mice and untreated control mice (basal). EBA flux was drastically decreased in FOXM1 plasmid DNA-transduced mice compared to vector DNA-transduced mice (FIG. 7B). Lung MPO activity was returned to a level close to basal level in FOXM1 plasmid DNA-transduced mice whereas it was markedly increased in vector DNA-transduced mouse lungs (FIG. 7C). Similarly, expression of pro-inflammatory genes was diminished in lungs of FOXM1 plasmid DNA-transduced mice (FIG. 7D).

We also assessed whether the restored vascular repair and inflammation resolution is attributable to reactivated endothelial proliferation (i.e. regeneration) in aged lungs. BrdU labeling study revealed a marked increase of EC proliferation in lungs of FOXM1 plasmid DNA-transduced mice in sharp contrast to vector DNA-transduced mice (FIG. 7, E and F). Expression of FoxM1 target genes essential for cell cycle progression including Cdc25c, Ccna2, and Ccnb1 was also markedly induced in lungs of FOXM1 plasmid DNA-transduced mice but not in vector DNA-transduced mice (FIG. 7G).

To further determine if forced expression of FoxM1 in mice at very old age (e.g., 25 mo old) can still reactivate the vascular repair program to promote resolution of inflammatory lung injury, we employed our newly developed nanoparticles (which has the potential as a delivery vehicle for gene therapy) to deliver the FOXM1 plasmid DNA to lungs of 25 mo old WT mice. The mixture of nanoparticle:plasmid DNA was administrated retro-orbitally to mice at 24h post-LPS challenge (to ensure the injury response was not affected, i.e. similar degree injury between FOXM1 plasmid DNA- and vector DNA-transduced mice). At 96h post-LPS, lungs were collected for EBA and MPO assessment. As shown in FIG. 7H, lung vascular permeability measured by EBA flux in vector DNA-transduced mice at 96h post-LPS remained markedly elevated whereas it was greatly reduced in FOXM1 plasmid DNA-transduced mice comparable to the observation in 19-21 mo old mice (FIG. 7B). Similarly, lung MPO activity in FOXM1 plasmid DNA-transduced mice was also markedly reduced (FIG. 7I), indicating normalized inflammation resolution.

Failure of FoxM1 Induction in Pulmonary Vascular ECs of Elderly COVID-19 Patients.

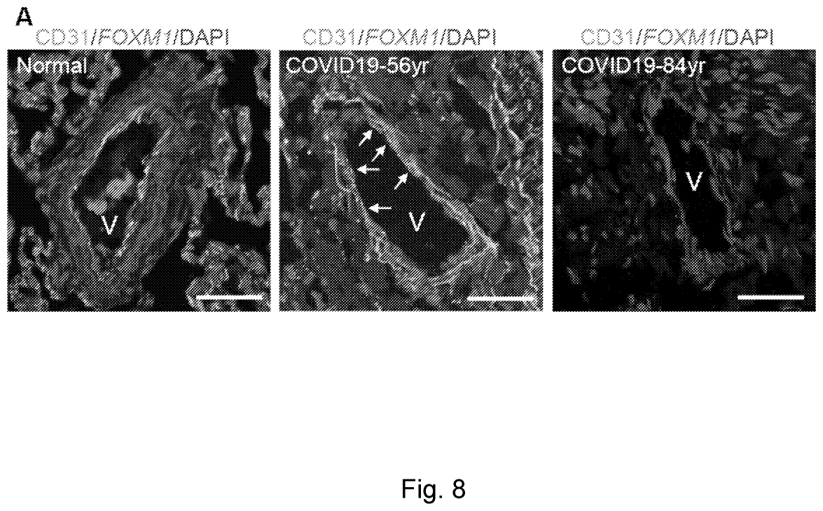

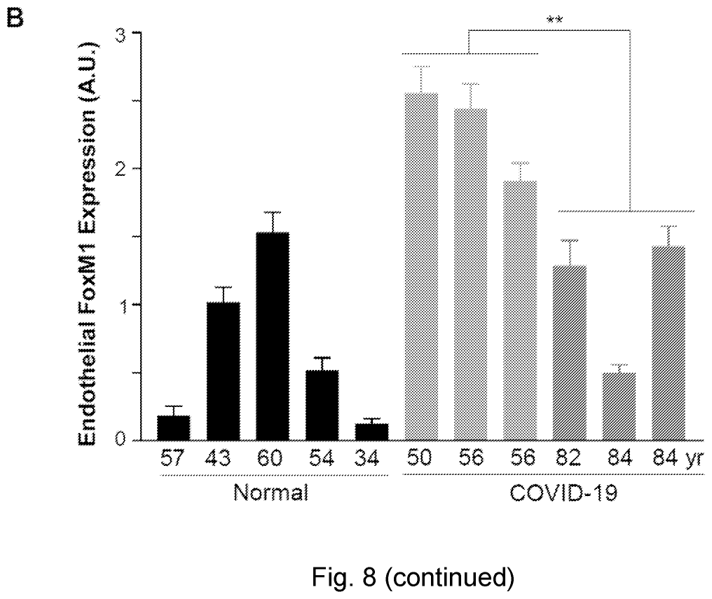

To validate the potential clinical relevance of our findings in aged mice, we collected lung autopsy samples from COVID-19 patients (Table S1) and carried out RNAscope in situ hybridization assay to determine FoxM1 expression. FoxM1 expression in pulmonary vascular ECs was markedly induced in middle-aged COVID-19 patients but not in elderly patients (FIG. 8, A and B). Anti-CD31 immunostaining shows extensive disruption of the endothelial monolayer of COVID-19 patients in both middle-aged and elderly patients (FIG. 8A), manifesting the characteristic feature of endothelial injury of severe COVID-19 patients.

Conclusion

The present study has demonstrated that lung resident EC mediates endothelial regeneration responsible for vascular repair and resulting resolution of inflammation following vascular injury induced by polymicrobial sepsis and aging impairs these processes leading to persistent inflammatory lung injury and high mortality in aged mice. Aging inhibits FoxM1 induction and resulting endothelial proliferation in aged lungs following sepsis challenge. Transgenic expression of FoxM1 normalizes vascular repair and inflammation resolution and promotes survival in aged mice. Therapeutic gene transduction of FoxM1 in lung ECs of aged mice reactivates FoxM1-dependent endothelial regeneration and vascular repair in aged mice. These therapeutic effects were also evident in mice even at age of 25 mos. old. We also observed marked induction of FOXM1 expression in pulmonary vascular ECs of mid-age COVID19 patients but not in elderly patients.

Thus, therapeutic activation of FoxM1 expression by delivery of FOXM1 expressing nucleic acid or pharmacological drugs may represent an effective approach to restore the endothelial barrier integrity and reverse lung edema in the prevention and treatment of COVID-19 and COVID-19 respiratory distress and multi-organ failure, sepsis and ARDS as well as vascular diseases with diminished FOXM1 expression including but not limited to restenosis and critical limb ischemia in elderly patients and adult patients.

### REFERENCES FOR EXAMPLE 1

1. Rubenfeld G D, Caldwell E, Peabody E, Weaver J, Martin D P, Neff M, Stern E J, Hudson L D. Incidence and outcomes of acute lung injury. N Engl J Med 2005; 353: 1685-1693.
2. Matthay M A, Ware L B, Zimmerman G A. The acute respiratory distress syndrome. J Clin Invest 2012; 122: 2731-2740.
3. Matthay M A, Zemans R L, Zimmerman G A, Arabi Y M, Beitler J R, Mercat A, Herridge M, Randolph A G, Calfee C S. Acute respiratory distress syndrome. Nat Rev Dis Primers 2019; 5: 18.
4. Goldenberg N M, Steinberg B E, Slutsky A S, Lee W L. Broken barriers: a new take on sepsis pathogenesis. Sci Transl Med 2011; 3: 88ps25.
5. Lee W L, Slutsky A S. Sepsis and endothelial permeability. N Engl J Med 2010; 363: 689-691.
6. Minamino T, Komuro I. Regeneration of the endothelium as a novel therapeutic strategy for acute lung injury. J Clin Invest 2006; 116: 2316-2319.
7. Aird W C. The role of the endothelium in severe sepsis and multiple organ dysfunction syndrome. Blood 2003; 101: 3765-3777.
8. Angus D C, Linde-Zwirble W T, Lidicker J, Clermont G, Carcillo J, Pinsky M R. Epidemiology of severe sepsis in the United States: analysis of incidence, outcome, and associated costs of care. Crit Care Med 2001; 29: 1303-1310.
9. Mirzanejad Y, Roman S, Talbot J, Nicolle L. Pneumococcal bacteremia in two tertiary care hospitals in Winnipeg, Canada. Pneumococcal Bacteremia Study Group. Chest 1996; 109: 173-178.
10. Gee M H, Gottlieb J E, Albertine K H, Kubis J M, Peters S P, Fish J E. Physiology of aging related to outcome in the adult respiratory distress syndrome. J Appl Physiol (1985) 1990; 69: 822-829.
11. Sloane P J, Gee M H, Gottlieb J E, Albertine K H, Peters S P, Burns J R, Machiedo G, Fish J E. A multicenter registry of patients with acute respiratory distress syndrome. Physiology and outcome. Am Rev Respir Dis 1992; 146: 419-426.
12. Griffith D, Idell S. Approach to adult respiratory distress syndrome and respiratory failure in elderly patients. Clin Chest Med 1993; 14: 571-582.
13. Kaestner K H, Knochel W, Martinez D E. Unified nomenclature for the winged helix/forkhead transcription factors. Genes Dev 2000; 14: 142-146.
14. Clark K L, Halay E D, Lai E, Burley S K. Co-crystal structure of the HNF-3/fork head DNA-recognition motif resembles histone H5. Nature 1993; 364: 412-420.
15. Korver W, Roose J, Clevers H. The winged-helix transcription factor Trident is expressed in cycling cells. Nucleic Acids Res 1997; 25: 1715-1719.
16. Ye H, Kelly T F, Samadani U, Lim L, Rubio S, Overdier D G, Roebuck K A, Costa R H. Hepatocyte nuclear factor 3/fork head homolog 11 is expressed in proliferating epithelial and mesenchymal cells of embryonic and adult tissues. Mol Cell Biol 1997; 17: 1626-1641.
17. Yao K M, Sha M, Lu Z, Wong G G. Molecular analysis of a novel winged helix protein, WIN. Expression pattern, DNA binding property, and alternative splicing within the DNA binding domain. J Biol Chem 1997; 272: 19827-19836.
18. Wang I C, Chen Y J, Hughes D, Petrovic V, Major M L, Park H J, Tan Y, Ackerson T, Costa R H. Forkhead box M1 regulates the transcriptional network of genes essential for mitotic progression and genes encoding the SCF (Skp2-Cks1) ubiquitin ligase. Mol Cell Biol 2005; 25: 10875-10894.
19. Laoukili J, Kooistra M R, Bras A, Kauw J, Kerkhoven R M, Morrison A, Clevers H, Medema R H. FoxM1 is required for execution of the mitotic programme and chromosome stability. Nat Cell Biol 2005; 7: 126-136.
20. Bolte C, Zhang Y, Wang I C, Kalin T V, Molkentin J D, Kalinichenko V V. Expression of Foxm1 transcription factor in cardiomyocytes is required for myocardial development. PLoS One 2011; 6: e22217.
21. Ustiyan V, Wang I C, Ren X, Zhang Y, Snyder J, Xu Y, Wert S E, Lessard J L, Kalin T V, Kalinichenko V V. Forkhead box M1 transcriptional factor is required for smooth muscle cells during embryonic development of blood vessels and esophagus. Dev Biol 2009; 336: 266-279.
22. Kalin T V, Wang I C, Meliton L, Zhang Y, Wert S E, Ren X, Snyder J, Bell S M, Graf L, Jr., Whitsett J A, Kalinichenko V V. Forkhead Box m1 transcription factor is required for perinatal lung function. Proc Natl Acad Sci USA 2008; 105: 19330-19335.
23. Hou Y, Li W, Sheng Y, Li L, Huang Y, Zhang Z, Zhu T, Peace D, Quigley J G, Wu W, Zhao Y Y, Qian Z. The transcription factor Foxm1 is essential for the quiescence and maintenance of hematopoietic stem cells. Nat Immunol 2015; 16: 810-818.
24. Zhao Y Y, Gao X P, Zhao Y D, Mirza M K, Frey R S, Kalinichenko V V, Wang I C, Costa R H, Malik A B. Endothelial cell-restricted disruption of FoxM1 impairs endothelial repair following LPS-induced vascular injury. J Clin Invest 2006; 116: 2333-2343.
25. Mirza M K, Sun Y, Zhao Y D, Potula H H, Frey R S, Vogel S M, Malik A B, Zhao Y Y. FoxM1 regulates re-annealing of endothelial adherens junctions through transcriptional control of beta-catenin expression. J Exp Med 2010; 207: 1675-1685.
26. Zhao Y D, Huang X, Yi F, Dai Z, Qian Z, Tiruppathi C, Tran K, Zhao Y Y. Endothelial FoxM1 mediates bone marrow progenitor cell-induced vascular repair and resolution of inflammation following inflammatory lung injury. Stem Cells 2014; 32: 1855-1864.
27. Liu Y, Sadikot R T, Adami G R, Kalinichenko V V, Pendyala S, Natarajan V, Zhao Y Y, Malik A B. FoxM1 mediates the progenitor function of type II epithelial cells in repairing alveolar injury induced by Pseudomonas aeruginosa. J Exp Med 2011; 208: 1473-1484.
28. Wang X, Kiyokawa H, Dennewitz M B, Costa R H. The Forkhead Box m1b transcription factor is essential for hepatocyte DNA replication and mitosis during mouse liver regeneration. Proc Natl Acad Sci USA 2002; 99: 16881-16886.
29. Gothert J R, Gustin S E, van Eekelen J A, Schmidt U, Hall M A, Jane S M, Green A R, Gottgens B, Izon D J, Begley C G. Genetically tagging endothelial cells in vivo: bone marrow-derived cells do not contribute to tumor endothelium. Blood 2004; 104: 1769-1777.
30. Nussbaum C, Bannenberg S, Keul P, Graler M H, Goncalves-de-Albuquerque C F, Korhonen H, von Wnuck Lipinski K, Heusch G, de Castro Faria Neto H C, Rohwedder I, Gothert J R, Prasad V P, Haufe G, Lange-Sperandio B, Offermanns S, Sperandio M, Levkau B. Sphingosine-1-phosphate receptor 3 promotes leukocyte rolling by mobilizing endothelial P-selectin. Nat Commun 2015; 6: 6416.
31. Tran K A, Zhang X, Predescu D, Huang X, Machado R F, Gothert J R, Malik A B, Valyi-Nagy T, Zhao Y Y. Endothelial beta-Catenin Signaling Is Required for Maintaining Adult Blood-Brain Barrier Integrity and Central Nervous System Homeostasis. Circulation 2016; 133: 177-186.
32. Kalinichenko V V, Gusarova G A, Tan Y, Wang I C, Major M L, Wang X, Yoder H M, Costa R H. Ubiquitous expression of the forkhead box M1B transgene accelerates proliferation of distinct pulmonary cell types following lung injury. J Biol Chem 2003; 278: 37888-37894.
33. Huang X, Zhao Y Y. Transgenic expression of FoxM1 promotes endothelial repair following lung injury induced by polymicrobial sepsis in mice. PLoS One 2012; 7: e50094.
34. Huang X, Dai Z, Cai L, Sun K, Cho J, Albertine K H, Malik A B, Schraufnagel D E, Zhao Y Y. Endothelial p110gammaPI3K Mediates Endothelial Regeneration and Vascular Repair After Inflammatory Vascular Injury. Circulation 2016; 133: 1093-1103.
35. Fujita M, Kuwano K, Kunitake R, Hagimoto N, Miyazaki H, Kaneko Y, Kawasaki M, Maeyama T, Hara N. Endothelial cell apoptosis in lipopolysaccharide-induced lung injury in mice. Int Arch Allergy Immunol 1998; 117: 202-208.
36. Kitamura Y, Hashimoto S, Mizuta N, Kobayashi A, Kooguchi K, Fujiwara I, Nakajima H. Fas/FasL-dependent apoptosis of alveolar cells after lipopolysaccharide-induced lung injury in mice. Am J Respir Crit Care Med 2001; 163: 762-769.
37. Gill S E, Rohan M, Mehta S. Role of pulmonary microvascular endothelial cell apoptosis in murine sepsis-induced lung injury in vivo. Respir Res 2015; 16: 109.
38. Buras J A, Holzmann B, and Sitkovsky M. Animal models of sepsis: setting the stage. Nat Rev Drug Discov 2005; 4: 854-865.
39. Rittirsch D, Huber-Lang M S, Flierl M A, and Ward P A. Immunodesign of experimental sepsis by cecal ligation and puncture. Nat Protoc 2009; 4: 31-36.

Example 2. Repurposing rabeprazole or phenazopyridine as monotherapy or combination therapy with NAC, or Dexamethasone or NOX2 inhibitors or other drug(s) for the treatment of COVID-19, COVID-19 sepsis, COVID-19 respiratory distress and multi-organ failure, sepsis and ARDS as well as vascular diseases with impaired HIF-1a signaling and/or diminished FOXM1 expression including but not limited to restenosis and critical limb ischemia in elderly patients and adult patients.

Hypoxia-inducible factors (HIFs) comprised of an O₂-sensitive α-subunit (mainly HIF-1α and HIF-2α) and a constitutively expressed β-subunit are key transcription factors mediating adaptive responses to hypoxia and ischemia (1,2). Our study shows that hypoxia-inducible factor (HIF)-1α is required for endothelial regeneration and vascular repair and thus resolution of inflammatory lung injury in (young) adult mice (3). FoxM1 expression was not induced in lung vascular ECs in Hif1α EC-specific knockout mice and restoration of FoxM1 in ECs normalized vascular repair and resolution of inflammation in Hif1α EC-specific knockout mice. To identify FDA-approved drug(s) that could activate HIF-1α/FoxM1 signaling and thereby provide novel therapeutic agents for treatment of COVID-19 respiratory distress and multi-organ failure, sepsis and ARDS and vascular diseases including but not limited to restenosis and critical limb ischemia in elderly patients and adult patients, we carried out high-throughput screening of the Prestwick Chemical Library of FDA-approved drugs (1280 compounds) employing stable cell line containing the HIF-response element. Rabeprazole and Phenazopyridine were the 2 activators identified by the inventor, which can induce FoxM1 expression and promote vascular repair and resolution of inflammation in aged mice and also young adult mice following sepsis challenge.

Rabeprazole is a HIF1α activator which can reactivate FoxM1 expression and vascular repair in aged lungs.

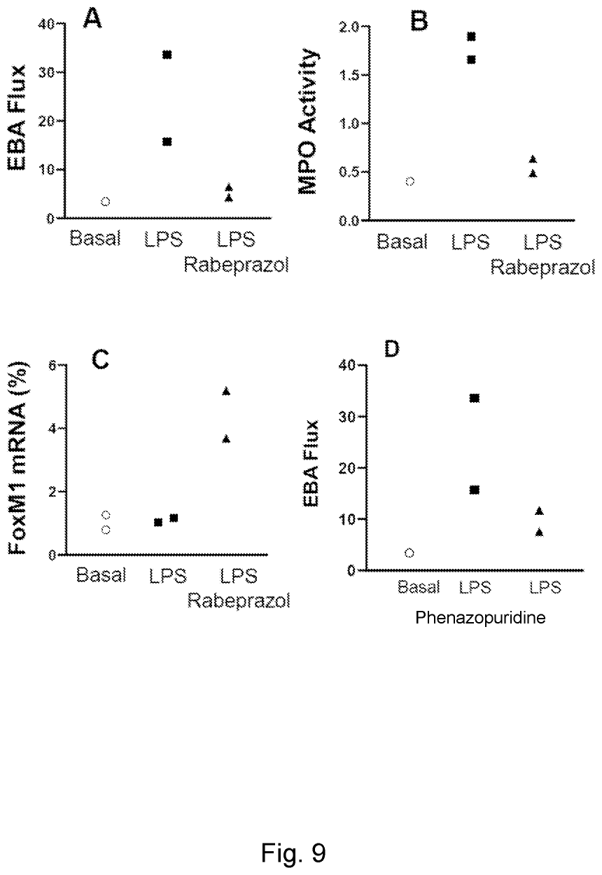

To test if Rabeprazole can activate vascular repair and resolution of inflammation in aged mice, we challenged aged mice (22 mo. old) with LPS to induce endotoxemia and inflammatory injury, and then treated with Rabeprazole at 6h and 24h post-LPS. Lungs were collected for Evan blue-conjugated albumin (EBA) assay (a measurement of vascular permeability) and myeloperoxidase (MPO) activity assay at 72h post-LPS. As shown in FIG. 9A, rabeprazole treatment resulted in a recovery of vascular permeability whereas untreated mice exhibited persistent increase of EBA, i.e., vascular injury. MPO activity in rabeprazole-treated mice was also returned to levels seen in basal mice (FIG. 9B). Furthermore, we determined if rabeprazole can activate FoxM1 expression in the lung in aged mice following sepsis challenge. We observed a 3-6-fold increase of FoxM1 expression compared to either basal or LPS-treated mice (FIG. 9C).

Rabeprazole can also facilitate vascular repair in young adult mice.

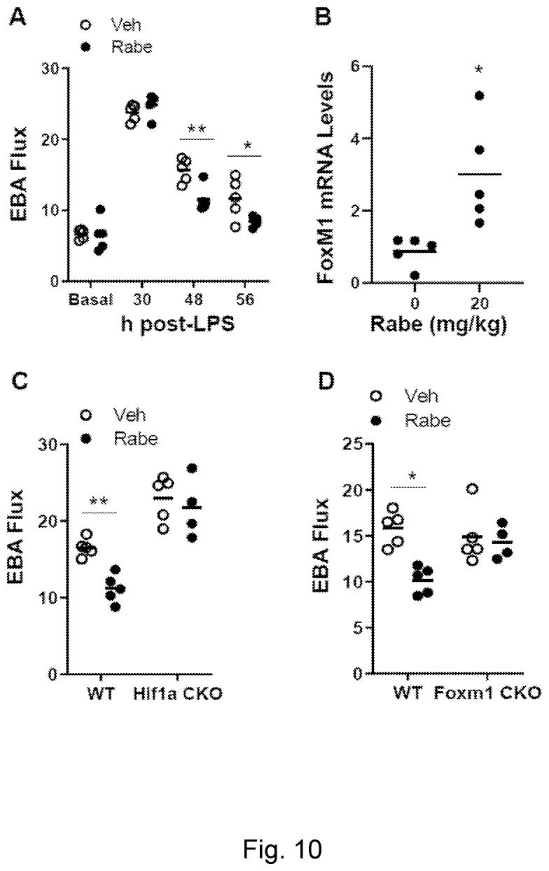

To test if Rabeprazole can facilitate vascular repair in young adult mice, we challenged 3-5 mos. old mice with LPS to induce endotoxemia and inflammatory injury, and then treated with Rabeprazole (18 mg/kg, oral) at 6h and 24h post-LPS. Lungs were collected for EBA) assay at various times post-LPS. As shown in FIG. 10A, Rabeprazol treatment resulted in a quick recovery of vascular permeability at 56h post-LPS whereas had no effect during peak injury at 30h post-LPS. Furthermore, we determined if Rabeprazole can activate FoxM1 expression in the lung following sepsis challenge (FIG. 10B). We observed a 3-fold increase of FoxM1 expression compared to either basal or LPS-treated mice (FIG. 10B).

Rabeprazole promote HIF-1a/FoxM1-dependent vascular repair.

To determine if Rabeprazole-induced vascular repair is through activation of HIF-1a, WT and Hif1a EC-specific knockout mice (4 mos. old) were challenged with LPS and then treated with Rabeprazole at 6 h and 24h post-LPS. At 52h post-LPS, lungs were collected EBA assay. As shown in FIG. 10C, Rabeprazole-induced vascular repair seen in WT mice was inhibited in Hif1a KO mice.

we also determine if Rabeprazole-induced vascular repair is mediated by endothelial FoxM1. WT and Foxm1 EC-specific knockout mice (3-5 mos. old) were challenged with LPS and then treated with Rabeprazole at 6 h and 24h post-LPS. At 52h post-LPS, lungs were collected EBA assay. Rabeprazole-induced vascular repair seen in WT mice was also inhibited in Foxm1 EC KO mice (FIG. 10D).

Together, these data demonstrate that Rabeprazole can efficiently activate FoxM1-dependent endothelial regeneration, vascular repair and resolution of inflammation in aged mice as well as young adult mice. Thus, Rabeprazole and its analogues can be repurposed for treatment of elderly patients and also adult patients with COVID-19, COVID-19 sepsis, COVID-19 respiratory distress and multi-organ failure, sepsis, ARDS, and multi-organ failure to reduce morbidity and mortality as a monotherapy or combination therapy with either Dexamethasone, NAC, NOX2 inhibitors (Apocynin, Ebselen, APX-115, Thienopyridine, NOX2ds-tat, NOX2 inhibiting nucleic acids), Phenazopyridine or its analogues.

Additionally, as rabeprazole can activate endothelial regeneration and vascular repair, it can also be repurposed for treatment of vascular diseases with impaired HIF-1a signaling or diminished FOXM1 expression including restenosis, and critical limb ischemia (to promote angiogenesis).

Besides rabeprazole, we also found another drug, phenazopyridine, which could also promote vascular repair in aged mice (FIG. 9D). Thus, phenazopyridine and its analogues can also be repurposed for treatment of elderly patients and also adult patients with COVID-19, COVID-19 sepsis, COVID-19 respiratory distress and multi-organ failure, sepsis, ARDS to reduce morbidity and mortality as a monotherapy or combination therapy with either Dexamethasone, RV, NAC, NOX2 inhibitors (Apocynin, Ebselen, APX-115, Thienopyridine, NOX2ds-tat, NOX2 inhibiting nucleic acids). Phenazopyridine can also be repurposed for treatment of cardiovascular diseases including restenosis, and critical limb ischemia, and anemia.

Combination of rabeprazole or its analogue with Phenazopyridine or its analogue can be repurposed for treatment of vascular diseases associated with impaired HIF-1a signaling and/or diminished FOXM1 expression including but not limited to restenosis, and critical limb ischemia.

### REFERENCE FOR EXAMPLE 2

1. Majmundar A J, Wong W J, Simon M C. Hypoxia-inducible factors and the response to hypoxic stress. Mol Cell. 2010; 40: 294-309.
2. Semenza G L. Hypoxia-inducible factors in physiology and medicine. Cell. 2012; 148: 399-408.
3. Huang X, Zhang X, Zhao D X, Yin J, Hu G, Evans C E, Zhao Y Y. Endothelial Hypoxia-inducible Factor-1α is Required for Vascular Repair and Resolution of Inflammatory Lung injury through Forkhead Box Protein M1. Am J Pathol. 2019 August; 189(8):1664-1679.

Example 3: EGLN1 deficiency normalizes vascular repair and reactivates FoxM1 expression in lungs of aged mice. EGLN1 inhibitors (e.g., roxadustat, molidustat, vadadustat, and desidustat) and Egln1 inhibiting nucleic acid as a monotherapy or combination therapy with one or more of Dexamethasone, RV, NAC, NOX2 inhibitors (Apocynin, Ebselen, APX-115, Thienopyridine, NOX2ds-tat, NOX2 inhibiting nucleic acid) for treatment of COVID-19, COVID-19 sepsis, COVID-19 respiratory distress and multi-organ failure, sepsis, ARDS, and multi-organ failure. And also for treatment of vascular diseases associated with impaired HIF-1a signaling and/or diminished FOXM1 expression but not limited to including restenosis and critical limb ischemia.

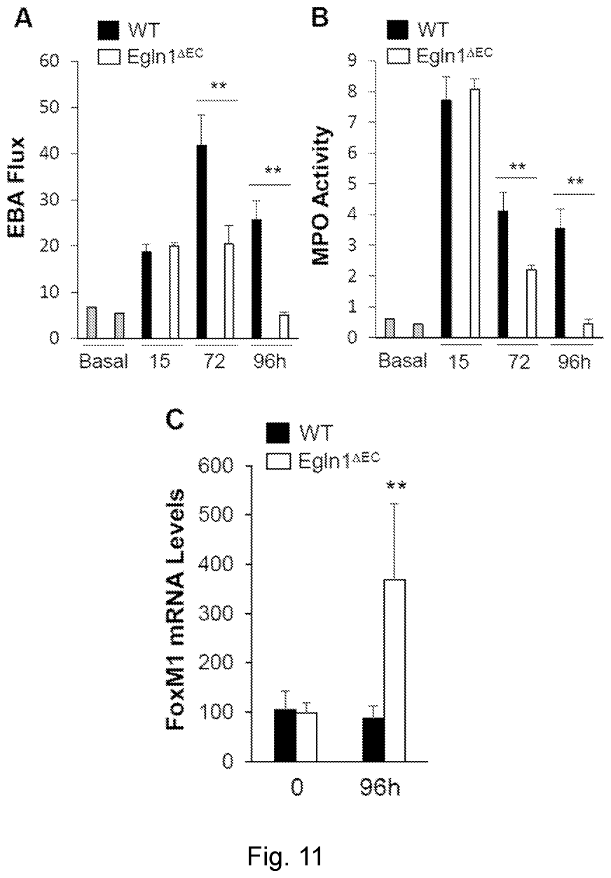

As O₂ sensors, HIF prolyl-4 hydroxylases [prolyl hydroxylase domain-containing enzymes (PHDs), also known as EGLN1-3] use molecular O₂ as a substrate to hydroxylate specific proline residues of HIF-α. Hydroxylation promotes HIF-α binding to the von Hippel-Lindau (VHL) ubiquitin E3 ligase resulting in ubiquitination and subsequent degradation by proteasome (1-4). EGLN1 (i.e. PHD2) is responsible for the majority of HIF-α hydroxylation while EGLN2 and EGLN3 play compensatory roles under certain conditions (5-8). To determine the role of EGLN1 in regulating FoxM1 expression and vascular repair in aged mice, WT and Egln1^{ΔEC} mice with EC-restricted disruption of Egln1 at age of 21 months were challenged with LPS. We observed similar degree of vascular injury and lung MPO activity in WT and Egln1^{ΔEC} mice at 15h post-LPS challenge (FIG. 11). WT mice exhibited persistent inflammatory lung injury whereas vascular permeability and MPO activity were returned to basal levels in Egln1^{ΔEC} mice at 96h post-LPS. Consistently, FoxM1 was markedly induced in Egln1^{ΔEC} lungs at 96h post-LPS in contrast to WT lungs (FIG. 11). Thus, EGLN1 inhibitors (e.g., roxadustat, molidustat, vadadustat, and desidustat) and Egln1 inhibiting nucleic acid including, but not limited to antisense oligo, siRNA, shRNA, guide RNA are novel therapeutic agents to re-activate FoxM1-dependent vascular repair in aged subjects for treatment of COVID-19, COVID-19 sepsis, COVID-19 respiratory distress and multi-organ failure, sepsis, ARDS, and multi-organ failure.

### REFERENCE FOR EXAMPLE 3

1. Ivan M, Kondo K, Yang H, Kim W, Valiando J, Ohh M, Salic A, Asara J M, Lane W S, Kaelin W G, Jr. HIFalpha targeted for VHL-mediated destruction by proline hydroxylation: implications for O2 sensing. Science. 2001; 292: 464-468.
2. Epstein A C, Gleadle J M, McNeill L A, Hewitson K S, O'Rourke J, Mole D R, Mukherji M, Metzen E, Wilson M I, Dhanda A, Tian Y M, Masson N, Hamilton D L, Jaakkola P, Barstead R, Hodgkin J, Maxwell P H, Pugh C W, Schofield C J, Ratcliffe P J. C. elegans EGL-9 and mammalian homologs define a family of dioxygenases that regulate HIF by prolyl hydroxylation. Cell. 2001; 107: 43-54.
3. Jaakkola P, Mole D R, Tian Y M, Wilson M I, Gielbert J, Gaskell S J, von Kriegsheim A, Hebestreit H F, Mukherji M, Schofield C J, Maxwell P H, Pugh C W, Ratcliffe P J. Targeting of HIF-alpha to the von Hippel-Lindau ubiquitylation complex by O2-regulated prolyl hydroxylation. Science. 2001; 292: 468-472.
4. Bishop T, Ratcliffe P J. HIF hydroxylase pathways in cardiovascular physiology and medicine. Circ Res. 2015; 117: 65-79.
5. Berra E, Benizri E, Ginouves A, Volmat V, Roux D, Pouyssegur J. HIF prolyl-hydroxylase 2 is the key oxygen sensor setting low steady-state levels of HIF-1alpha in normoxia. EMBO J. 2003; 22: 4082-4090.
6. Appelhoff R J, Tian Y M, Raval R R, Turley H, Harris A L, Pugh C W, Ratcliffe P J, Gleadle J M. Differential function of the prolyl hydroxylases PHD1, PHD2, and PHD3 in the regulation of hypoxia-inducible factor. J Biol Chem. 2004; 279: 38458-38465.
7. Minamishima Y A, Moslehi J, Bardeesy N, Cullen D, Bronson R T, Kaelin W G, Jr. Somatic inactivation of the PHD2 prolyl hydroxylase causes polycythemia and congestive heart failure. Blood. 2008; 111: 3236-3244.
8. Takeda K, Cowan A, Fong G H. Essential role for prolyl hydroxylase domain protein 2 in oxygen homeostasis of the adult vascular system. Circulation. 2007; 116: 774-781.

Example 4: Dimethyloxalylglycine (DMOG) analogues including roxadustat, molidustat, vadadustat, and desidustat as a monotherapy or combination therapy with one or more of Dexamethasone, RV, NAC, NOX2 inhibitors (Apocynin, Ebselen, APX-115, Thienopyridine, NOX2ds-tat, NOX2 inhibiting nucleic acid) to treat COVID-19, COVID-19 sepsis, COVID-19 respiratory distress and multi-organ failure, sepsis, ARDS, and multi-organ failure in elderly patients and adult patients, and also for treatment of vascular diseases associated with impaired HIF-1a signaling and/or diminished FOXM1 expression but not limited to including restenosis and critical limb ischemia.

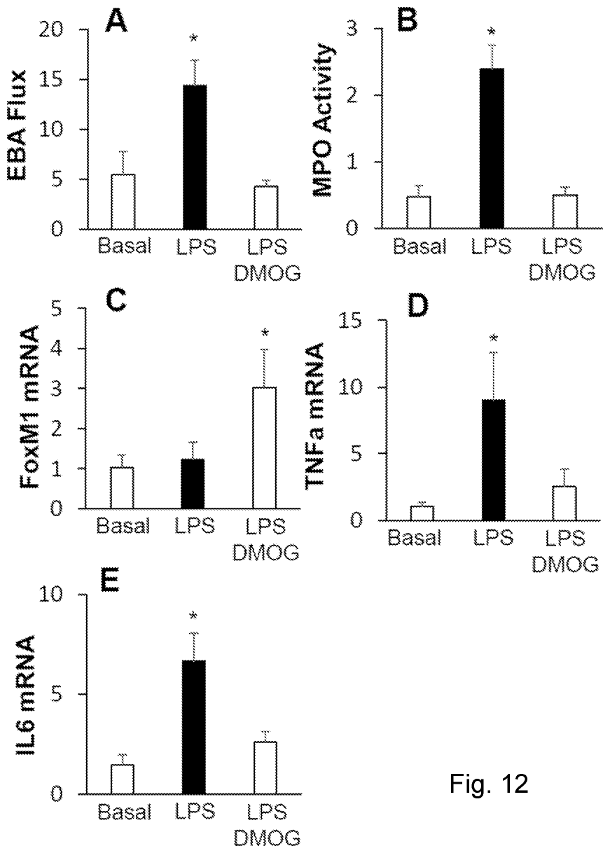

DMOG is a cell permeable EGLN/PHD inhibitor which stabilizes HIF-α. We next determined whether DMOG can activate the vascular repair program in aged mice. Aged mice (21 mo. old) were challenged with LPS and then treated with DMOG at 12 or 24h post-LPS and lung tissues were collected at 72h post-LPS. As shown in FIG. 12A, vascular permeability (EBA flux) in DMOG-treated mice was returned to the levels seen in basal mice whereas LPS-challenged mice without DMOG treatment exhibited persistent vascular injury at 72h post-LPS. Consistently, MPO activity was also returned to basal levels in DMOG-treated mice in sharp contrast to untreated mice at 72h post-LPS. (FIG. 12B). Quantitative RT-PCR analysis demonstrate a 3-fold increase of FoxM1 expression in the lungs of DMOG treated mice compared to either basal or non-treated mice (FIG. 12C). We also assessed expression of proinflammatory cytokines by quantitative RT-PCR analysis. As shown in FIG. 12, Aged mice at 72h post-LPS exhibit persistent increase of expression of TNF-α and IL-6 (D and E), indicating inflammation whereas the expression of these genes was markedly reduced in lungs of DMOG-treated mice. Thus, DMOG treatment can activate the vascular repair program and induce FoxM1 expression seen in young adult mice leading to resolution of inflammation in aged mice.

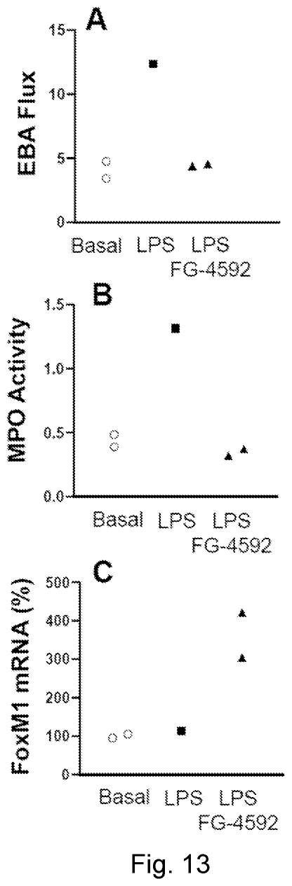

FG-4592 (i.e., roxadustat) is a DMOG analogue with more specific inhibition of prolyl hydroxylase 2. Roxadustat was recently tested for treatment of anemia in patients with chronic kidney disease (1). We also tested whether FG-4592 treatment could also activate vascular repair in aged mice. Aged mice (21 mo. old) were challenged with LPS and then treated with FG-4592 at 24h post-LPS and lung tissues were collected at 72h post-LPS. As shown in FIG. 13, FG-4592 treatment normalized vascular repair, inhibited MPO activity, and induced FoxM1 expression in aged mice in a manner similar to DMOG treatment. Given the effective activation of the vascular repair program in aged mice, DMOG analogues including roxadustat, molidustat, vadadustat, and desidustat can be used as a monotherapy or combination therapy with one or more of Dexamethasone, RV, NAC, NOX2 inhibitors (Apocynin, Ebselen, APX-115, Thienopyridine, NOX2ds-tat, NOX2 inhibiting nuclei acid), or decitabine to treat COVID-19, COVID-19 sepsis, COVID-19 respiratory distress and multi-organ failure, sepsis, and ARDS in adult patients and particularly in elderly patients to reduce morbidity and mortality and also for treatment of vascular diseases associated with impaired HIF-1a signaling and/or diminished FOXM1 expression including but not limited to restenosis and critical limb ischemia.

### REFERENCE FOR EXAMPLE 4

Joharapurkar A A, Pandya V B, Patel V J, Desai R C, Jain M R. Prolyl Hydroxylase Inhibitors: A Breakthrough in the Therapy of Anemia Associated with Chronic Diseases. J Med Chem. 2018 Aug. 23; 61(16):6964-6982.

Example 5: SIRT1 inhibitors (e.g., Selisistat, AG-1031, SIRT1 inhibiting nucleic acid) as a monotherapy or combination therapy with one or more of Dexamethasone, NAC, NOX2 inhibitors (Apocynin, Ebselen, APX-115, Thienopyridine, NOX2ds-tat, NOX2 inhibiting nucleic acid) to treat COVID-19, COVID-19 sepsis, COVID-19 respiratory distress and multi-organ failure, sepsis, ARDS, and multi-organ failure in elderly patients as well as for treatment of vascular diseases with hyperactivated SIRT1 signaling and/or diminished FOXM1 expression including but not limited to restenosis and critical limb ischemia.

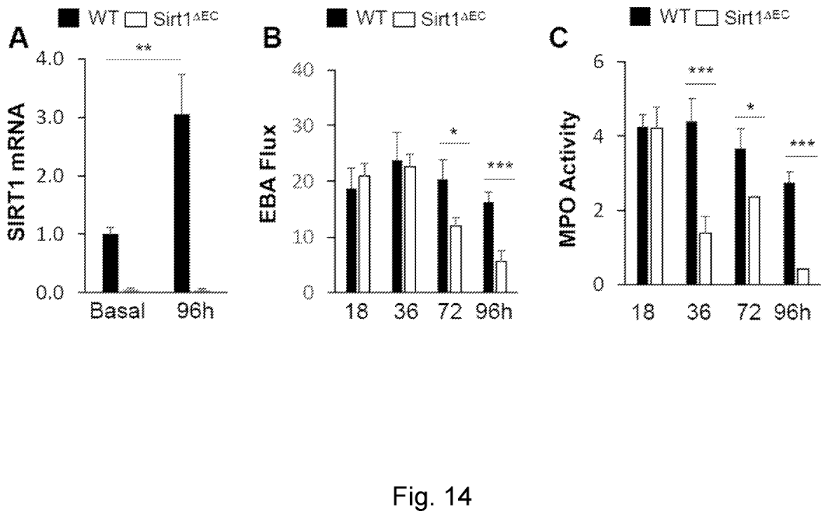

SIRT1 belongs to NAD⁺-dependent histone deacetylases (also called sirtuins, SIRT1-7) (1). Via deacetylation of both epigenetic and non-epigenetic targets, SIRT1 regulates the cell cycle, apoptosis, and oxidative stress response, thereby influences cell viability and aging (2). Published study shows that SIRT1 deficiency enhances lung inflammation following sepsis challenge in adult mice (3). To study the role of SIRT1 in regulating FoxM1 expression and vascular repair in aged mice, we first generated a mouse model with EC-restricted disruption of Sirt1 (Sirt1^{ΔEC}) (FIG. 14A). To our surprise, SIRT1 was markedly induced in lungs of aged WT mice at 96h post-LPS. As SIRT1 expression was inhibited in lungs of aged Sirt1^{ΔEC} mice at basal and 96h post-LPS, suggesting the induced SIRT1 expression in WT lungs is predominantly in lung ECs. WT and Sirt1^{ΔEC} mice at age of 20-24 months were challenged with LPS and lung tissues were collected at various times for assessment of lung vascular permeability and inflammation. As shown in FIG. 14B, vascular injury was peaked at 18 and 36h post-LPS in WT and Sirt1^{ΔEC} mice. After 36h, lung vascular permeability was decreased and returned to basal levels at 96h post-LPS in Sirt1^{ΔEC} mice. In contrast, WT mice exhibited persistent lung vascular injury, indicative of impaired recovery. Lung MPO activity was also fully recovered in Sirt1^{ΔEC} mice at 96h post-LPS but not in WT mice (FIG. 14C). Both WT and Sirt1^{ΔEC} mice exhibited similar degree of vascular injury and lung inflammation in the injury phase. The critical difference is that aged Sirt1^{ΔEC} mice exhibited normal vascular repair and resolution of inflammation as seen in young adult WT mice whereas aged WT mice exhibited defective vascular repair.

Our findings are novel and fundamentally important for drug development. We for the first time show that SIRT1 deficiency promote vascular repair and resolution of lung inflammation in aged mice which is in contrast with the literature that SIRT1 deficiency in young adult mice enhances inflammatory lung injury. Thus, SIRT1 has different (maybe opposite) functions at different ages in response to sepsis challenge. SIRT1 is an important regulator of the inflammatory response in young adult mice while it is a key inhibitor of vascular repair in aged mice. Thus, targeting SIRT1 may be a novel and important strategy to activate the dormant vascular repair process in aged subject to promote vascular repair and resolution of inflammation and thus promote survival.

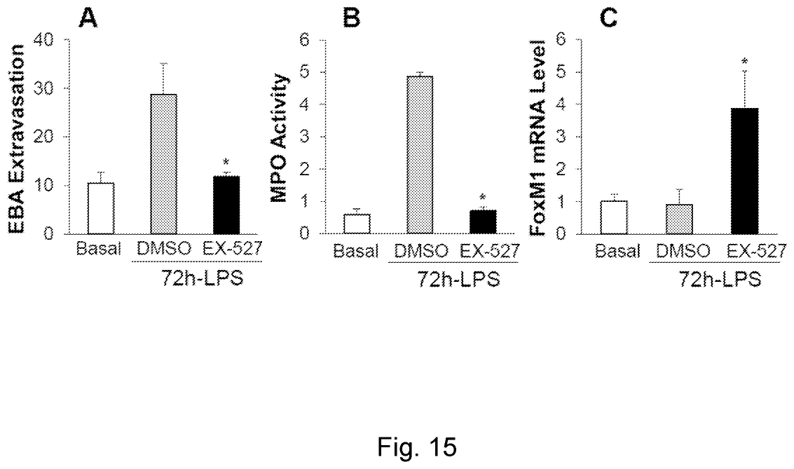

Next we addressed the possibility of pharmacological inhibition of SIRT1 to activate the intrinsic vascular repair program in aged mice. WT mice at age of 20 months were challenged with LPS and received treatment of EX-527 (i.e., Selisistat) or vehicle. At 72h post-LPS, lung tissues were collected for analysis. Vascular permeability in aged WT mice treated with EX-527 was at basal level in contrast to control WT mice (FIG. 15A). Similarly, lung MPO activity in EX-527-treated WT mice was also returned to basal levels whereas it remained elevated in vehicle-treated WT mice (FIG. 15B). EX-527 treatment reactivated FoxM1 expression aged lungs (FIG. 15C). Thus, inhibition of SIRT1 in aged subjects by either pharmacological approach (e.g., Selisistat, AG-1031 or their analogues) or SIRT1 inhibiting nucleic acid including, but not limited to antisense oligo, siRNA, shRNA, or guide RNA can reactivate the intrinsic vascular repair program seen in young adult subjects. SIRT1 inhibitors (e.g., Selisistat, AG-1031 or their analogues) or SIRT1 inhibiting nucleic acid as a monotherapy or combination therapy with one or more of Dexamethasone, NAC, NOX2 inhibitors (Apocynin, Ebselen, APX-115, Thienopyridine, NOX2ds-tat, NOX2 inhibiting nucleic acid) to treat COVID-19, COVID-19 sepsis, COVID-19 respiratory distress and multi-organ failure, sepsis, ARDS, and multi-organ failure in elderly patients and also vascular diseases including restenosis and critical limb ischemia.

### REFERENCE FOR EXAMPLE 5

Jing H, Lin H. Sirtuins in epigenetic regulation. Chem Rev. 2015. 115: 2350-75.
Grabowska W, Sikora E, Bielak-Zmijewska A. Sirtuins, a promising target in slowing down the ageing process. Biogerontology. 2017, 18: 447-476.
Gao R, Ma Z, Hu Y, Chen J, Shetty S, Fu J. Sirt1 restrains lung inflammasome activation in a murine model of sepsis. Am J Physiol Lung Cell Mol Physiol. 2015, 308: L847-53.

Example 6. Aging exaggerates inflammatory lung injury.

It has been shown that the incidence of acute lung injury (ALI)/ARDS resulting from sepsis is as much as 20-fold greater in elderly patients (≥60 yr) than in young adult patients, and the mortality rate of elderly ALI/ARDS patients is also up to 20-fold greater (1-6). The severity and mortality of COVID-19 in elderly patients are 10-100 fold greater (7, 8). Per CDC report, the overall cumulative hospitalization rate of COVID-19 patients in US between Mar. 1, 2020 and May 8, 2020 is 503 per million, with the highest rates in people 65 years and older (1622 per million) and 50-64 years (790 per million). 8 out of 10 deaths from SARS-CoV2 infection reported in the U.S have been in adults 65 years old and older. In Italy, the death rates of COVID-19 patients by May 6, 2020 are 0.1%-0.9%, 2.5%, 10.1%, and 25% or more in age group of 20-49, 50-59, 60-69, and ≥70 years old, respectively. However, the underlying causes of aging effects are poorly understood and current therapy is supportive. Here, our present invention provides a treatment that could markedly inhibit lung injury and inflammation and promote survival. Furthermore, combination therapy with the injury inhibitors and reparative activators is likely an effective therapeutic approach for COVID-19, COVID-19 sepsis, COVID-19 respiratory distress and multi-organ failure, sepsis, and ARDS in elderly patients. Coupled with anti-viral therapy, this novel cocktail therapy may hold great promise for effective treatment of COVID-19 and promote survival.

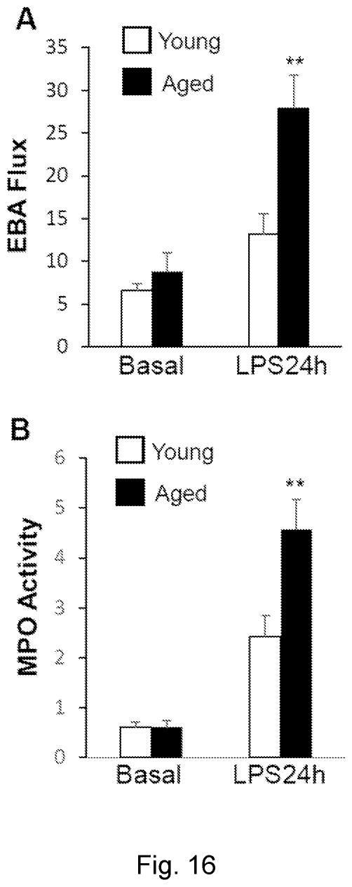

To determine the injury response of young adult and aged mice, we challenged adult (3 mo. old) and aged (19 mo. old) mice with the same dose of lipopolysaccharide (LPS) (5 mg/kg, i.p., LPS from Sigma Aldrich). At 24h post-LPS, lung tissues were collected for EBA and MPO activity assays. As shown in FIG. 16A, lung EBA flux, a measurement of vascular permeability to protein was similar between young adult and aged mice at basal. At 24h post-LPS, EBA was markedly increased in both young adult mice and aged mice. However, EBA increase was much greater in aged mice than young adult mice, demonstrating severe lung vascular injury. MPO activity was also similar at basal condition and markedly increased in response to LPS challenge (FIG. 16B). Again, lung MPO activity was drastically greater in aged mice compared to young adult mice at 24h post-LPS. Together these data provide unequivocal evidence that aged mice are more susceptible to sepsis and exhibit severe inflammatory lung injury.

### REFERENCE FOR EXAMPLE 6

1. Rubenfeld G D, et al. Incidence and outcomes of acute lung injury. The New England journal of medicine 353, 1685-1693 (2005).
2. Angus D C, Linde-Zwirble W T, Lidicker J, Clermont G, Carcillo J, Pinsky M R. Epidemiology of severe sepsis in the United States: analysis of incidence, outcome, and associated costs of care. Critical care medicine 29, 1303-1310 (2001).
3. M. R. Suchyta et al., Increased mortality of older patients with acute respiratory distress syndrome. Chest 111, 1334-1339 (1997).
4. Gee M H, Gottlieb J E, Albertine K H, Kubis J M, Peters S P, Fish J E. Physiology of aging related to outcome in the adult respiratory distress syndrome. Journal of Applied Physiology 69, 822-829 (1990).
5. E. W. Ely et al., Recovery rate and prognosis in older persons who develop acute lung injury and the acute respiratory distress syndrome. Annals of Internal Medicine 136, 25-36 (2002).
6. Griffith D, Idell S. Approach to adult respiratory distress syndrome and respiratory failure in elderly patients. Clinics in chest medicine 14, 571-582 (1993).
7. The Novel Coronavirus Pneumonia Emergency Response Epidemiology Team, Chinese Center for Disease Control and Prevention. The epidemiological characteristics of an outbreak of 2019 novel coronavirus disease (COVID-19) in China. Chin J Epidemiol. 41, 145-151 (2020).
8. CDC Response Team. Severe outcomes among patients with coronavirus disease 2019 (COVID-19): United States Mar. 1 to May 8, 2020. https://www.cdc.gov/coronavirus/2019-ncov/covid-data/covidview/index.html.

Example 7. NOX2 is markedly increased in aged lung ECs and inhibition of NOX2 markedly inhibits inflammatory lung injury in aged mice. Thus, NOX2 inhibitors including but limited to Thienopyridine, NOX2 inhibiting peptide or nucleic acids, or pan-NOX inhibitors Apocynin, Ebselen, or APX-115 as a monotherapy or combination therapy with either Selisistat, AG-1031, and/or rabeprazol, and/or phenazopyridine, and/or DMOG analogues roxadustat or molidustat, or vadadustat, or desidustat, and/or decitabine as well as SIRT1 inhibiting nucleic acid, EGLN1 inhibiting nucleic acid, HIF-1a expressing nucleic acid, or FOXM1 expressing nucleic acid are useful to treat COVID-19, COVID-19 sepsis, COVID-19 respiratory distress and multi-organ failure, sepsis, ARDS, and multi-organ failure in elderly patients as well as vascular diseases associated with NOX2 hyperactivity.

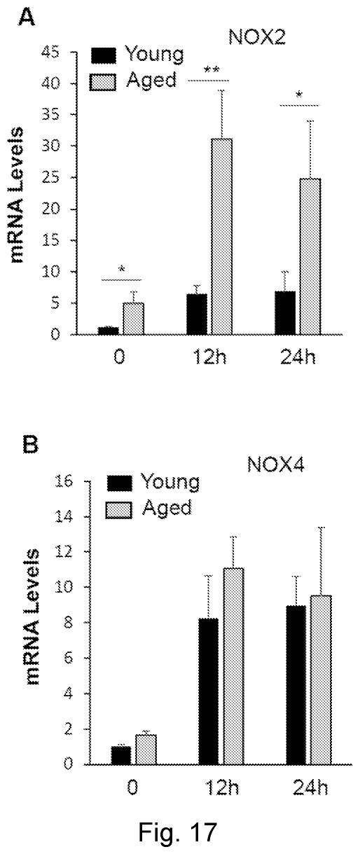

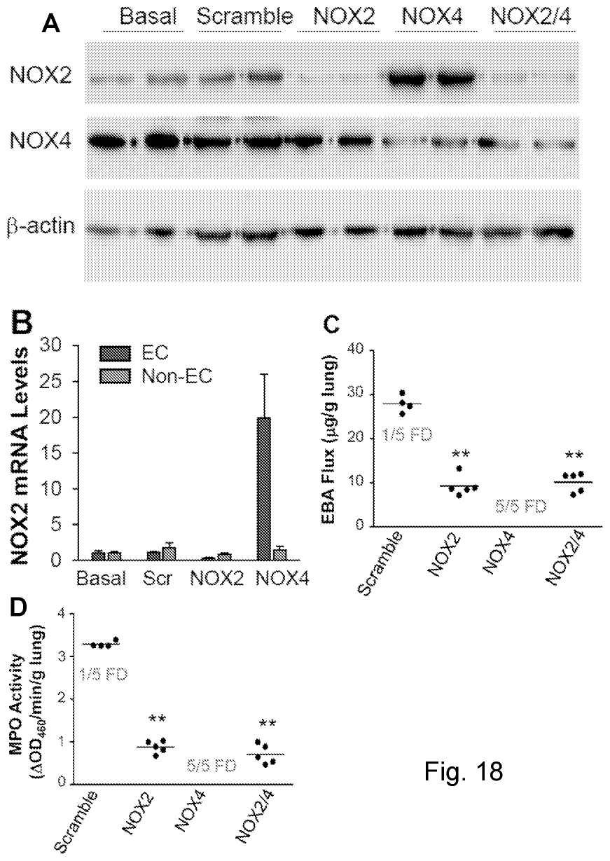

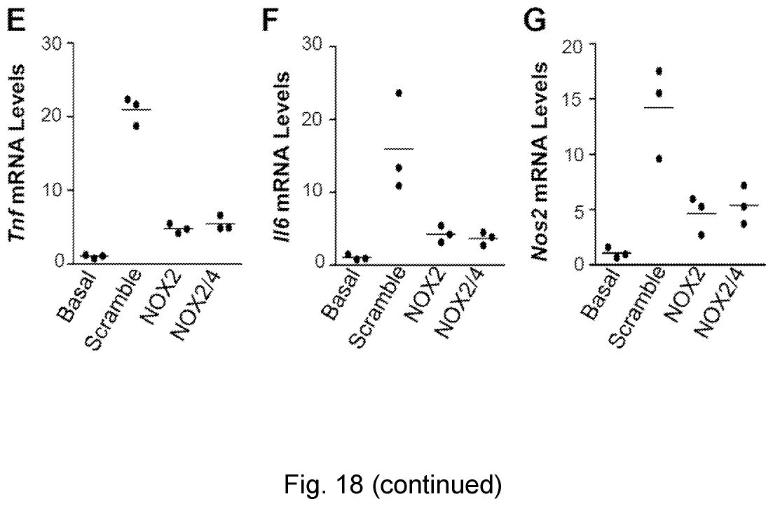

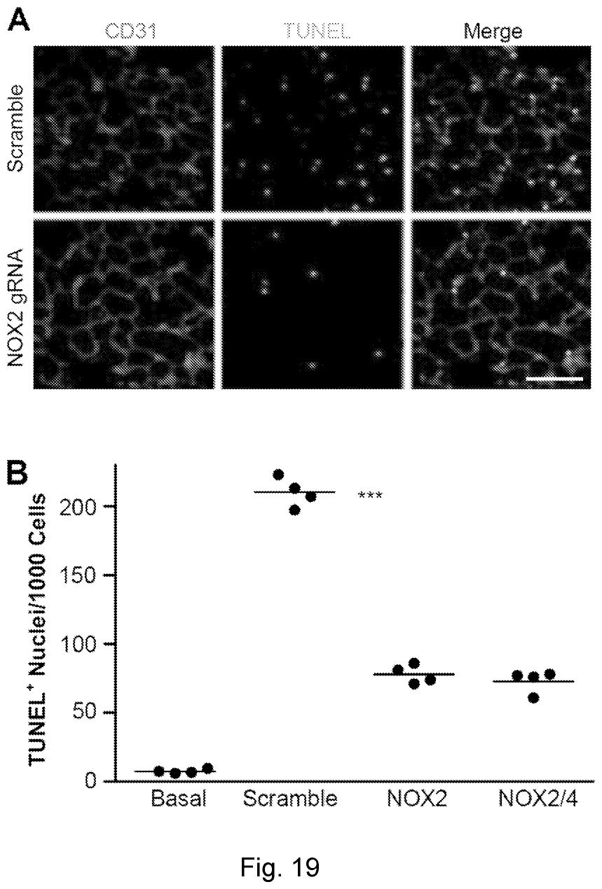

NADPH oxidase (NOX) family of enzymes including NOX1-5 and dual oxidase DUOX1 and 2 catalyze the reduction of 02 to reactive oxygen species (ROS), and excessive ROS have been associated with tissue damage (1, 2). NOX2 also known as gp91phox was first discovered in phagocytes, and serves as an important inflammatory mediator against invading bacteria (3). NOX 4 which most generates H₂O₂ is highly expressed in fibroblasts and vascular smooth muscle cells and play an important role in vascular remodeling and pulmonary fibrosis (4). Inhibition of NOX4 is under clinical trial for human idiopathic pulmonary fibrosis. We have studied the expression changes of NOX2 and NOX in lungs of aged mice at basal and following sepsis challenge. As shown in FIG. 17. NOX2 but not NOX4 expression was markedly induced in the lungs of aged mice at basal compared to young adult mice. In response to LPS challenge, NOX2 expression was induced in the lungs of both young adult and aged mice. But, the induction was much greater in aged lungs. To gain insights into the role of NOX2 and NOX4 in regulating the severity of lung injury in aged mice, we employed a CRISPR/Cas9-mediated genome editing approach to knockdown their expression in aged mice. 7 days after i.v. delivery of mixture of PLGA-PEG/PEI nanoparticles:plasmid DNA expressing Cas9 under the control of CDH5 promoter (EC-specific) and guide RNA specific for either NOX2 or NOX4 driven by U6 promoter, lung tissues were collected for gene expression analysis. NOX2 and NOX4 protein expression was efficiently knocked down by NOX2 and NOX4 guide RNA, respectively (FIG. 18A). Interestingly, knockdown of endothelial NOX4 resulted in a marked induction of NOX2 at both protein and mRNA levels (FIG. 18, A and B) selectively in ECs. Then the mice were challenged with LPS. At 24h post-LPS challenge, aged WT mice (treated with scramble guide RNA) exhibited severe vascular injury with an EBA value of 28 and 1 of 5 mice died. Surprisingly, all aged mice with knockdown of endothelial NOX2 survived and the EBA value was around 10 which was very close to basal levels (normally 5-7), indicating minor vascular injury in NOX2 EC-deficient mice. However, NOX4 knockdown resulted in 100% mortality following LPS challenge. The aged mice with knockdown of both NOX2 and NOX4 exhibited a phenotype similar to NOX-2 deficient mice (FIG. 18C). Similarly, aged WT mice with scrambled guide RNA treatment exhibited severe lung inflammation evident by markedly increased MPO activity and expression of proinflammatory cytokines whereas NOX2 or NOX2 and NOX4 knockdown largely inhibited lung inflammation in response to the same dose of LPS challenge (FIG. 18, D-G). Consistently, EC apoptosis seen in WT mice was also markedly inhibited in NOX-2 or NOX2/4-deficient mice at 24h post-LPS (FIG. 19).

Together, these data for the first time demonstrate that aging-dependent increase of NOX2 expression in lung ECs are responsible for the augmented inflammatory lung injury in aged mice in response to LPS challenge whereas NOX4 is protective. Marked increase of NOX2 induced by NOX4 deficiency accounts for the great mortality in NOX4-deficient mice. Thus, inhibition of NOX2 in aged subjects by NOX2 inhibitors including but not limited Thienopyridine, NOX2 inhibiting peptide (e.g., NOX2ds-tat) and NOX2 inhibiting nucleic acid including antisense, siRNA, shRNA and guide RNA, and pan-NOX inhibitor Apocynin, Ebselen, APX-115 as a monotherapy to inhibit inflammatory lung injury and as a combination therapy with one or more of Selisistat, AG-1031, and/or rabeprazol, and/or phenazopyridine, and/or DMOG analogues roxadustat or molidustat, or vadadustat, or desidustat, and/or decitabine or SIRT1 inhibiting nucleic acid, EGLN1 inhibiting nucleic acid, FOXM1 expressing nucleic acid, or HIF-1a expressing nucleic acid to promote vascular repair and thus effectively treat COVID-19, COVID-19 sepsis, COVID-19 respiratory distress and multi-organ failure, sepsis, ARDS, and multi-organ failure in elderly patients. Selective inhibition of NOX4 will worsen the disease and increase mortality.

### REFERENCES FOR EXAMPLE 7

1. Lambeth, J. D. NOX enzymes and the biology of reactive oxygen. Nat. Rev. Immunol. 2004, 4: 181-189.
2. Bedard, K. & Krause, K. H. The NOX family of ROS-generating NADPH oxidases: physiology and pathophysiology. Physiol. Rev. 2007, 87: 245-313.
3. Geiszt, M. & Leto, T. L. The Nox family of NAD(P)H oxidases: host defense and beyond. J. Biol. Chem. 2004, 279: 51715-51718.
4. Hecker L, Vittal R, Jones T, Jagirdar R, Luckhardt T R, Horowitz J C, Pennathur S, Martinez F J, Thannickal V J. NADPH oxidase-4 mediates myofibroblast activation and fibrogenic responses to lung injury. Nat Med. 2009, 15:1077-1081.

Example 8. N-Acetylcysteine (NAC) as a monotherapy or combination therapy with one or more of Selisistat, AG-1031 and their analogues, and/or rabeprazol, and/or phenazopyridine, and/or DMOG analogues roxadustat or molidustat, or vadadustat, or desidustat, and/or decitabine (e.g. Dacogen, INDOVI) or azacytidine (e.g., Vidiaz, ONUREG), or SIRT1 inhibiting nucleic acid, EGLN1 inhibiting nucleic acid, FOXM1 expressing nucleic acid, or HIF-1a expressing nucleic acid to treat COVID-19, COVID-19 sepsis, COVID-19 respiratory distress and multi-organ failure, sepsis, ARDS, and multi-organ failure in elderly patients.

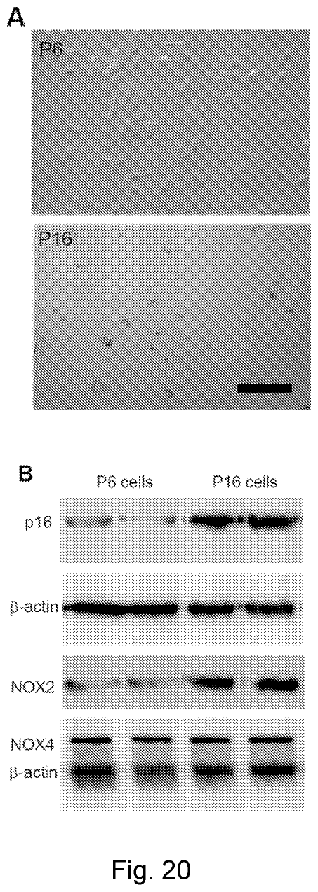

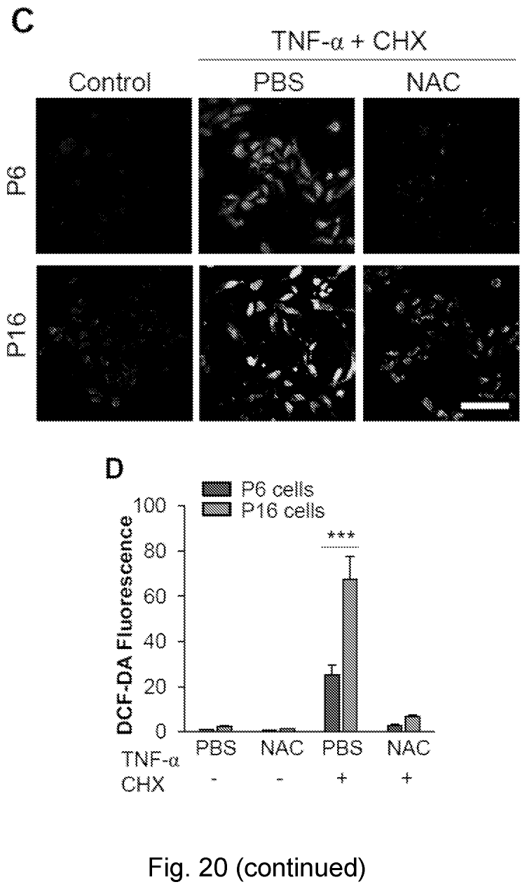

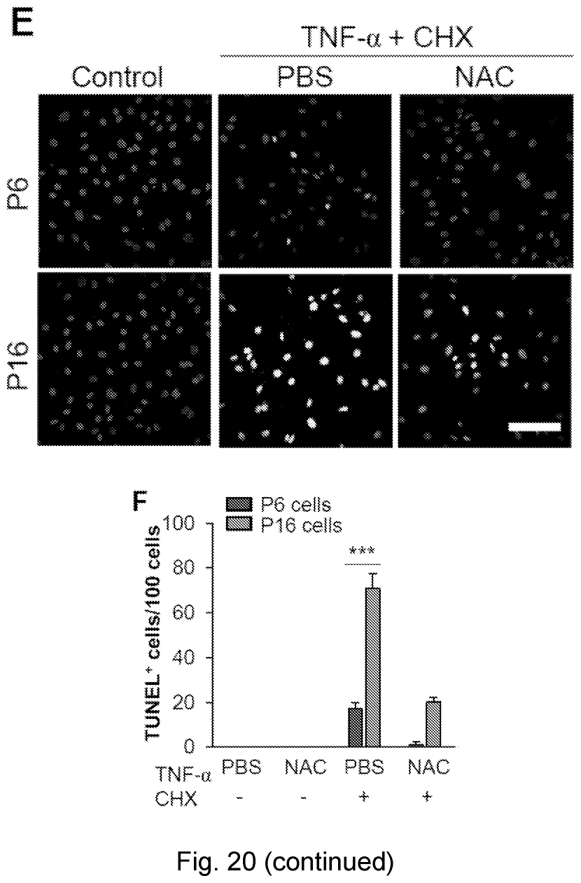

To further understand the pathogenic role of aging-induced NOX2 in promoting inflammatory lung injury in aged mice, we cultured human lung microvascular ECs (HLMVECs) and passaged many times. We found that HLMVECs at passage 16 became senescent evident by prominent β-galactosidase staining (FIG. 20A), which is a well-known marker of cell senescence. Expression of NOX2 but not NOX4 was markedly increased in these senescent ECs compared to normal ECs at passage 6 (FIG. 20B), which is consistent with our observation in lungs of aged WT mice (FIG. 17A). Consistent with the function of NOX2 as a ROS generator, senescent HLMVECs at passage 16 generated much more ROS in response to TNF-α and Cycloheximide (CHX) treatment, which was inhibited by NAC treatment (FIG. 20C). Accordingly, TNF-α/CHX treatment-induced cell apoptosis was also markedly inhibited by NAC treatment in senescent cells (FIG. 20D). These data demonstrate that NAC is an effective anti-oxidant in aged lungs to inhibit aging-dependent severe inflammatory lung injury induced by sepsis.

NAC treatment normalizes inflammatory lung injury in aged mice to the levels similar to young adult mice.

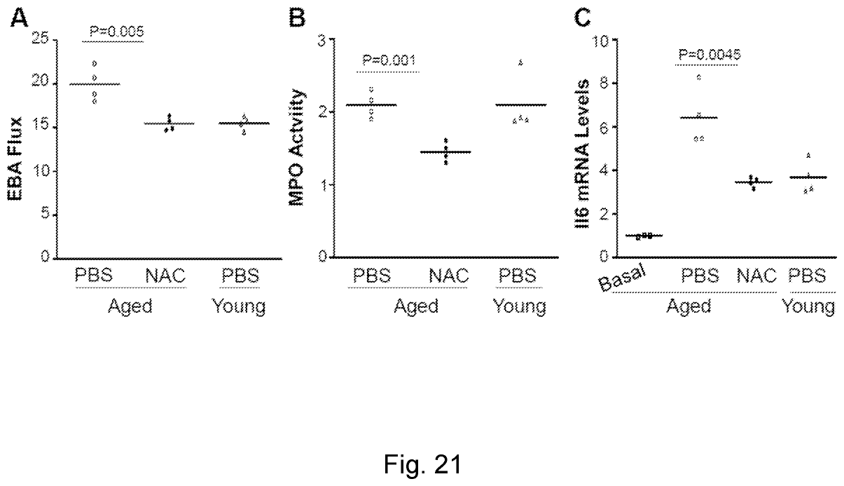

To determine whether NAC treatment in aged mice can attenuate lung injury in aged mice, aged mice (21.5 mos. old) and young adult WT mice (3 mos. old) were challenged with LPS (2 mg/kg, i.p.) and then treated with NAC (120 mg/kg, oral) or PBA at 2 h post-LPS. Lung tissues were collected at 24h post-LPS for analyses. As shown in FIG. 21A, NAC treatment attenuated EBA flux (i.e. vascular permeability) in aged mice to a level similar to young adult mice. It also attenuated MPO activity in aged lungs (FIG. 21B). Expression of the proinflammatory gene 116 in lungs of NAC-treated aged mice was also reduced to a level similar to young adult mice (FIG. 21C).

Thus, NAC can be used as a monotherapy or more importantly, combination therapy with one or more of Selisistat, AG-1031 and their analogues, and/or rabeprazol, and/or phenazopyridine, and/or DMOG analogues roxadustat or molidustat, or vadadustat, or desidustat, and/or decitabine (e.g., Dacogen, INQOVI) or azacitidine (e.g., Vidiaz, ONUREG), or SIRT1 inhibiting nucleic acid, EGLN1 inhibiting nucleic acid, FOXM1 expressing nucleic acid, or HIF-1a expressing nucleic acid to treat COVID-19, COVID-19 sepsis, COVID-19 respiratory distress and multi-organ failure, sepsis, ARDS, and multi-organ failure in elderly patients.

Example 9. Resveratrol as a monotherapy or combination therapy with one or more of rabeprazole, or phenazopyridine, or roxadustat or molidustat, or vadadustat, or desidustat, or decitabine, or EGLN1 inhibiting nucleic acid, FOXM1 expressing nucleic acid, or HIF-1a expressing nucleic acid to treat COVID-19, COVID-19 sepsis, COVID-19 respiratory distress and multi-organ failure, sepsis, ARDS, and multi-organ failure in patients.

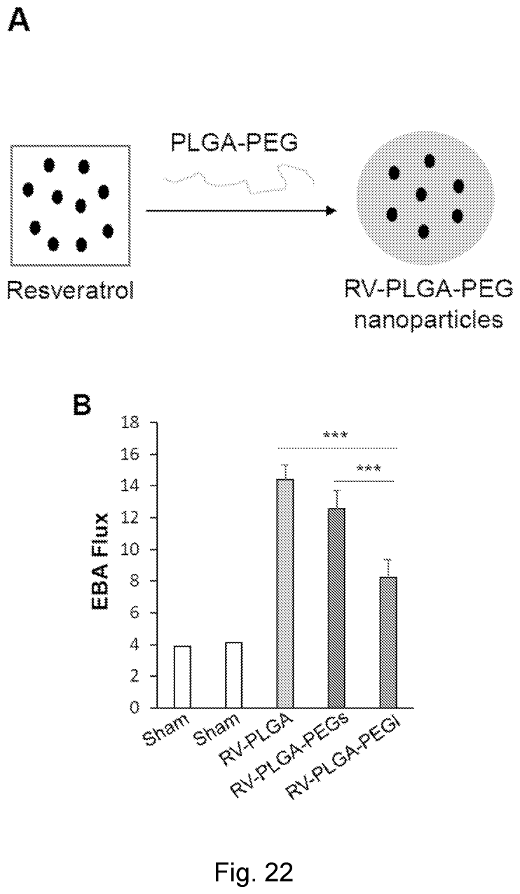

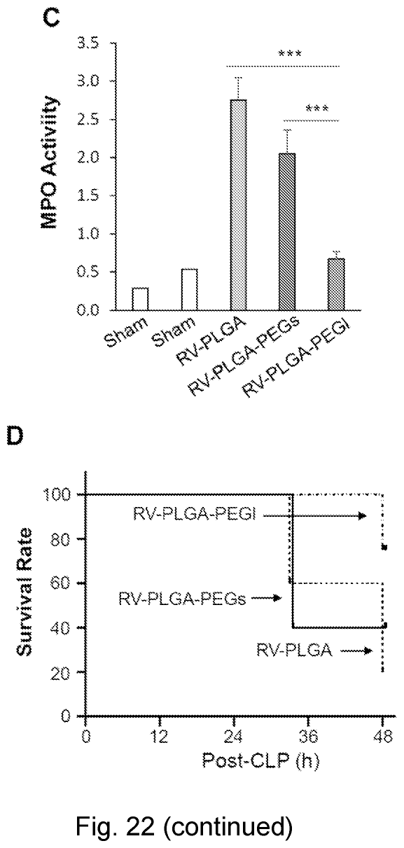

Resveratrol has been reported to have anti-inflammatory, anti-oxidant and anti-cancer properties. However, its use is widely hindered by its poor solubility. We formulated 3 RV-loaded nanoparticles comprised of PLGA (MW=25,000 Da), and PLGA-PEG600 (PEGs), or PLGA-PEG2000 (PEGl) (FIG. 22A). Adult mice (3-5 months old) were subjected to either sham operation (sham) or cecal ligation and puncture (CLP) to induce polymicrobial sepsis. At 3h post-CLP, the mice were treated with RV-PLGA, RV-PLGA-PEGs, or RV-PLGA-PEGl (5 mg/kg of RV, i.v.). At 36h post-CLP, lung tissues were collected for assessment of lung vascular permeability (EBA Flux) and inflammation (MPO activity). As shown in FIG. 22, B and C, RV-PLGA-PEG1 was the most efficient formulation in inhibiting inflammatory lung injury. Accordingly, 3 of 4 mice treated with RV-PLGA-PEGl nanoparticles survived at 48h post-CLP whereas only 1 of 5 mice treated with RV-PLGA nanoparticles and 2 of 5 mice treated with RV-PLGA-PEGs nanoparticles survived. Thus, the present invention identifies specific formulation of RV-loaded nanoparticles as a monotherapy or combination therapy with one or more of rabeprazol, or phenazopyridine, or roxadustat or molidustat, or vadadustat, or desidustat, or decitabine, or Selisistat, AG-1031, or SIRT1 inhibiting nucleic acid, EGLN1 inhibiting nucleic acid, FOXM1 expressing nucleic acid, or HIF-1a expressing nucleic acid to treat COVID-19, COVID-19 sepsis, COVID-19 respiratory distress and multi-organ failure, sepsis, ARDS, and multi-organ failure in patients.

Example 10. Decitabine and its analogues (e.g., Dacogen, INQOVI, Vidaza, NUREG) as a monotherapy or combination therapy with one or more of Resveratrol, NAC, NOX2 inhibitor (e.g. Thienopyridine, NOX2 inhibiting peptide (e.g., NOX2ds-tat) and pan-NOX inhibitor Apocynin, Ebselen, APX-115), Selisistat, AG-1031, rabeprazole, phenazopyridine, or DMOG analogues roxadustat or molidustat, or vadadustat, or desidustat, or NOX2 inhibiting nucleic acid, SIRT1 inhibiting nucleic acid, EGLN1 inhibiting nucleic acid, or HIF-1a expressing nucleic acid for the treatment of COVID-19, COVID-19 sepsis, COVID-19 respiratory distress and multi-organ failure, sepsis, ARDS, and multiple organ failure in elderly patients as well as vascular diseases associated with diminished FOXM1 expression including but not limited to restenosis and critical limb ischemia.

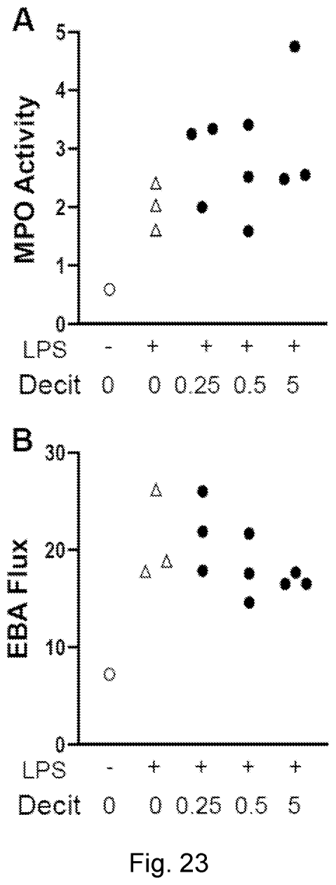

Decitabine has no effect on sepsis-induced lung injury in young adult mice. Decitabine is used for treatment of patients with myelodysplastic syndrome (MDS). Previously study has shown that 5′-Aza-2′-deoxycytidine (Aza, Decitabine) (1 mg/kg, i.p.) has no effects on sepsis-induced inflammatory lung injury except reduced lung injury by combined use of both Aza and Trichostatin A (TSA, a histone deacetylase inhibitor) in young adult mice (8-10 weeks old) (1). Our study also shows that decitabine at various doses (0.25, 0.5, and 5 mg/kg, i.p.) has no effect on sepsis-induced inflammatory lung injury in young adult mice (3-5 mo. old) evident by lung MPO activity (FIG. 23A) and lung EBA flux (FIG. 23B).

Decitabine reactivation of FoxM1-dependent endothelial regeneration and vascular repair in lungs of aged mice but not of young adult mice.

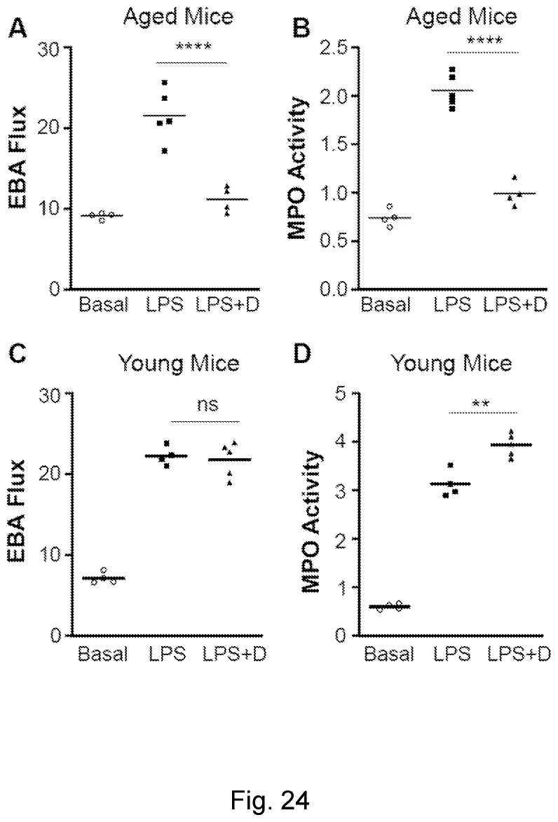

We next explored the possibility of Decitabine reactivation of FoxM1-dependent endothelial regeneration in aged lungs which will have great translational potential for treatment of ARDS and severe COVID-19 in elderly patients. At 24 h and 48h post-LPS challenge, the aged (21-22 mos. old) mice were treated with Decitabine (0.2 mg/kg, i.p.) or vehicle (PBS) and lung tissues were collected at 96h post-LPS for analyses. EBA assay demonstrated normalized vascular repair in Decitabine-treated aged mice in contrast to vehicle-treated aged mice (FIG. 24A). Lung MPO activity of Decitabine-treated aged mice was also returned to basal levels whereas vehicle-treated mice exhibited markedly elevated MPO activity (FIG. 24B). However, Decitabine treatment didn't promote vascular repair and inflammation resolution in young adult mice (FIG. 24, C and D).

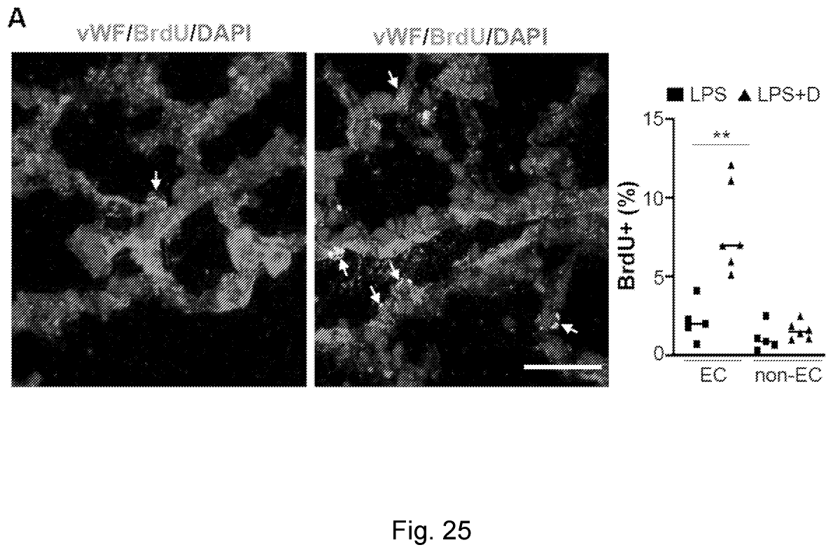

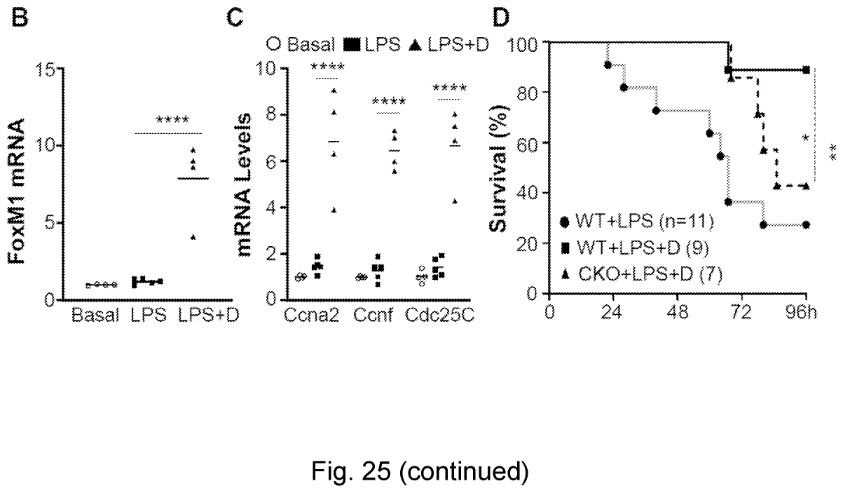

BrdU immunostaining revealed that pulmonary vascular EC proliferation was drastically increased in Decitabine-treated aged mice, indicating reactivation of endothelial regeneration in aged lungs (FIG. 25A).

Quantitative RT-PCR analysis shows FoxM1 expression was markedly induced in lungs of Decitabine-treated mice at 72h post-LPS compared to vehicle-treated mice (FIG. 25B). Accordingly, expression of FoxM1 target genes essential for cell cycle progression were also markedly induced in lungs of Decitabine-treated aged mice (FIG. 25C). Decitabine treatment also markedly improve survival of aged WT mice. 80% of Decitabine-treated mice survived whereas only 20% of vehicle-treated WT mice survived at the same period (FIG. 26D). To determine if the survival effect was mediated by endothelial expression of FoxM1, we employed the mice with EC-specific knockout out of Foxm1 (Foxm1 KO). As shown in FIG. 26D, Decitabine treatment had no protective effects on the survival of aged Foxm1 KO mice following LPS challenge.

Together, these data suggest that decitabine and its analogues can be repurposed to reactivate endothelial regeneration and vascular repair and promote recovery and thereby reduce morbidity and mortality of elderly patients with either COVID-19 and COVID-19 respiratory distress, sepsis, and/or multi-organ failure, sepsis, ARDS, or multiple organ failure as a monotherapy or combination therapy with one or more of Resveratrol, NAC, NOX2 inhibitors (e.g. Thienopyridine, NOX2 inhibiting peptide (e.g., NOX2ds-tat), pan-NOX inhibitors (e.g., Apocynin, Ebselen, APX-115), Selisistat, AG-1031, rabeprazol, phenazopyridine, or DMOG analogues roxadustat or molidustat, or vadadustat, or desidustat, NOX2 inhibiting nucleic acid, EGLN1 inhibiting nucleic acid, SIRT1 inhibiting nucleic acid, HIF-1a expressing nucleic acid, or FOXM1 expressing nucleic acid.

### REFERENCES FOR EXAMPLE 10

1. Thangavel J, Malik A B, Elias H K, Rajasingh S, Simpson A D, Sundivakkam P K, Vogel S M, Xuan Y T, Dawn B, Rajasingh J. Combinatorial therapy with acetylation and methylation modifiers attenuates lung vascular hyperpermeability in endotoxemia-induced mouse inflammatory lung injury. Am J Pathol. 2014, 84: 2237-49.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method of treating
(i) one or more of COVID-19, COVID-19-related sepsis, COVID-19-related respiratory distress and organ failure in a subject in need thereof, or
(ii) one or more of sepsis, acute respiratory distress syndrome (ARDS), acute inflammatory injury, and infection-induced organ failure characterized by increased lung microvascular permeability in a subject in need thereof, or
(iii) one or more of vascular diseases associated with impaired endothelial regeneration, vascular repair, and vascular regeneration including restenosis following percutaneous coronary intervention, peripheral vascular diseases including critical limb ischemia, in a subject in need thereof,
the method comprising: administering to the subject an effective amount of (a) a compound that inhibits endothelial injury and inflammation; and/or (b) a compound that promotes endothelial regeneration, vascular repair, and optionally resolution of inflammation.

2-3. (canceled)

4. The method of claim 1(iii), wherein the compound that promotes endothelial regeneration and vascular repair comprises one or more compounds selected from the group consisting of: rabeprazole and its analogues, Phenazopyridine and its analogues, Selisistat and its analogues, AG-1031 and its analogues, decitabine (e.g. Dacogen, INQOVI) and its analogues (e.g., Vidaza, ONUREG), SIRT1 inhibiting nucleic acid, EGLN1 inhibiting nucleic acid, HIF1A expressing nucleic acid, and FOXM1 expressing nucleic acid.

5. The method of claim 1(iii), wherein the compound that promotes endothelial regeneration and vascular repair comprises one or more compounds selected from the group consisting of roxadustat, molidustat, vadadustat, desidustat.

6. A method of treating anemia in a subject in need thereof, the method comprising: administering to the subject an effective amount of (a) rabeprazole or analogs thereof; and/or (b) Phenazopyridine or analogs thereof, or (c) a combination of one of rabeprazole and its analogues with one of Phenazopyridine and its analogues.

7. The method of claim 1(i) or 1(ii), wherein the compound that inhibits endothelial injury and inflammation comprises one or more compounds selected from the group consisting of: Dexamethasone, N-acetyl cysteine (NAC), NOX2 inhibitors (e.g., Thienopyridine, NOX2ds-tat,), pan-NOX inhibitors (e.g., Apocynin, Ebselen, APX-115), Reseveratrol (trans-E-resveratrol) nanoparticles and analogues thereof (e.g., Reseveratrol-loaded nanoparticles comprising of poly(D,L-lactic-co-glycolic acid) (PLGA)-b-long linker poly(ethylene glycol) (e.g., PEG_{MW2000, 5000Da}) copolymer or poly(D,L-lactic acid) (PLA)-b-PEG (e.g., PEG_{2000, 5000Da}) copolymer, and NOX2 inhibiting nucleic acid.

8. The method of claim 1(i), wherein the compound that promotes endothelial regeneration and vascular repair comprises one or more compounds selected from the group consisting of: Decitabine (e.g. Dacogen, INQOVI) and its analogues (e.g., Vidaza, ONUREG), roxadustat, molidustat, vadadustat, desidustat, Sirtuinl inhibitors (e.g., Selisistat and its analogues, AG-1031 and its analogues) and Sirtuinl inhibiting nucleic acid, rabeprazole (e.g., Aciphex) and its analogues, phenazopyridine (e.g., Pyridium) and its analogues; EGLN1 inhibiting nucleic acid, HIF-1α expressing nucleic acid, and FoxM1 expressing nucleic acid.

9. The method of claim 1(ii), wherein the compound that promotes endothelial regeneration and vascular repair comprises one or more compounds selected from the group consisting of: Decitabine (e.g. Dacogen, INQOVI) and its analogues (e.g., Vidaza, ONUREG), roxadustat, molidustat, vadadustat, desidustat, Sirtuinl inhibitors Selisistat and its analogs, AG-1031 and its analogs, and Sirtuinl inhibiting nucleic acid, rabeprazole (e.g., Aciphex) and its analogues, phenazopyridine (e.g., Pyridium) and its analogues; EGLN1 inhibiting nucleic acid, and FoxM1 expressing nucleic acid.

10. The method of claim 1(ii), wherein the sepsis and/or ARDS is caused by one or more of inhalation of a harmful substance, burn injury, major trauma with shock, and infection including bacterial infection, viral infection.

11. The method of claim 1, wherein the method comprises administering one or more compounds selected from the group consisting of:
a) a FOXM1 expressing nucleic acid;
b) an HIF-1α expressing nucleic acid;
c) a SIRT1 inhibiting nucleic acid;
d) an EGLN1 inhibiting nucleic acid; and
e) a NOX-2 inhibiting nucleic acid.

12. The method of claim 11, wherein the inhibitory nucleic acid is one or more, selected from the group consisting of: an antisense oligonucleotide; a small interfering RNA (siRNA) oligonucleotide, a guide RNA oligonucleotide; DNA that expresses a short hairpin RNA (shRNA), a guide RNA, a genome editing system, a mutant sequence comprising the dominant negative gene.

13. The method of claim 1(i) wherein treatment comprises administering an effective amount of one or more of NOX2 inhibiting nucleic acid, SIRT1 inhibiting nucleic acid, EGLN1 inhibiting nucleic acid, HIF1A expressing nucleic acid, FOXM1 expressing nucleic acid, resveratrol, NAC, Apocynin, Ebselen, APX-115, NOX2 inhibiting peptide (NOX2ds-tat), Thienopyridine, Selisistat, AG-1031, rabeprazole, phenazopyridine, roxadustat, molidustat, vadadustat, desidustat, and Decitabine (Dacogen, INQOVI, Vidaza, ONUREG), and analogs thereof.

14. The method of claim 1(ii) wherein treatment comprises administering an effective amount of one or more of NOX2 inhibiting nucleic acid, SIRT1 inhibiting nucleic acid, EGLN1 inhibiting nucleic acid, HIF1A expressing nucleic acid, FOXM1 expressing nucleic acid, resveratrol, NAC, Apocynin, Ebselen, APX-115, NOX2 inhibiting peptide (NOX2ds-tat), Thienopyridine, Selisistat, AG-1031, rabeprazol, phenazopyridine, and Decitabine (Dacogen, INQOVI, Vidaza, ONUREG), and analogs thereof.

15-37. (canceled)

38. The method of claim 1, wherein the subject is human.

39. The method of claim 38, wherein the subject is any age.

40. The method of claim 1, wherein the subject is at least 60 years old.

41-60. (canceled)

61. A nanoparticle comprising resveratrol, and one or more of:
(i) (a) poly(D,L-lactic-co-glycolic acid)-b-poly(ethylene glycol) (PLGA-b-PEG) copolymer; and (b) poly(D,L-lactic acid)-b-poly(ethylene glycol) (PLA-b-PEG) copolymer; or
(ii) poly(D,L-lactic-co-glycolic acid) (PLGA) and poly(D,L-lactic acid) (PLA) polymer.

62. (canceled)

63. The nanoparticle of claim 61, wherein the nanoparticle is coated with poly(ethylene glycol).

64. The nanoparticle of claim 61, wherein the molecular weight of PLGA is from about 1000 to about 100,000 Da, or about 25,000 Da, about 55,000 Da; and wherein the molecular weight of PLA is from about 1000 to about 50,000 Da, about 10,000 Da, or about 20,000 Da.

65. The nanoparticle of claim 63, wherein the molecular weight of PEG is from about 1,000 to about 20,000 Da, or is about 2,000, about 3,000, about 4,000, about 5,000, about 6,000, about 7,000, or about 8,000 Da.

66-69. (canceled)
